SEQUENCE LISTING (36 sequences)

SEQ ID NO: 1 - DNA, 20 bp, Unknown
  Description of Unknown Nucleating polypeptide binding motif sequence
  modified_base (1)..(1): a, c, t or g
  modified_base (3)..(3): a, c, t or g
nbndccdshagrkgghrshv

SEQ ID NO: 2 - DNA, 20 bp, Unknown
  Description of Unknown Nucleating polypeptide binding motif sequence
  modified_base (18)..(18): a, c, t or g
  modified_base (20)..(20): a, c, t or g
vhsrhggkrgahsdccdnbn

SEQ ID NO: 3 - DNA, 7 bp, Artificial Sequence
  Description of Artificial Sequence Synthetic oligonucleotide
tatawaw

SEQ ID NO: 4 - DNA, 20 bp, Artificial Sequence
  Description of Artificial Sequence Synthetic oligonucleotide
gcaaagcacggagtgcaacc

SEQ ID NO: 5 - DNA, 20 bp, Artificial Sequence
  Description of Artificial Sequence Synthetic oligonucleotide
atgcacgaatagtgctaagc

SEQ ID NO: 6 - DNA, 20 bp, Artificial Sequence
  Description of Artificial Sequence Synthetic oligonucleotide
gcagctagaggttagacctc

SEQ ID NO: 7 - DNA, 20 bp, Artificial Sequence
  Description of Artificial Sequence Synthetic oligonucleotide
gttcctacttcataggattg

SEQ ID NO: 8 - DNA, 20 bp, Artificial Sequence
  Description of Artificial Sequence Synthetic oligonucleotide
aggttaattaacttgccctc

SEQ ID NO: 9 - DNA, 20 bp, Artificial Sequence
  Description of Artificial Sequence Synthetic oligonucleotide
ctgctgtaaacccataccgg

SEQ ID NO: 10 - DNA, 20 bp, Artificial Sequence
  Description of Artificial Sequence Synthetic oligonucleotide
atgcacgaatagtgctaagc

SEQ ID NO: 11 - DNA, 20 bp, Artificial Sequence
  Description of Artificial Sequence Synthetic oligonucleotide
gcaaagcacggagtgcaacc

SEQ ID NO: 12 - DNA, 20 bp, Artificial Sequence
  Description of Artificial Sequence Synthetic oligonucleotide
gcagctagaggttagacctc

SEQ ID NO: 13 - DNA, 20 bp, Artificial Sequence
  Description of Artificial Sequence Synthetic oligonucleotide
gaggttaggggaatccccca

SEQ ID NO: 14 - DNA, 20 bp, Artificial Sequence
  Description of Artificial Sequence Synthetic oligonucleotide
cattttgcggacctggtgtg

SEQ ID NO: 15 - DNA, 20 bp, Artificial Sequence
  Description of Artificial Sequence Synthetic oligonucleotide
ttacgccacggcttgaaagg

SEQ ID NO: 16 - DNA, 20 bp, Artificial Sequence
  Description of Artificial Sequence Synthetic oligonucleotide
ggcgtaccagccactctgaa

SEQ ID NO: 17 - DNA, 20 bp, Artificial Sequence
  Description of Artificial Sequence Synthetic oligonucleotide
aagtttcttcaaacacaatg

SEQ ID NO: 18 - DNA, 20 bp, Artificial Sequence
  Description of Artificial Sequence Synthetic oligonucleotide
gatttcctggcaggacgcgg

SEQ ID NO: 19 - DNA, 20 bp, Artificial Sequence
  Description of Artificial Sequence Synthetic oligonucleotide
gcgcacacctaatattttca

SEQ ID NO: 20 - DNA, 20 bp, Artificial Sequence
  Description of Artificial Sequence Synthetic oligonucleotide
gcagaatagctagagcagca

SEQ ID NO: 21 - DNA, 20 bp, Artificial Sequence
  Description of Artificial Sequence Synthetic oligonucleotide
gtcgccgcagcacccagcgc

SEQ ID NO: 22 - DNA, 20 bp, Artificial Sequence
  Description of Artificial Sequence Synthetic oligonucleotide
agaagagtgcctgcggccag

SEQ ID NO: 23 - DNA, 20 bp, Artificial Sequence
  Description of Artificial Sequence Synthetic oligonucleotide
cgcacgccgcacgcctgcgc

SEQ ID NO: 24 - DNA, 20 bp, Artificial Sequence
  Description of Artificial Sequence Synthetic oligonucleotide
cgatgtcggtgcgcaggcca

SEQ ID NO: 25 - DNA, 20 bp, Artificial Sequence
  Description of Artificial Sequence Synthetic oligonucleotide
ctcgggcgccgcgcagtagc

SEQ ID NO: 26 - DNA, 20 bp, Artificial Sequence
  Description of Artificial Sequence Synthetic oligonucleotide
gcggaagcggccttgcaact

SEQ ID NO: 27 - DNA, 6394 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaagggccgggccatcttcaagcccgaggagctgcggcaggccctgatgcccaccctggaggccctgtaccggcaggaccccgagagcctgcccttccggcagcccgtggacccccagctgctgggcatccccgactacttcgacatcgtgaaatcccccatggacctgagcaccatcaagcggaagctggacaccggccagtaccaggagccctggcagtacgtggacgacatctggctgatgttcaacaacgcctggctgtacaaccggaaaaccagccgggtgtacaagtactgcagcaagctgagcgaggtgttcgagcaggagatcgaccccgtgatgcagagcctgggctactgctgcggccggaagctggagttcagcccccagaccctgtgctgctacggcaagcagctgtgcaccatcccccgggacgccacctactacagctaccagaaccggtaccacttctgcgagaagtgcttcaacgagatccagggcgagagcgtgagcctgggcgacgaccccagccagccccagaccaccatcaacaaggagcagttcagcaagcggaagaacgacaccctggaccccgagctgttcgtggagtgcaccgagtgcggccggaagatgcaccagatctgcgtgctgcaccacgagatcatctggcccgccggcttcgtgtgcgacggctgcctgaagaaatccgcccggacccggaaggagaacaagttcagcgccaagcggctgcccagcacccggctgggcaccttcctggagaaccgggtgaacgacttcctgcggcggcagaaccaccccgagagcggcgaggtgaccgtgcgggtggtgcacgccagcgacaagaccgtggaggtgaagcccggcatgaaggcccggttcgtggacagcggcgagatggccgagagcttcccctaccggaccaaggccctgttcgccttcgaggagatcgacggcgtggacctgtgcttcttcggcatgcacgtgcaggagtacggcagcgactgccccccccccaaccagcggcgggtgtacatcagctacctggacagcgtgcacttcttccggcccaagtgcctgcggaccgccgtgtaccacgagatcctgatcggctacctggagtacgtgaagaagctgggctacaccaccggccacatctgggcctgcccccccagcgagggcgacgactacatcttccactgccacccccccgaccagaagatccccaagcccaagcggctgcaggagtggtacaagaagatgctggacaaggccgtgagcgagcggatcgtgcacgactacaaggacatcttcaagcaggccaccgaggaccggctgaccagcgccaaggagctgccctacttcgagggcgacttctggcccaacgtgctggaggagagcatcaaggagctggagcaggaggaggaggagcggaagcgggaggagaacaccagcaacgagagcaccgacgtgaccaagggcgacagcaagaacgccaagaagaagaacaacaagaaaaccagcaagaacaagagcagcctgagccggggcaacaagaagaagcccggcatgcccaacgtgagcaacgacctgagccagaagctgtacgccaccatggagaagcacaaggaggtgttcttcgtgatccggctgatcgccggccccgccgccaacagcctgccccccatcgtggaccccgaccccctgatcccctgcgacctgatggacggccgggacgccttcctgaccctggcccgggacaagcacctggagttcagcagcctgcggcgggcccagtggagcaccatgtgcatgctggtggagctgcacacccagagccaggacagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - DNA, 6097 bp, Artificial Sequence
  Description of Artificial Sequence Synthetic polynucleotide
aggaaataagagagaaaagaagagtaagaagaaatataagagccaccatggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaagggccgggccgacgccctggacgacttcgacctggacatgctgggcagcgacgccctggacgacttcgacctggacatgctgggcagcgacgccctggacgacttcgacctggacatgctgggcagcgacgccctggacgacttcgacctggacatgctgagcggcggccccaagaagaagcggaaggtgggcagccagtacctgcccgacaccgacgaccggcaccggatcgaggagaagcggaagcggacctacgagaccttcaagagcatcatgaagaaatcccccttcagcggccccaccgacccccggccccccccccggcggatcgccgtgcccagccggagcagcgccagcgtgcccaagcccgccccccagccctaccccttcaccagcagcctgagcaccatcaactacgacgagttccccaccatggtgttccccagcggccagatcagccaggccagcgccctggcccccgcccccccccaggtgctgccccaggcccccgcccccgcccccgcccccgccatggtgagcgccctggcccaggcccccgcccccgtgcccgtgctggcccccggccccccccaggccgtggccccccccgcccccaagcccacccaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccggctggtgaccggcgcccagcggccccccgaccccgcccccgcccccctgggcgcccccggcctgcccaacggcctgctgagcggcgacgaggacttcagcagcatcgccgacatggacttcagcgccctgctgggcagcggcagcggcagccgggacagccgggagggcatgttcctgcccaagcccgaggccggcagcgccatcagcgacgtgttcgagggccgggaggtgtgccagcccaagcggatccggcccttccacccccccggcagcccctgggccaaccggcccctgcccgccagcctggcccccacccccaccggccccgtgcacgagcccgtgggcagcctgacccccgcccccgtgccccagcccctggaccccgcccccgccgtgacccccgaggccagccacctgctggaggaccccgacgaggagaccagccaggccgtgaaggccctgcgggagatggccgacaccgtgatcccccagaaggaggaggccgccatctgcggccagatggacctgagccacccccccccccggggccacctggacgagctgaccaccaccctggagagcatgaccgaggacctgaacctggacagccccctgacccccgagctgaacgagatcctggacaccttcctgaacgacgagtgcctgctgcacgccatgcacatcagcaccggcctgagcatcttcgacaccagcctgttcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 2614 nt, Artificial Sequence
  Description of Artificial Sequence Synthetic polynucleotide
aggaaauaagagagaaaagaagaguaagaagaaauauaagagccaccauggcccccaagaagaagcggaaggugggcauccacggcgugcccgccgcccuggaaccgggcgaaaaaccauacaaauguccggaauguggcaagagcuuuagucgcgccgauaaucugacggaacaccagcgcacccacacccauccacgugccccgaucccgaaaccguuccaauauaaaugcccagaaugcgguaagagcuucagcgacaagaaggaucugacccgccaccaacgcacgcacaccggcgaaaagccguacaaauguccagagugcggcaaaaguuucagccgcgaggauaaccuccauacgcaucagcguacccacaccggugaaaagccauacaaguguccggaaugcggcaaaagcuuuagcacgagcggucaucuggugcgucaccagcguacccauacgggugagaaaccauauaaguguccagagugugguaagagcuuuagccagcuggcgcaucugcgugcccaucaacguacgcauaccggugagaaaccauacaagugcccggagugugguaaaagcuucagcacgagcggcgaacugguucgccaucagcgcacgcacacgggcgaaaagccauauaagugcccggaaugugguaagaguuuuagucagagcggugaucugcgucgucaccaacguacccauaccggcgagaagccaaccggcaagaaaaccagcucgggcggggguggcucagaugcucucgaugauuucgaucucgacaugcucggcuccgacgcucuggacgauuuugaucuggacaugcucggcagcgacgcccucgacgauuucgaucuggauaugcugggcuccgacgcccucgacgacuuugaucucgacaugcucggcagcggcuccggcagccaauaccuccccgacaccgacgauagacauagaauugaggaaaagaggaaaaggacauaugaaaccuuuaaguccaucaugaagaagucccccuuuagcggaccuacagacccuaggccuccucccagaaggaucgccgucccuuccagauccagcgcuuccgugccuaagcccgccccccaacccuaccccuucacauccucccucagcaccaucaacuacgacgaguuucccacaaugguguuccccuccggccagauuagccaagccuccgcucucgcccccgcccccccucaaguccucccucaagcccccgcccccgcccccgcccccgcuauggugagcgcucuggcccaagcccccgcccccgugcccgugcucgcucccggaccuccucaagccguggcuccccccgcccccaaaccuacacaagcuggcgagggcacacuguccgaagcucugcugcagcugcaauucgaugaugaggaucugggagcucugcugggaaacuccaccgaucccgccguguucaccgaucuggccagcgucgacaauagcgaguuccaacagcugcugaaucaaggaauccccguggcuccucauaccaccgagccuaugcucauggaauaccccgaggcuaucacaagacuggugaccggagcucaaagaccccccgaccccgcccccgcuccucugggcgcccccggacugccuaauggacugcugagcggcgacgaggauuucuccuccauugccgauauggacuuuuccgcccuccucggcagcggcagcggaagcagagauucuagagaaggcauguuucugcccaaacccgaagcuggcuccgcuaucuccgacgucuuugagggaagagaggugugucagccuaagagaaucagacccuuucacccucccggcagcccuugggcuaauagaccucuccccgccagccucgccccuacaccuaccggccccgugcaugagcccgucggaagccucacacccgcccccgugccucaaccucucgaccccgcccccgccgucacccccgaagcuucccaucuccucgaagaucccgacgaagaaacaucccaagccgugaaagcccucagagaaauggcugacaccgugaucccucaaaaggaggaagccgcuaucuguggccaaauggaucugagccacccucccccuagaggacaucucgaugagcucacaaccacccucgaaagcaugaccgaagaucucaaucuggauuccccucugacacccgaacucaacgagauucucgacaccuuucugaacgacgagugucugcugcacgccaugcacaucuccaccggccucagcaucuucgauaccagccucuuuggcagcggcuccggcagcggagguggcggaucgggaaagcggcccgccgccaccaagaaggccggccaggccaagaagaagaagggcagcuaccccuacgacgugcccgacuacgccugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 4378 nt, Artificial Sequence
  Description of Artificial Sequence Synthetic polynucleotide
aggaaauaagagagaaaagaagaguaagaagaaauauaagagccaccauggcccccaagaagaagcggaaggugggcauccacggcgugcccgccgccggcagcagcggaucccauaugguugaucugcguacccuggguuauagccagcagcagcaagaaaaaaucaaaccgaaaguucguagcaccguugcacagcaucaugaagcacugguuggucaugguuuuacccaugcacauauuguugcacugagccagcauccggcagcacugggcaccguugcaguuaaauaucaggauaugauugcagcacugccggaagcaacccaugaagcaauuguugguguugguaaacgcggagcuggugcacgugcccuggaagcacugcugaccguugccggugaacugcgugguccgccucugcagcuggauaccggucagcugcugaaaauugcaaaacguggugguguuaccgcaguugaagcaguucaugcauggcguaaugcacugaccggugcaccgcugaaucugacaccggaacagguuguugcaauugccagccaugaugguggcaaacaggcacuggaaaccguucagcgucugcugccgguucugugucaggcacauggucugaccccugaacaggugguggccauugcaagccaugacggcgguaaacaagcccuggaaacagugcagcgccuguuaccggugcugugccaggcccauggcuuaacuccggaacaggugguagcgaucgcaucaaaugguggagguaaacaggccuuagaaaccguacagcgcuuacugccgguguuaugccaggcgcacggccugacgccagaacagguaguggcaaucgccucaaauaaugguggaaaacaggcguuagagacaguccagcgccugcugccuguauuaugucaagcccauggccugaccccagagcaaguuguugcgauugcaaguaacauuggggguaaacaggcacuugagacaguucaacguuuacugccuguacugugccaagcucacggucugacuccggaacaagucgucgcgauugcgaguaacaaugguggcaaacaagcauuagaaacggugcaacgccugcugccaguucuuugccaggcucacgguuuaaccccugagcagguuguagcuauugcgaguaauaauggugguaagcaggcguuggaaacugugcaaagacugcugcccguguugugccaagcacaugguuuaaccccagaacaagucguagcaaucgcaagcaauggcgguggcaagcaagcgcuugaaacaguacagcguuuauuaccgguacuuugucaggcccacggucuuacaccagaacaaguuguggccauagccagucaugauggcggaaagcaggcucuggaaacgguacaacgucuguuaccuguucugugucaagcgcacggauuaacaccugaacaaguaguugccauugcgucaaaugggggaggcaagcaggccuuggagacagugcagagauuacugccaguguugugucaggcucauggccuuacacccgagcaggucguggcaauugcaucuaauaucggcgguaagcaagcuuuagagacuguucagagacugcuuccuguccugugccaggcacacggacuuacgccugagcaagugguugcaaucgccucuaauauaggugguaagcaagcacuggaaacuguccaacgcuuacuuccggugcuuugucaagcacacggcuuaacgccagagcaggucgucgccauagccagccaugacggugguaaacaggcccuugaaacgguccaaagacuucugccgguccuuugccaagcgcaugggcugacaccugagcagguagucgcgauugccucacaugauggugggaagcaggcauuagaaacaguucaaagauuauuaccaguccugugucaggcgcauggguuaaccccagagcagguaguugcaauagcauccaaugguggcggaaaacaagcguuggaaacgguucagcgguuauugccuguuuugugccaggcgcaugguuugacacccgagcaagugguagccauagccucacaugacggggguaaacaagcuuuggagacaguacaacggcugcuuccaguuuuaugucaggcccauggauugacgccugaacaaguugucgcuaucgcaaguaauggcggugguaaacaagcgcuugaaaccguucaacgccuucugccugugcuuugucaggcacauggauuaacacccgaacagguugucgcgauagcuucaaacauugguggucguccggcacuggaaagcauuguugcacagcugagccguccugauccggcacuggcagcacugaccaaugaucaucugguugcacuggcaugucuggguggucgcccugcccuggaugcaguuaaaaaaggucugccgcaugcaccggcacugauuaaacguaccaaucgucguauuccggaacguaccagccaucguguugcuagcggcagcggcggcggcagcggcggcgacgcccuggacgacuucgaccuggacaugcugggcagcgacgcccuggacgacuucgaccuggacaugcugggcagcgacgcccuggacgacuucgaccuggacaugcugggcagcgacgcccuggacgacuucgaccuggacaugcugagcggcggccccaagaagaagcggaaggugggcagccaguaccugcccgacaccgacgaccggcaccggaucgaggagaagcggaagcggaccuacgagaccuucaagagcaucaugaagaaaucccccuucagcggccccaccgacccccggccccccccccggcggaucgccgugcccagccggagcagcgccagcgugcccaagcccgccccccagcccuaccccuucaccagcagccugagcaccaucaacuacgacgaguuccccaccaugguguuccccagcggccagaucagccaggccagcgcccuggcccccgcccccccccaggugcugccccaggcccccgcccccgcccccgcccccgccauggugagcgcccuggcccaggcccccgcccccgugcccgugcuggcccccggccccccccaggccguggccccccccgcccccaagcccacccaggccggcgagggcacccugagcgaggcccugcugcagcugcaguucgacgacgaggaccugggcgcccugcugggcaacagcaccgaccccgccguguucaccgaccuggccagcguggacaacagcgaguuccagcagcugcugaaccagggcauccccguggccccccacaccaccgagcccaugcugauggaguaccccgaggccaucacccggcuggugaccggcgcccagcggccccccgaccccgcccccgccccccugggcgcccccggccugcccaacggccugcugagcggcgacgaggacuucagcagcaucgccgacauggacuucagcgcccugcugggcagcggcagcggcagccgggacagccgggagggcauguuccugcccaagcccgaggccggcagcgccaucagcgacguguucgagggccgggaggugugccagcccaagcggcuccggcccuuccacccccccggcagccccugggccaaccggccccugcccgccagccuggcccccacccccaccggccccgugcacgagcccgugggcagccugacccccgcccccgugccccagccccuggaccccgcccccgccgugacccccgaggccagccaccugcuggaggaccccgacgaggagaccagccaggccgugaaggcccugcgggagauggccgacaccgugaucccccagaaggaggaggccgccaucugcggccagauggaccugagccacccccccccccggggccaccuggacgagcugaccaccacccuggagagcaugaccgaggaccugaaccuggacagcccccugacccccgagcugaacgagauccuggacaccuuccugaacgacgagugccugcugcacgccaugcacaucagcaccggccugagcaucuucgacaccagccuguucagcggcggcaagcggcccgccgccaccaagaaggccggccaggccaagaagaagaagggcagcuaccccuacgacgugcccgacuacgccugagcggccgcuuaauuaagcugccuucugcggggcuugccuucuggccaugcccuucuucucucccuugcaccuguaccucuuggucuuugaauaaagccugaguaggaagucuagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - protein, 2050 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDSGGKRPAATKKAGQAKKKKGSYPYDVPDYA

SEQ ID NO: 32 - protein, 1951 aa, Artificial Sequence
  Description of Artificial Sequence Synthetic polypeptide
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGRADALDDFDLDMLGSDALDDFDLDMLGSDALDDFDLDMLGSDALDDFDLDMLSGGPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFSGGKRPAATKKAGQAKKKKGSYPYDVPDYA

SEQ ID NO: 33 - protein, 790 aa, Artificial Sequence
  Description of Artificial Sequence Synthetic polypeptide
MAPKKKRKVGIHGVPAALEPGEKPYKCPECGKSFSRADNLTEHQRTHTHPRAPIPKPFQYKCPECGKSFSDKKDLTRHQRTHTGEKPYKCPECGKSFSREDNLHTHQRTHTGEKPYKCPECGKSFSTSGHLVRHQRTHTGEKPYKCPECGKSFSQLAHLRAHQRTHTGEKPYKCPECGKSFSTSGELVRHQRTHTGEKPYKCPECGKSFSQSGDLRRHQRTHTGEKPTGKKTSSGGGGSDALDDFDLDMLGSDALDDFDLDMLGSDALDDFDLDMLGSDALDDFDLDMLGSGSGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFGSGSGSGGGGSGKRPAATKKAGQAKKKKGSYPYDVPDYA

SEQ ID NO: 34 - protein, 1378 aa, Artificial Sequence
  Description of Artificial Sequence Synthetic polypeptide
MAPKKKRKVGIHGVPAAGSSGSHMVDLRTLGYSQQQQEKIKPKVRSTVAQHHEALVGHGFTHAHIVALSQHPAALGTVAVKYQDMIAALPEATHEAIVGVGKRGAGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVASGSGGGSGGDALDDFDLDMLGSDALDDFDLDMLGSDALDDFDLDMLGSDALDDFDLDMLSGGPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRLRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFSGGKRPAATKKAGQAKKKKGSYPYDVPDYA

SEQ ID NO: 35 - DNA, 6153 bp, Artificial Sequence
  Description of Artificial Sequence Synthetic polynucleotide
atggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaagggccgggccatcttcaagcccgaggagctgcggcaggccctgatgcccaccctggaggccctgtaccggcaggaccccgagagcctgcccttccggcagcccgtggacccccagctgctgggcatccccgactacttcgacatcgtgaaatcccccatggacctgagcaccatcaagcggaagctggacaccggccagtaccaggagccctggcagtacgtggacgacatctggctgatgttcaacaacgcctggctgtacaaccggaaaaccagccgggtgtacaagtactgcagcaagctgagcgaggtgttcgagcaggagatcgaccccgtgatgcagagcctgggctactgctgcggccggaagctggagttcagcccccagaccctgtgctgctacggcaagcagctgtgcaccatcccccgggacgccacctactacagctaccagaaccggtaccacttctgcgagaagtgcttcaacgagatccagggcgagagcgtgagcctgggcgacgaccccagccagccccagaccaccatcaacaaggagcagttcagcaagcggaagaacgacaccctggaccccgagctgttcgtggagtgcaccgagtgcggccggaagatgcaccagatctgcgtgctgcaccacgagatcatctggcccgccggcttcgtgtgcgacggctgcctgaagaaatccgcccggacccggaaggagaacaagttcagcgccaagcggctgcccagcacccggctgggcaccttcctggagaaccgggtgaacgacttcctgcggcggcagaaccaccccgagagcggcgaggtgaccgtgcgggtggtgcacgccagcgacaagaccgtggaggtgaagcccggcatgaaggcccggttcgtggacagcggcgagatggccgagagcttcccctaccggaccaaggccctgttcgccttcgaggagatcgacggcgtggacctgtgcttcttcggcatgcacgtgcaggagtacggcagcgactgccccccccccaaccagcggcgggtgtacatcagctacctggacagcgtgcacttcttccggcccaagtgcctgcggaccgccgtgtaccacgagatcctgatcggctacctggagtacgtgaagaagctgggctacaccaccggccacatctgggcctgcccccccagcgagggcgacgactacatcttccactgccacccccccgaccagaagatccccaagcccaagcggctgcaggagtggtacaagaagatgctggacaaggccgtgagcgagcggatcgtgcacgactacaaggacatcttcaagcaggccaccgaggaccggctgaccagcgccaaggagctgccctacttcgagggcgacttctggcccaacgtgctggaggagagcatcaaggagctggagcaggaggaggaggagcggaagcgggaggagaacaccagcaacgagagcaccgacgtgaccaagggcgacagcaagaacgccaagaagaagaacaacaagaaaaccagcaagaacaagagcagcctgagccggggcaacaagaagaagcccggcatgcccaacgtgagcaacgacctgagccagaagctgtacgccaccatggagaagcacaaggaggtgttcttcgtgatccggctgatcgccggccccgccgccaacagcctgccccccatcgtggaccccgaccccctgatcccctgcgacctgatggacggccgggacgccttcctgaccctggcccgggacaagcacctggagttcagcagcctgcggcgggcccagtggagcaccatgtgcatgctggtggagctgcacacccagagccaggacagcggcggcaagcggcccgccgccaccaagaaggccggccaggccaagaagaagaagggcagctacccctacgacgtgcccgactacgcctga

SEQ ID NO: 36 - DNA, 5856 bp, Artificial Sequence
  Description of Artificial Sequence Synthetic polynucleotide
atggcccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaatccgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaaaacctacgcccacctgttcgacgacaaggtgatgaagcagctgaagcggcggcggtacaccggctggggccggctgagccggaagctgatcaacggcatccgggacaagcagagcggcaagaccatcctggacttcctgaaatccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggccgccatcgtgccccagagcttcctgaaggacgacagcatcgacaacaaggtgctgacccggagcgacaaggcccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaatccaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaaaaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaatccgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacaagcggcccgccgccaccaagaaggccggccaggccaagaagaagaagggccgggccgacgccctggacgacttcgacctggacatgctgggcagcgacgccctggacgacttcgacctggacatgctgggcagcgacgccctggacgacttcgacctggacatgctgggcagcgacgccctggacgacttcgacctggacatgctgagcggcggccccaagaagaagcggaaggtgggcagccagtacctgcccgacaccgacgaccggcaccggatcgaggagaagcggaagcggacctacgagaccttcaagagcatcatgaagaaatcccccttcagcggccccaccgacccccggccccccccccggcggatcgccgtgcccagccggagcagcgccagcgtgcccaagcccgccccccagccctaccccttcaccagcagcctgagcaccatcaactacgacgagttccccaccatggtgttccccagcggccagatcagccaggccagcgccctggcccccgcccccccccaggtgctgccccaggcccccgcccccgcccccgcccccgccatggtgagcgccctggcccaggcccccgcccccgtgcccgtgctggcccccggccccccccaggccgtggccccccccgcccccaagcccacccaggccggcgagggcaccctgagcgaggccctgctgcagctgcagttcgacgacgaggacctgggcgccctgctgggcaacagcaccgaccccgccgtgttcaccgacctggccagcgtggacaacagcgagttccagcagctgctgaaccagggcatccccgtggccccccacaccaccgagcccatgctgatggagtaccccgaggccatcacccggctggtgaccggcgcccagcggccccccgaccccgcccccgcccccctgggcgcccccggcctgcccaacggcctgctgagcggcgacgaggacttcagcagcatcgccgacatggacttcagcgccctgctgggcagcggcagcggcagccgggacagccgggagggcatgttcctgcccaagcccgaggccggcagcgccatcagcgacgtgttcgagggccgggaggtgtgccagcccaagcggatccggcccttccacccccccggcagcccctgggccaaccggcccctgcccgccagcctggcccccacccccaccggccccgtgcacgagcccgtgggcagcctgacccccgcccccgtgccccagcccctggaccccgcccccgccgtgacccccgaggccagccacctgctggaggaccccgacgaggagaccagccaggccgtgaaggccctgcgggagatggccgacaccgtgatcccccagaaggaggaggccgccatctgcggccagatggacctgagccacccccccccccggggccacctggacgagctgaccaccaccctggagagcatgaccgaggacctgaacctggacagccccctgacccccgagctgaacgagatcctggacaccttcctgaacgacgagtgcctgctgcacgccatgcacatcagcaccggcctgagcatcttcgacaccagcctgttcagcggcggcaagcggcccgccgccaccaagaaggccggccaggccaagaagaagaagggcagctacccctacgacgtgcccgactacgcctga

(19) United States
(12) Patent Application Publication
(10) Pub. No.: US 2022/0348893 A1
(43) Pub. Date: Nov. 3, 2022

(54) METHODS AND COMPOSITIONS FOR MODULATING FRATAXIN EXPRESSION AND TREATING FRIEDRICH'S ATAXIA

(71) Applicant: OMEGA THERAPEUTICS, INC, Cambridge, MA (US)

(72) Inventors: Serena Lunardi, Cambridge, MA (US); Adam Walter Scheidegger, Boston, MA (US); Jesse Jerome Smith, Waltham, MA (US); Jeremiah Dale Farelli, Marblehead, MA (US); Jodi Michelle Kennedy, Dedham, MA (US)

(21) Appl. No.: 17/754,051

(22) PCT Filed: Sep. 23, 2020
(86) PCT No.: PCT/US2020/052101

§ 371 Date: Mar. 22, 2022

Related U.S. Application Data
(60) Provisional application No. 62/904,391, filed on Sep. 23, 2019.

(51) Int. Cl. C12N 9/22; C12N 15/11; A61K 38/46; A61K 31/7105; A61P 25/28; C12N 15/90; C12N 9/10

(52) U.S. Cl. CPC C12N 9/22; C12N 15/11; A61K 38/465; A61K 31/7105; A61P 25/28; C12N 15/907; C12N 9/1029; C12Y 203/01048; C12N 2800/80; C07K 2319/80; C12N 2310/20; C07K 2319/90

(57) ABSTRACT

The present disclosure relates generally to methods and compositions for modulating frataxin (FXN) expression, e.g., to treat Friedreich ataxia (FRDA).

### CROSS-REFERENCE TO RELATED APPLICATIONS

The present application claims priority to and benefit from U.S. provisional application U.S. Ser. No. 62/904,391 (filed Sep. 23, 2019), the contents of which is herein incorporated by reference.

### BACKGROUND

Friedreich ataxia (FRDA) is a lethal autosomal recessive neurodegenerative disorder caused primarily by a homozygous GAA repeat expansion mutation within the first intron of the frataxin (FXN) gene, leading to inhibition of FXN transcription and reduced FXN protein expression. Pathological features of FRDA include: degeneration of large sensory neurons in the dorsal root ganglion (DRG), degenerative atrophy of the spinal cord, hypertrophic cardiomyopathy, and diabetes mellitus. There is a need for methods and compositions that modulate, e.g., increase, expression of FXN in patients suffering from FRDA and/or related symptoms.

### SUMMARY

The present disclosure provides, in part, compositions that modulate, e.g., increase, the expression of the frataxin (FXN) gene. Without wishing to be bound by theory, it is thought that a modulating agent comprising: a targeting moiety that directs the modulating agent to a genomic sequence element (e.g., expression control element) comprised within or operably linked to the FXN gene; and an effector moiety (e.g., comprising an epigenetic modifying moiety) capable of modulating (e.g., increasing) expression of FXN, may be useful to modulate, e.g., increase, expression of FXN.

Accordingly, in some aspects the disclosure is directed, in part, to a modulating agent comprising a targeting moiety that binds to an expression control element of the frataxin (FXN) gene, and an effector moiety comprising an epigenetic modifying moiety capable of modulating, e.g., increasing expression of FXN. In another aspect, the disclosure is directed, in part, to a modulating agent comprising a targeting moiety that binds to an expression control element of the frataxin (FXN) gene, a first effector moiety capable of modulating, e.g., increasing, expression of FXN, and a second effector moiety capable of modulating, e.g., increasing, expression of FXN, wherein the first and second effector moieties are different moieties. In another aspect, the disclosure is directed, in part, to a modulating agent comprising a targeting moiety that binds to an expression control element of the frataxin (FXN) gene, wherein the targeting moiety comprises a Zn Finger molecule, and an effector moiety capable of modulating, e.g., increasing, expression of FXN.

In another aspect, the disclosure is directed, in part, to a nucleic acid molecule encoding a modulating agent, wherein the modulating agent comprises: a targeting moiety, e.g., that binds to an expression control element of the frataxin (FXN) gene, and an effector moiety capable of modulating, e.g., increasing, expression of FXN, wherein the nucleic acid molecule is linear and non-viral. In another aspect, the disclosure is directed, in part, to a nucleic acid molecule encoding a modulating agent described herein (e.g., a nucleic acid molecule that is, is comprised within, or comprises viral nucleic acid, e.g., that is, is comprised within, or comprises a viral vector).

In another aspect, the disclosure is directed, in part, to a recombinant RNA molecule encoding a modulating agent, wherein the modulating agent comprises a targeting moiety that binds to an expression control element of the frataxin (FXN) gene, and an effector moiety capable of modulating, e.g., increasing, expression of FXN.

In another aspect, the disclosure is directed, in part, to a viral vector comprising a nucleic acid or recombinant RNA molecule described herein.

In another aspect, the disclosure is directed, in part, to a nanoparticle (e.g., a lipid nanoparticle (LNP)) comprising a nucleic acid, e.g., a recombinant RNA, encoding a modulating agent, the modulating agent comprising: a targeting moiety that binds to an expression control element of the frataxin (FXN) gene, and an effector moiety capable of modulating, e.g., increasing, expression of FXN.

The present disclosure further provides, in part, methods of modulating, e.g., increasing, the expression of the frataxin (FXN) gene, e.g., in a patient in need thereof (e.g., a patient with FRDA). Without wishing to be bound by theory, it is thought that administering a modulating agent comprising: a targeting moiety that directs the modulating agent to a genomic sequence element (e.g., expression control element) comprised within or operably linked to the FXN gene; and an effector moiety (e.g., comprising an epigenetic modifying moiety) capable of modulating (e.g., increasing) expression of FXN, may modulate, e.g., increase, expression of FXN and/or increase the levels of FXN protein in a patient in need thereof.

Accordingly, in some aspects the disclosure is directed, in part, to a method of increasing frataxin (FXN) expression in a cell, comprising contacting a cell with a modulating agent, the modulating agent comprising: a targeting moiety that binds to an expression control element of the frataxin (FXN) gene, and an effector moiety capable of modulating, e.g., increasing, expression of FXN, thereby increasing FXN expression in the cell, wherein FXN expression increases for at least 1, 2, 3, 4, 5, 6, 7, 8, 9, or 10 weeks (and optionally, permanently). In another aspect, the disclosure is directed, in part, to a method of increasing frataxin (FXN) expression in a cell, comprising contacting a cell with a modulating agent, the modulating agent comprising: a targeting moiety that binds to an expression control element of the frataxin (FXN) gene, and an effector moiety capable of modulating, e.g., increasing, expression of FXN, thereby increasing FXN expression in the cell, wherein the cell comprises a FXN allele comprising a GAA expansion of at least 44 copies, wherein after treatment with the modulating agent the FXN allele is expressed at a level of at least 1.5× (i.e., 1.5 times) the expression level of a similar cell not contacted with the modulating agent. In another aspect, the disclosure is directed, in part, to a method of increasing frataxin (FXN) expression in a cell, comprising contacting a cell with a modulating agent described herein.

The present disclosure further provides, in part, a human cell comprising one or two frataxin (FXN) alleles comprising a GAA expansion of at least 44 copies, wherein the FXN allele is expressed at a higher level than the level of FXN expression in a cell that has not been treated with a modulating agent capable of modulating FXN expression (e.g., a modulating agent described herein). Without wishing to be bound by theory, it is thought that a cell treated with a modulating agent described herein may exhibit increased FXN expression that persists over an extended duration, e.g., that exceeds the time period in which the modulating agent is/was present in the cell. In some embodiments, the FXN allele is expressed at a level of at least 1.5× (i.e., 1.5 times), 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, or 5× a reference level, wherein the reference level is the level of FXN expression in a cell that has not been treated with a modulating agent capable of modulating FXN expression (e.g., a modulating agent described herein). In some embodiments, the cell is a muscle cell (e.g., a muscle cell in the heart, e.g., a cardiomyocyte) or a neuronal cell (e.g., a cell of the central nervous system or a cell of the spine, e.g., a cell (e.g., neuron) of the dorsal root ganglia (DRG)). In some embodiments, the neuronal cell is a glutamatergic cortical neuron.

Additional features of any of the aforesaid methods or compositions include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Sep. 23, 2019. When one gene or protein references a plurality of sequence accession numbers, all of the sequence variants are encompassed.

### ENUMERATED EMBODIMENTS

1. A modulating agent comprising:

a targeting moiety that binds to an expression control element of the frataxin (FXN) gene, and

an effector moiety comprising an epigenetic modifying moiety capable of modulating, e.g., increasing expression of FXN.

2. A modulating agent comprising:

a targeting moiety that binds to an expression control element of the frataxin (FXN) gene,

a first effector moiety capable of modulating, e.g., increasing, expression of FXN, and

a second effector moiety capable of modulating, e.g., increasing, expression of FXN,

wherein the first and second effector moieties are different moieties.

3. A modulating agent comprising:

a targeting moiety that binds to an expression control element of the frataxin (FXN) gene, wherein the targeting moiety comprises a Zn Finger molecule, and

an effector moiety capable of modulating, e.g., increasing, expression of FXN.

4. A nucleic acid encoding a modulating agent, wherein the modulating agent comprises:

a targeting moiety that binds to an expression control element of the frataxin (FXN) gene, and

an effector moiety capable of modulating, e.g., increasing, expression of FXN,

wherein the nucleic acid molecule is linear and non-viral.

5. A nucleic acid encoding a modulating agent of any of embodiments 1-4.
6. A recombinant RNA encoding a modulating agent, wherein the modulating agent comprises:

a targeting moiety that binds to an expression control element of the frataxin (FXN) gene, and

an effector moiety capable of modulating, e.g., increasing, expression of FXN.

7. A nanoparticle (e.g., a lipid nanoparticle (LNP)) comprising a nucleic acid, e.g., a recombinant RNA, encoding a modulating agent, the modulating agent comprising:

a targeting moiety that binds to an expression control element of the frataxin (FXN) gene, and

an effector moiety capable of modulating, e.g., increasing, expression of FXN.

8. A viral vector comprising the nucleic acid or recombinant RNA molecule of any of embodiments 4-6.
9. A method of increasing frataxin (FXN) expression in a cell, comprising:

contacting a cell with a modulating agent, the modulating agent comprising:
a targeting moiety that binds to an expression control element of the frataxin (FXN) gene, and
an effector moiety capable of modulating, e.g., increasing, expression of FXN, thereby increasing FXN expression in the cell,

wherein FXN expression increases for at least 1, 2, 3, 4, 5, 6, 7, 8, 9, or 10 weeks (and optionally, permanently).

10. A method of increasing frataxin (FXN) expression in a cell, comprising:

contacting a cell with a modulating agent, the modulating agent comprising:
a targeting moiety that binds to an expression control element of the frataxin (FXN) gene, and
an effector moiety capable of modulating, e.g., increasing, expression of FXN, thereby increasing FXN expression in the cell,

wherein the cell comprises a FXN allele comprising a GAA expansion of at least 44 copies, wherein after treatment with the modulating agent the FXN allele is expressed at a level of at least 1.5× (i.e., 1.5 times) the expression level of a similar cell not contacted with the modulating agent.

11. A method of increasing frataxin (FXN) expression in a cell, comprising:

contacting a cell with the modulating agent, nucleic acid, recombinant RNA, nanoparticle, or viral vector of any of embodiments 1-8,

thereby increasing FXN expression in the cell.

12. A modulating agent comprising:

a targeting moiety that binds to an expression control element of the frataxin (FXN) gene, wherein the expression control element does not comprise the promoter or transcription start site of FXN, and

an effector moiety capable of modulating, e.g., increasing, expression of FXN.

13. A modulating agent comprising:

a targeting moiety that binds to a nucleic acid sequence of an expression control element of the frataxin (FXN) gene, wherein the nucleic acid sequence position nearest to the TSS is: i) about 150 bases upstream of the TSS; or ii) about 50 bases downstream of the TSS; and

an effector moiety capable of modulating, e.g., increasing, expression of FXN.

14. A human cell comprising:

a frataxin (FXN) allele comprising a GAA expansion of at least 44 copies, wherein the FXN allele is expressed at a level of at least 1.5× (i.e., 1.5 times), 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, or 5× a reference level, wherein the reference level is the level of FXN expression in a cell that has not been treated with a modulating agent capable of modulating FXN expression (e.g., a modulating agent of any preceding claim),

wherein the cell is a muscle cell, neuronal cell, or a cell of the dorsal root ganglia.

15. A human cell comprising:

two frataxin (FXN) alleles each comprising a GAA expansion of at least 44 copies, wherein each allele is expressed at a level of at least 1.5× (i.e., 1.5 times), 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, or 5× a reference level, wherein the reference level is the level of FXN expression in a cell that has not been treated with a modulating agent capable of modulating FXN expression (e.g., a modulating agent of any preceding claim),

wherein the cell is a muscle cell, neuronal cell, or a cell of the dorsal root ganglia.

16. The modulating agent, nucleic acid, recombinant RNA, nanoparticle, viral vector, or method of any of embodiments 1-13, wherein the effector moiety comprises an epigenetic modifying moiety.
17. The modulating agent, nucleic acid, recombinant RNA, nanoparticle, viral vector, or method of any of embodiments 1-13 or 16, wherein the epigenetic modifying moiety comprises a histone methyltransferase, a DNA demethylase, a histone acetyltransferase, or a functional fragment or variant of any thereof.
18. The modulating agent, nucleic acid, recombinant RNA, nanoparticle, viral vector, or method of any of embodiments 1-13, 16, or 17 wherein the effector moiety comprises a DNA demethylase or functional fragment or variant thereof, e.g., a protein chosen from TET1, TET2, TET3, or TDG, or a functional variant or fragment of any thereof.
19. The modulating agent, nucleic acid, recombinant RNA, nanoparticle, viral vector, or method of any of embodiments 1-13 or 16-18, wherein the effector moiety comprises a histone methyltransferase or functional fragment or variant thereof, e.g., a protein chosen from DOT1L, PRDM9, PRMT1, PRMT2, PRMT3, PRMT4, PRMT5, NSD1, NSD2, NSD3, or a functional variant or fragment of any thereof.
20. The modulating agent, nucleic acid, recombinant RNA, nanoparticle, viral vector, or method of any of embodiments 1-13 or 16-19, wherein the effector moiety comprises a histone acetyltransferase or functional fragment or variant thereof, e.g., a protein chosen from p300, CREB-binding protein (CBP), or functional fragment or variant thereof.
21. The modulating agent, nucleic acid, recombinant RNA, nanoparticle, viral vector, or method of any of embodiments 1-13 or 16-20, wherein the effector moiety comprises a transcriptional activator or functional fragment or variant thereof, e.g., a protein chosen from VP16, VP64, VP160, or VPR.
22. The modulating agent, nucleic acid, recombinant RNA, nanoparticle, viral vector, or method of any of embodiments 1-13 or 16-21, wherein the effector moiety comprises VPR or a functional fragment or variant thereof.
23. The modulating agent, nucleic acid, recombinant RNA, nanoparticle, viral vector, or method of any of embodiments 1-13 or 16-22, wherein the effector moiety comprises p300 or a functional fragment or variant thereof.
24. The modulating agent, nucleic acid, recombinant RNA, nanoparticle, viral vector, or method of any of embodiments 1-13 or 16-23, wherein the effector moiety comprises p65 or a functional fragment or variant thereof.
25. The modulating agent, nucleic acid, recombinant RNA, nanoparticle, viral vector, or method of any of embodiments 1-13 or 16-24, wherein the effector moiety comprises RTA or a functional fragment or variant thereof.
26. The modulating agent, nucleic acid, recombinant RNA, nanoparticle, viral vector, or method of any of embodiments 1-13 or 16-25, wherein the effector moiety comprises 1, 2, or all of a DNA demethylase, an acetyltransferase, or a transcriptional activator, or functional fragment of any thereof.
27. The modulating agent, nucleic acid, recombinant RNA, nanoparticle, viral vector, or method of any of embodiments 1-13 or 16-26, wherein the targeting moiety comprises a Cas9 molecule.
28. The modulating agent, nucleic acid, recombinant RNA, nanoparticle, viral vector, or method of embodiment 27, wherein the Cas9 molecule comprises a Cas9 protein from Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.
29. The modulating agent, nucleic acid, recombinant RNA, nanoparticle, viral vector, or method of either embodiments 27 or 28, wherein the Cas9 molecule comprises a Cas9 protein substantially lacking nuclease activity, e.g., dCas9, e.g., comprising inactive RuvC and/or HNH domains.
30. The modulating agent, nucleic acid, recombinant RNA, nanoparticle, viral vector, or method of any of embodiments 27-29 wherein the Cas9 molecule comprises (e.g., is noncovalently bound to) a gRNA, e.g., an sgRNA, wherein the gRNA binds to the expression control element.
31. The modulating agent, nucleic acid, recombinant RNA, nanoparticle, viral vector, or method of embodiment 30, wherein the gRNA comprises a nucleic acid sequence selected from any of SEQ ID NOs: 4-26, or a sequence with at least 80, 85, 90, 95, or 99% identity to any of SEQ ID NOs: 4-26.
32. The modulating agent, nucleic acid, recombinant RNA, nanoparticle, viral vector, or method of any of embodiments 1-13 or 16-26, wherein the targeting moiety comprises a TAL effector molecule.
33. The modulating agent, nucleic acid, recombinant RNA, nanoparticle, viral vector, or method of embodiment 32, wherein the TAL effector molecule comprises 5, 6, 7, 8, 9, 10, 11, 12, 13, 14, 15, 16, 17, 18, 19, or 20 TAL effector DNA binding domains (e.g., 5, 6, 7, 8, 9, 10, 11, 12, 13, 14, 15, 16, 17, 18, 19, or 20 repeat variable diresidues (RVDs)).
34. The modulating agent, nucleic acid, recombinant RNA, nanoparticle, viral vector, or method of any of embodiments 1-13 or 16-26, wherein the targeting moiety comprises a Zn Finger molecule.
35. The modulating agent, nucleic acid, recombinant RNA, nanoparticle, viral vector, or method of any of embodiments 1-13 or 16-34, wherein the expression control element comprises an enhancer or promoter or portion thereof operably linked to the FXN gene.
36. The modulating agent, nucleic acid, recombinant RNA, nanoparticle, viral vector, or method of any of embodiments 1-13 or 16-35, wherein the expression control element comprises an anchor sequence operably linked to an anchor sequence mediated conjunction comprising, wholly or in part, the FXN gene.
37. The modulating agent, nucleic acid, recombinant RNA, nanoparticle, viral vector, or method of any of embodiments 1-13 or 16-36, wherein the targeting moiety binds to a nucleic acid sequence comprising the transcription start site (TSS) of the FXN gene.
38. The modulating agent, nucleic acid, recombinant RNA, nanoparticle, viral vector, or method of any of embodiments 1-13 or 16-37, wherein the targeting moiety binds to a nucleic acid sequence that is no more than 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10, 9, 8, 7, 6, 5, 4, 3, 2, or 1 nucleotides upstream or downstream from the transcription start site (TSS) of the FXN gene (and optionally at least 1, 2, 3, 4, 5, 6, 7, 8, 9, 10, 20, 30, 40, 50, 60, 70, 80, or 90 nucleotides upstream or downstream).
39. The modulating agent, nucleic acid, recombinant RNA, nanoparticle, viral vector, or method of any of embodiments 1-13 or 16-37, wherein the targeting moiety binds to a nucleic acid sequence that is about 50-150, 50-70, 70-90, 90-110, 110-130, or 130-150 nucleotides upstream from the transcription start site (TSS) of the FXN gene.
40. The modulating agent, nucleic acid, recombinant RNA, nanoparticle, viral vector, or method of any of embodiments 1-13 or 16-39, wherein the targeting moiety comprises a Zn Finger molecule that comprises 2, 3, 4, 5, or 6 Zn finger proteins.
41. The modulating agent, nucleic acid, recombinant RNA, nanoparticle, viral vector, or method of any of embodiments 1-13 or 16-40, wherein the targeting moiety binds to a nucleic acid sequence selected from a sequence denoted by genomic coordinates of Table 3.
42. The modulating agent, nucleic acid, recombinant RNA, nanoparticle, viral vector, or method of any of embodiments 1-13, 16-31, 35-39, or 41, wherein the targeting moiety comprises a Cas9 molecule, e.g., a dCas9 molecule, and the effector moiety comprises p300 or a functional fragment or variant thereof.
43. The modulating agent, nucleic acid, recombinant RNA, nanoparticle, viral vector, or method of any of embodiments 1-13, 16-31, 35-39, or 41, wherein the targeting moiety comprises a Cas9 molecule, e.g., a dCas9 molecule, and the effector moiety comprises VP64 or a functional fragment or variant thereof.
44. The modulating agent, nucleic acid, recombinant RNA, nanoparticle, viral vector, or method of any of embodiments 1-13, 16-31, 35-39, or 41,

wherein the targeting moiety comprises an enzymatically inactive Cas nuclease, e.g., a dCas9 molecule, and the effector moiety comprises p300 or a functional fragment or variant thereof.

45. The modulating agent, nucleic acid, recombinant RNA, nanoparticle, viral vector, or method of any of embodiments 1-13, 16-31, 35-39, or 41,

wherein the targeting moiety comprises an enzymatically inactive Cas nuclease, e.g., a dCas9 molecule, and the effector moiety comprises VP64 or a functional fragment or variant thereof.

46. The modulating agent, nucleic acid, recombinant RNA, nanoparticle, viral vector, or method of any of embodiments 1-13, 16-31, 35-39, or 41,

wherein the targeting moiety comprises an enzymatically inactive Cas nuclease, e.g., a dCas9 molecule, and the effector moiety comprises VPR or a functional fragment or variant thereof.

47. The modulating agent, nucleic acid, recombinant RNA, nanoparticle, viral vector, or method of any of embodiments 1-13, 16-31, 35-39, or 41,

wherein the targeting moiety comprises an enzymatically inactive Cas nuclease, e.g., a dCas9 molecule, and the effector moiety comprises VP64 or a functional fragment or variant thereof, p65 or a functional fragment or variant thereof, and RTA or a functional fragment or variant thereof.

48. The modulating agent, nucleic acid, recombinant RNA, nanoparticle, viral vector, or method of any of embodiments 1-13, 16-31, 35-39, or 41,

wherein the targeting moiety comprises a TAL effector molecule (e.g., wherein the TAL effector molecule binds upstream of the FXN gene TSS, e.g., about 50-150 nucleotides upstream, e.g., about 100 nucleotides upstream), and the effector moiety comprises VPR or a functional fragment or variant thereof.

49. The modulating agent, nucleic acid, recombinant RNA, nanoparticle, viral vector, or method of any of embodiments 1-13, 16-31, 35-39, or 41, wherein the targeting moiety comprises a TAL effector molecule molecule (e.g., wherein the TAL effector molecule binds upstream of the FXN gene TSS, e.g., about 50-150 nucleotides upstream, e.g., about 100 nucleotides upstream), and the effector moiety comprises VP64 or a functional fragment or variant thereof, p65 or a functional fragment or variant thereof, and RTA or a functional fragment or variant thereof.

50. The modulating agent, nucleic acid, recombinant RNA, nanoparticle, viral vector, or method of any of embodiments 1-13, 16-31, 35-39, or 41,

wherein the targeting moiety comprises a Zn finger molecule (e.g., wherein the Zn finger molecule binds upstream of the FXN gene TSS, e.g., about 50-150 nucleotides upstream, e.g., about 100 nucleotides upstream), and the effector moiety comprises VPR or a functional fragment or variant thereof.

51. The modulating agent, nucleic acid, recombinant RNA, nanoparticle, viral vector, or method of any of embodiments 1-13, 16-31, 35-39, or 41,

wherein the targeting moiety comprises a Zn finger molecule molecule (e.g., wherein the Zn finger molecule binds upstream of the FXN gene TSS, e.g., about 50-150 nucleotides upstream, e.g., about 100 nucleotides upstream), and the effector moiety comprises VP64 or a functional fragment or variant thereof, p65 or a functional fragment or variant thereof, and RTA or a functional fragment or variant thereof.

52. The modulating agent, nucleic acid, recombinant RNA, nanoparticle, viral vector, or method of any of embodiments 1-13 or 16-51, wherein the modulating agent comprises or is a fusion molecule.
53. The modulating agent, nucleic acid, recombinant RNA, nanoparticle, viral vector, or method of any of embodiments 1-13 or 16-52, wherein the modulating agent comprises an amino acid sequence selected from any of SEQ ID NOs: 304-309, or an amino acid sequence with at least 80, 85, 90, 95, 96, 97, 98, or 99% identity thereto.
54. The modulating agent, nucleic acid, recombinant RNA, nanoparticle, viral vector, or method of embodiment 52 or 53, wherein the fusion molecule comprises the targeting moiety and effector moiety covalently linked, e.g., by a peptide bond, e.g., as part of a single polypeptide chain.
55. The modulating agent, nucleic acid, recombinant RNA, nanoparticle, viral vector, or method of any of embodiments 1-13 or 16-54, wherein the modulating agent comprises or is a conjugate.
56. The modulating agent, nucleic acid, recombinant RNA, nanoparticle, viral vector, or method of embodiment 55, wherein the conjugate comprises the targeting moiety and effector moiety covalently linked, e.g., by a non-peptide bond.
57. The modulating agent, nucleic acid, recombinant RNA, nanoparticle, viral vector, or method of any of embodiments 1-13 or 16-56, wherein the modulating agent further comprises an additional moiety.
58. The modulating agent, nucleic acid, recombinant RNA, nanoparticle, viral vector, or method of embodiment 57, wherein the additional moiety comprises a purification tag (e.g., a moiety that aids in purification of the modulating agent), a bioavailability or pharmacokinetic moiety (e.g., a moiety that increases the bioavailability or modulates the pharmacokinetic properties of the modulating agent), a solubility moiety (e.g., a moiety that increases the solubility, e.g., physiological solubility, of the modulating agent), a detection moiety (e.g., a moiety that aids in detecting and/or quantifying the presence or level of the modulating agent, e.g., a fluorescent moiety or fluorophore), a multimerization moiety (e.g., a moiety that promotes multimerization (e.g., dimerization, trimerization, or tetramerization) of the modulating agent), or an association moiety (e.g., a moiety that allows the modulating agent to associate with a structure, e.g., a membrane or lab testing device (e.g., plate or tube wall)).
59. A complex comprising a modulating agent of any of embodiments 1-3, 12, 13, or 16-58 and a nucleic acid sequence comprising the expression control sequence of the FXN gene.
60. A cell comprising the modulating agent, nucleic acid, recombinant RNA, nanoparticle, or viral vector of any of embodiments 1-8, 12, 13, or 16-58.
61. A cell comprising a nucleic acid encoding the modulating agent of any of embodiments 1-3, 12, 13, or 16-58.
62. A method of delivering a modulating agent, nucleic acid, recombinant RNA, nanoparticle, or viral vector of any of embodiments 1-8, 12, 13, or 16-58 to a cell, comprising contacting the cell with the modulating agent, nucleic acid, recombinant RNA, nanoparticle, or viral vector, thereby delivering the modulating agent, nucleic acid, recombinant RNA, nanoparticle, or viral vector to the cell.
63. The method of embodiment 62, which further comprises contacting the cell with one or more (e.g., 2 or 3) gRNA(s) that bind an expression control element of the FXN gene, or DNA encoding the gRNA(s).
64. A method of modulating, e.g., increasing, transcription of the frataxin (FXN) gene, comprising:

contacting a cell with the modulating agent, nucleic acid, recombinant RNA, nanoparticle, or viral vector of any of embodiments 1-8, 12, 13, or 16-58,

thereby modulating, e.g., increasing, expression of the FXN gene.

65. The method of any of embodiments 62-64, wherein contacting occurs in vivo, in vitro, or ex vivo.
66. A method of treating a patient having Friedrich's Ataxia (FRDA), comprising:

administering a modulating agent, nucleic acid, recombinant RNA, nanoparticle, or viral vector of any of embodiments 1-8, 12, 13, or 16-58 to the patient,

thereby treating the patient.

67. The method of embodiment 66, wherein administration comprises intravenous or intrathecal administration.
68. The method of any of embodiments 62-67, wherein the method increases FXN levels in blood (e.g., whole blood) by at least 10, 20, 30, 40, 50, 60, 70, 80, 90, 100, 120, 140, 160, 180, 200, 300, or 400% relative to FXN levels in blood (e.g., whole blood) in the absence of the modulating agent, nucleic acid, recombinant RNA, nanoparticle, or viral vector (e.g., as measured by the methods of Deutsch et al or Oglesbee et al).
69. The method of any of embodiments 62-68, wherein the method lessens or eliminates at least one symptom of FDRA, e.g., a symptom selected from ataxia, dysarthria, muscle weakness, spasticity (e.g., lower limb spasticity), scoliosis, bladder dysfunction, reflex dysfunction, loss of position and/or vibration sense, cardiomyopathy, or diabetes mellitus.
70. The method of either of embodiments 68 or 69, wherein the level of FXN in blood (e.g., whole blood) is increased for at least 1, 2, 3, 4, 5, 6, 7, 8, 9, or 10 weeks, or at least 1, 2, 3, 4, 5, 6, 7, 8, 9, 10, 11, or 12 months, or at least 1, 2, 3, 4, or 5 years.
71. The method of any of embodiments 62-69, wherein the method increases FXN levels in blood (e.g., whole blood) for at least 12, 18, 24, 30, 36, 42, 48, 54, 60, 66, 72, 78, 84, 90, or 96 hours.
72. A method of increasing frataxin (FXN) expression in a cell, comprising:

contacting a cell with a modulating agent of any preceding embodiment,

thereby increasing FXN expression in the cell,

wherein FXN expression increases for at least 12, 18, 24, 30, 36, 42, 48, 54, 60, 66, 72, 78, 84, 90, or 96 (and optionally, permanently).

### Definitions

A, an, the: As used herein, the singular forms “a,” “an” and “the” include plural referents unless the context clearly dictates otherwise.
Agent: As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some embodiments, the term “agent” may refer to a compound or entity that is or comprises a polymer; in some embodiments, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.
Anchor Sequence: The term “anchor sequence” as used herein, refers to a sequence recognized by a conjunction nucleating polypeptide (e.g., a nucleating polypeptide) that binds sufficiently to form an anchor sequence-mediated conjunction. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments of the present disclosure, technologies are provided that may specifically target a particular anchor sequence or anchor sequences, without targeting other anchor sequences (e.g., sequences that may contain a conjunction nucleating polypeptide (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated.
Anchor sequence-mediated conjunction: The term “anchor sequence-mediated conjunction” (also abbreviated ASMC) as used herein, refers to a DNA structure that occurs and/or is maintained via physical interaction or binding of at least two anchor sequences in the DNA by one or more proteins, such as nucleating polypeptides, or one or more proteins and/or a nucleic acid entity (such as RNA or DNA), that bind the anchor sequences to enable spatial proximity and functional linkage between the anchor sequences.
Associated with: Two events or entities are “associated” with one another, as that term is used herein, if presence, level, function, and/or form of one is correlated with that of the other. For example, in some embodiments, a particular entity (e.g., polypeptide, genetic signature, metabolite, microbe, etc.) is considered to be associated with a particular disease, disorder, or condition, if its presence, level, function, and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a target gene is “associated with” an anchor sequence-mediated conjunction if modulation (e.g., disruption) of the anchor sequence-mediated conjunction causes an alteration in expression (e.g., transcription) of the target gene. For example, in some embodiments, modulation (e.g., disruption) of an anchor sequence-mediated conjunction causes an enhancing or silencing/repressor sequence to associate with or become unassociated with a target gene, thereby altering expression of the target gene. In some embodiments, a target gene is associated with an ASMC if the target gene is situated within or partially within the ASMC.
Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).
Effector moiety: As used herein, the term “effector moiety” refers to a domain that is capable of altering the expression of a target gene (e.g., FXN) when localized to an appropriate site in the nucleus of a cell. In some embodiments, an effector moiety recruits components of the transcription machinery. In some embodiments, an effector moiety inhibits recruitment of components of transcription factors or expression repressing factors. In some embodiments, an effector moiety comprises an epigenetic modifying moiety (e.g., epigenetically modifies a target DNA sequence).
Epigenetic modifying moiety: As used herein, “epigenetic modifying moiety” refers to a domain that alters: i) the structure, e.g., two dimensional structure, of chromatin; and/or ii) an epigenetic marker (e.g., one or more of DNA methylation, histone methylation, histone acetylation, histone sumoylation, histone phosphorylation, and RNA-associated silencing), when the epigenetic modifying moiety is appropriately localized to a nucleic acid (e.g., by a targeting moiety). In some embodiments, an epigenetic modifying moiety comprises an enzyme, or a functional fragment or variant thereof, that affects (e.g., increases or decreases the level of) one or more epigenetic markers. In some embodiments, an epigenetic modifying moiety comprises a DNA methyltransferase, a histone methyltransferase, CREB-binding protein (CBP), or a functional fragment of any thereof.
Expression control sequence: As used herein, the term “expression control sequence” as used herein, refers to a nucleic acid sequence that increases or decreases transcription of a gene, and includes (but is not limited to) a promoter and an enhancer. An “enhancing sequence” refers to a subtype of expression control sequence and increases the likelihood of gene transcription. A “silencing or repressor sequence” refers to a subtype of expression control sequence and decreases the likelihood of gene transcription.
Fusion Molecule: As used herein, the term “fusion molecule” refers to a compound comprising two or more moieties, e.g., a targeting moiety and an effector moiety, that are covalently-linked. A fusion molecule and its moieties may comprise any combination of polypeptide, nucleic acid, glycan, small molecule, or other components described herein (e.g., a targeting moiety may comprise a nucleic acid and an effector moiety may comprise a polypeptide). In some embodiments, a fusion molecule is a fusion protein, e.g., comprising one or more polypeptide domains covalently linked via peptide bonds. In some embodiments, a fusion molecule is a conjugate molecule that comprises a targeting moiety and effector moiety that are linked by a covalent bond other than a peptide bond or phosphodiester bond (e.g., a targeting moiety that comprises a nucleic acid and an effector moiety comprising a polypeptide linked by a covalent bond other than a peptide bond or phosphodiester bond). In some embodiments, a modulating agent is or comprises a fusion molecule.
Genomic complex: As used herein, the term “genomic complex” is a complex that brings together two genomic sequence elements that are spaced apart from one another on one or more chromosomes, via interactions between and among a plurality of protein and/or other components (potentially including the genomic sequence elements). In some embodiments, the genomic sequence elements are anchor sequences to which one or more protein components of the complex binds. In some embodiments, a genomic complex may be an anchor sequence mediated conjunction (ASMC). In some embodiments, a genomic complex comprises one or more ASMCs. In some embodiments, a genomic sequence element may be or comprise an anchor sequence (e.g., a CTCF binding motif), a promoter and/or an enhancer. In some embodiments, a genomic sequence element includes at least one or both of a promoter and/or an enhancer. In some embodiments, genomic complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., enhancer RNA (eRNA)), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration that it does not adopt when the complex is not formed.
Moiety: As used herein, the term “moiety” refers to a defined chemical group or entity with a particular structure and/or or activity, as described herein.
Modulating agent: As used herein, the term “modulating agent” refers to an agent comprising one or more targeting moieties and one or more effector moieties that is capable of altering (e.g., increasing or decreasing) expression of a target gene, e.g., FXN.
Nucleating polypeptide: As used herein, the term “nucleating polypeptide” or “conjunction nucleating polypeptide” as used herein, refers to a protein that associates with an anchor sequence directly or indirectly and may interact with one or more conjunction nucleating polypeptides (that may interact with an anchor sequence or other nucleic acids) to form a dimer (or higher order structure) comprised of two or more such conjunction nucleating polypeptides, which may or may not be identical to one another. When conjunction nucleating polypeptides associated with different anchor sequences associate with each other so that the different anchor sequences are maintained in physical proximity with one another, the structure generated thereby is an anchor-sequence-mediated conjunction. That is, the close physical proximity of a nucleating polypeptide-anchor sequence interacting with another nucleating polypeptide-anchor sequence generates an anchor sequence-mediated conjunction (e.g., in some cases, a DNA loop), that begins and ends at the anchor sequence. As those skilled in the art, reading the present specification will immediately appreciate, terms such as “nucleating polypeptide”, “nucleating molecule”, “nucleating protein”, “conjunction nucleating protein”, may sometimes be used to refer to a conjunction nucleating polypeptide. As will similarly be immediately appreciated by those skilled in the art reading the present specification, an assembles collection of two or more conjunction nucleating polypeptides (which may, in some embodiments, include multiple copies of the same agent and/or in some embodiments one or more of each of a plurality of different agents) may be referred to as a “complex”, a “dimer” a “multimer”, etc.
Operably linked: As used herein, the phrase “operably linked” refers to a juxtaposition wherein the components described are in a relationship permitting them to function in their intended manner. An expression control sequence “operably linked” to a functional element, e.g., gene, is associated in such a way that expression and/or activity of the functional element, e.g., gene, is achieved under conditions compatible with the expression control sequence. In some embodiments, “operably linked” expression control sequences are contiguous (e.g., covalently linked) with coding elements, e.g., genes, of interest; in some embodiments, operably linked expression control sequences act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other.
Pharmaceutical composition: As used herein, the term “pharmaceutical composition” refers to an active agent (e.g., a modulating agent, e.g., a disrupting agent), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.
Proximal: As used herein, “proximal” refers to a closeness of two sites, e.g., nucleic acid sites, such that binding of an expression repressor at the first site and/or modification of the first site by an expression repressor will produce the same or substantially the same effect as binding and/or modification of the other site. For example, a DNA-targeting moiety may bind to a first site that is proximal to an enhancer (the second site), and the repressor domain associated with said DNA-targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, or proximal to an anchor sequence is less than 5000, 4000, 3000, 2000, 1000, 900, 800, 700, 600, 500, 400, 300, 200, 100, 50, or 25 base pairs from the target gene (e.g., an exon, intron, or splice site within the target gene), transcription control element, or anchor sequence (and optionally at least 20, 25, 50, 100, 200, or 300 base pairs from the target gene (e.g., an exon, intron, or splice site within the target gene), transcription control element, or anchor sequence).
Specific: As used herein, the term “specific” refers to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In some embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).
Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.
Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence. In some embodiments, target binding is direct binding; in some embodiments, target binding may be indirect binding. In some embodiments, a modulating agent targets a genomic complex, e.g., ASMC, by binding to a component (e.g., polypeptide, nucleic acid, and/or genomic sequence element) of the genomic complex, e.g., ASMC.
Target gene: As used herein, the term “target gene” means a gene that is targeted for modulation, e.g., modulation of expression of the gene or modulation of an epigenetic marker associated with the gene. In some embodiments, a target gene is part of a targeted genomic complex (e.g., a gene that has at least part of its genomic sequence as part of a target genomic complex, e.g., inside an anchor sequence-mediated conjunction), which genomic complex is targeted by one or more modulating agents as described herein. In some embodiments, a target gene is modulated by a genomic sequence of a target gene being directly contacted by a modulating agent as described herein. In some embodiments, a target gene is modulated by one or more components of a genomic complex of which it is part being contacted by a modulating agent as describe herein. In some embodiments, a target gene is outside of a target genomic complex, for example, is a gene that encodes a component of a target genomic complex (e.g., a subunit of a transcription factor). In some embodiments, a target gene is associated with a genomic complex as described herein.
Targeting moiety: As used herein, the term “targeting moiety” means an agent or entity that specifically targets, e.g., binds, a genomic sequence element (e.g., an expression control sequence or anchor sequence) proximal to and/or operably linked to a target gene (e.g., FXN).
Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) and/or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

The patent or application file contains at least one drawing executed in color. Copies of this patent or patent application publication with color drawing(s) will be provided by the Office upon request and payment of the necessary fee.

### BRIEF DESCRIPTION OF THE FIGURES

FIG. 1A shows a Western blot against FXN for control primary fibroblasts (HDFn-normal) and for FRDA patient derived fibroblasts (GM04078). FIG. 1B shows a graph of FXN protein expression as measured by ELISA in HDFn and GM04078 cells. FIG. 1C shows a graph of aconitase activity in HDFn and GM04078 cells.

FIG. 2A shows a graph of FXN gene expression relative to HPRT1 reference gene in untreated GM04078 cells (Ctrl), GM04078 treated with modulating agent with non-targeting sgRNA (NT) or GM04078 cells treated with modulating agent with one of several pools of sgRNAs targeting FXN gene for 48 hours. FIG. 2B shows a graph of FXN gene expression relative to GAPDH reference gene in untreated GM04078 cells (Ctrl), GM04078 treated with modulating agent with non-targeting sgRNA (NT) or GM04078 cells treated with modulating agent with one of several pools of sgRNAs targeting FXN gene for 48 hours

FIG. 3A shows a graph of FXN gene expression relative to HPRT1 in GM04078 cells treated with modulating agent with a non-targeting sgRNA (NT), or GM04078 cells treated with modulating agent with one of several pools of sgRNAs 48 hours post LNP transfection or 72 hours post transfection. FIG. 3B shows a Western blot against FXN showing FXN levels untreated GM04078 cells (Ctrl), GM04078 treated with modulating agent with non-targeting sgRNA (NT), or GM04078 cells treated with modulating agent with one of several pools of sgRNAs 48 hours post LNP transfection or 72 hours post transfection.

FIG. 4A shows a graph of FXN gene expression relative to HPRT1 reference gene in GM04078 and HDFn cell lines following treatment with modulating agents at 48 hr post LNP transfection. FIG. 4B shows a graph of FXN gene expression relative to GAPDH reference gene in GM04078 and HDFn cell lines following treatment with modulating agents at 48 hr post LNP transfection. Ctrl: untreated (Ctrl); NT: treated with modulating agent with non-targeting sgRNA; Pool1 or Pool4: treated with modulating agent with either pool 1 or pool 4 of sgRNAs.

FIG. 5 shows a graph of aconitase activity as a rate (nmol/min/mg) for untreated GM04078 control cells, GM04078 cells treated with modulating agent with non-targeting sgRNA (NTC), and GM04078 cells treated with modulating agent with one of several pools of sgRNAs (Pool 1 or Pool 4).

FIG. 6 shows a diagram of the FXN gene, the repeat region, and the position of sgRNAs included in the sgRNA pools.

FIG. 7 shows a timeline of seeding and processing of cells for RNA analysis (top), and a diagram of the regions of genomic DNA targeted by pools of exemplary sgRNAs (bottom).

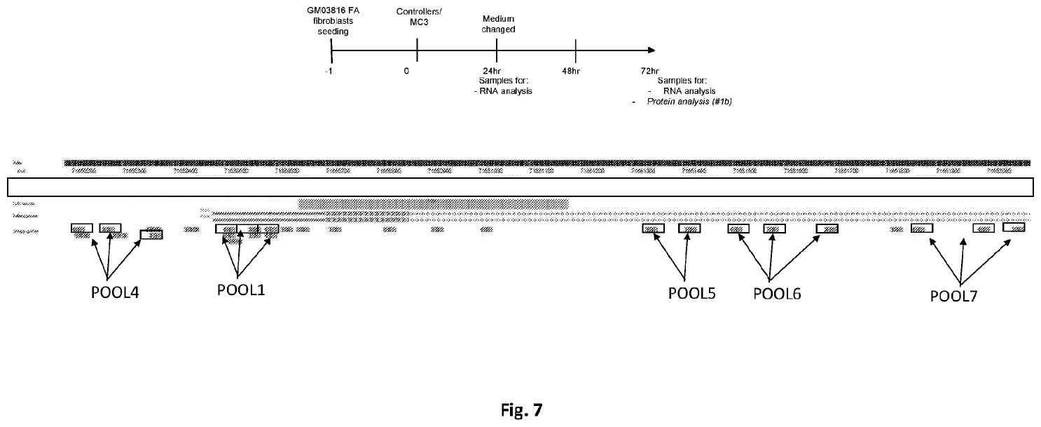

FIG. 8 shows graphs of FXN expression in WT iPSC-derived Cardiomyocytes (iCardiomyocytes) and WT iPSC-derived Glutamatergic Cortical Neurons (iNeurons) after treatment with exemplary fusion molecules (dCas9-VPR at left and dCas9-p300 at right).

FIG. 9 shows a graph of relative FXN expression as measured by RNA level in FRDA patient-derived fibroblasts treated with modulating agents comprising targeting moieties comprising TAL effector molecules or dCas9, and effector moieties comprising VPR.

FIG. 10 shows a graph of relative FXN expression as measured by protein level in FRDA patient-derived fibroblasts treated with modulating agents comprising targeting moieties comprising TAL effector molecules or dCas9, and effector moieties comprising VPR.

FIG. 11 shows graphs of relative FXN expression as measured by RNA level in mice injected with a modulating agent comprising a fusion molecule comprising dCas9-VPR at increasing amounts of time post-injection.

FIG. 12 shows graphs of relative FXN expression as measured by protein level in mice injected with a modulating agent comprising a fusion molecule comprising dCas9-VPR at increasing amounts of time post-injection.

FIG. 13 shows graphs of relative FXN expression as measured by RNA level in cells derived from FRDA patients or normal patients, wherein cells were treated with a modulating agent comprising a fusion molecule comprising dCas9-VPR at various times after treatment.

FIG. 14 shows graphs of relative FXN expression as measured by RNA level in cells derived from FRDA patients or normal patients, wherein cells were treated with a modulating agent comprising a fusion molecule comprising dCas9-p300 at various times after treatment.

FIG. 15 shows graphs of FXN expression as measured by protein level in FRDA and normal patient derived iPSC cardiomyocytes at increasing amounts of time post-treatment with a modulating agent comprising a fusion molecule comprising either dCas9-VPR or dCas9-p300.

### DETAILED DESCRIPTION

Provided herein are compositions and methods for modulating, e.g., increasing, frataxin (FXN) expression, e.g., in a subject in need thereof. FRDA is associated with an autosomal GAA repeat expansion in the FXN gene which reduces the level of FXN protein expression. Without wishing to be bound by theory, it is thought that increasing the levels of FXN protein in a subject (e.g., overall, or in a specific target tissue or tissues) suffering from FRDA may lessen or eliminate the symptoms of FRDA. The present disclosure provides, in part, modulating agents comprising a targeting moiety that binds to a genomic sequence element (e.g., an expression control element) operably linked to a target gene (e.g., FXN) and an effector moiety capable of modulating expression of the target gene when localized by the targeting moiety. In some embodiments, the modulating agents disclosed herein specifically bind to an expression control element (e.g., a promoter or enhancer) operably linked to the FXN gene via the targeting moiety and the effector moiety modulates expression of FXN.

The disclosure further provides nucleic acids encoding said modulating agents and compositions and methods for delivering said nucleic acids. Further provided are methods for increasing FXN expression in a cell using the modulating agents described herein.

### Modulating Agents

As described herein, the present disclosure provides technologies for modulating (e.g., increasing) expression of a target gene, e.g., FXN, by contacting a cell with a modulating agent as described herein. In some embodiments, a modulating agent comprises a targeting moiety and an effector moiety. In some embodiments, a modulating agent comprises a targeting moiety and one effector moiety. In some embodiments, a modulating agent comprises a targeting moiety and a plurality of effector moieties (e.g., 1, 2, 3, 4, 5, 6, 7, 8, 9, 10, 11, 12, 13, 14, 15, 16, 17, 18, 19, 20, or more effector domains (and optionally, less than 20, 19, 18, 17, 16, 15, 14, 13, 12, 11, 10, 9, 8, 7, 6, 5, 4, 3, or 2 effector domains)).

In general, a modulating agent as described herein binds (e.g., via a targeting moiety) a genomic sequence element proximal to and/or operably linked to a target gene (e.g., FXN). In some embodiments, binding of the modulating agent to the genomic sequence element modulates (e.g., increases) expression of the target gene (e.g., FXN). For example, binding of a modulating agent comprising an effector moiety that recruits or inhibits recruitment of components of the transcription machinery to the genomic sequence element may modulate (e.g., increase) expression of the target gene (e.g., FXN). As a further example, binding of a modulating agent comprising an effector moiety with an enzymatic activity (e.g., an epigenetic modifying moiety) may modulate (e.g., increase) expression of the target gene (e.g., FXN) through the localized enzymatic activity of the effector moiety. As a further example, both binding of a modulating agent to a genomic sequence element and the localized enzymatic activity of a modulating agent may contribute to the resulting modulation (e.g., increase) in expression of the target gene (e.g., FXN).

In some embodiments, a modulating agent increases expression of a target gene (e.g., FXN) by promoting transcription of the target gene. A modulating agent may recruit a component of the transcription machinery to the target gene or an expression control sequence operably linked to the target gene. A modulating agent may inhibit interaction of an inhibitor of transcription with the target gene or an expression control sequence operably linked to the target gene.

In some embodiments, increasing expression comprises increasing the level of mRNA encoded by the target gene (e.g., FXN). In some embodiments, increasing expression comprises increasing the level of protein encoded by the target gene (e.g., FXN). In some embodiments, increasing expression comprises both increasing the level of mRNA and protein encoded by the target gene. In some embodiments, the expression of a target gene (e.g., FXN) in a cell contacted by or comprising the modulating agent is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× higher than the level of expression of the target gene in a similar cell not contacted by or comprising the modulating agent. Expression of a target gene may be assayed by methods known to those of skill in the art, including RT-PCR, ELISA, or Western blot. Expression level of FXN in a subject, e.g., a patient, e.g., a patient who has FRDA, may be assessed by evaluating blood (e.g., whole blood) levels of FXN, e.g., by the method of either Oglesbee et al. Clin Chem. 2013 October; 59(10):1461-9. doi: 10.1373/clinchem.2013.207472 or Deutsch et al. J Neurol Neurosurg Psychiatry. 2014 September; 85(9):994-1002. doi: 10.1136/jnnp-2013-306788, the contents of which are hereby incorporated by reference in their entirety.

A modulating agent of the present disclosure can be used to increase expression of a target gene (e.g., FXN) in a cell for a time period. In some embodiments, the expression of a target gene in a cell contacted by or comprising the modulating agent is appreciably increased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a target gene in a cell contacted by or comprising the modulating agent is appreciably increased for no more than 10, 9, 8, 7, 6, 5, 4, 3, 2, or 1 years.

A modulating agent may comprise a plurality of effector moieties, where each effector moiety comprises a different functionality than the other effector moieties. For example, a modulating agent may comprise two effector moieties, where the first effector moiety comprises transcriptional activator functionality and the second effector moiety comprises a DNA demethylase functionality. In some embodiments, a modulating agent comprises effector moieties whose functionalities are complementary to one another with regard to increasing expression of a target gene (e.g., FXN), e.g., where the functionalities together enable promotion of expression and, optionally, do not promote or negligibly promote expression when present individually. In some embodiments, a modulating agent comprises a plurality of effector moieties, wherein each effector moiety complements each other effector moiety, e.g., each effector moiety increases expression of a target gene (e.g., FXN).

In some embodiments, a modulating agent comprises a combination of effector moieties whose functionalities synergize with one another with regard to increasing expression of a target gene (e.g., FXN). Without wishing to be bound by theory, it is thought that epigenetic modifications to a genomic locus are cumulative, in that multiple transcription activating epigenetic markers (e.g., multiple different types of epigenetic markers and/or more extensive marking of a given type) individually together promote expression more effectively than individual modifications alone (e.g., producing a greater increase in expression and/or a longer-lasting increase in expression). In some embodiments, a modulating agent comprises a plurality of effector moieties, wherein each effector moiety synergizes with each other effector moiety, e.g., each effector moiety increases expression of a target gene (e.g., FXN). In some embodiments, a modulating agent (comprising a plurality of effector moieties which synergize with one another) is more effective at promoting expression of a target gene (e.g., FXN) than a modulating agent comprising an individual effector moiety. In some embodiments, a modulating agent comprising said plurality of effector moieties is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at increasing expression of a target gene (e.g., FXN) than a modulating agent comprising an individual effector moiety.

In some embodiments, a modulating agent modulates (e.g., increases) expression of a target gene (e.g., FXN) by altering one or more epigenetic markers associated with the target gene or an expression control sequence operably linked thereto. In some embodiments, altering comprises increasing the level of an epigenetic marker associated with the target gene or an expression control sequence operably linked thereto. In some embodiments, altering comprises decreasing the level of an epigenetic marker associated with the target gene or an expression control sequence operably linked thereto. Epigenetic markers include, but are not limited to, DNA methylation, histone methylation, and histone deacetylation.

In some embodiments, altering the level of an epigenetic marker increases the level of the epigenetic marker associated with the target gene or an expression control sequence operably linked thereto by at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× higher than the level of the epigenetic marker associated with the target gene or an expression control sequence operably linked thereto in a cell not contacted by or comprising the modulating agent. In some embodiments, altering the level of an epigenetic marker decreases the level of the epigenetic marker associated with the target gene or an expression control sequence operably linked thereto by at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of the epigenetic marker associated with the target gene or an expression control sequence operably linked thereto in a cell not contacted by or comprising the modulating agent. The level of an epigenetic marker may be assayed by methods known to those of skill in the art, including whole genome bisulfite sequencing, reduced representation bisulfite sequencing, bisulfite amplicon sequencing, methylation arrays, pyrosequencing, ChIP-seq, or ChIP-qPCR.

A modulating agent of the present disclosure can be used to alter the level of an epigenetic marker associated with the target gene or an expression control sequence operably linked thereto in a cell for a time period. In some embodiments, the level of the epigenetic marker associated with the target gene or an expression control sequence operably linked thereto in a cell contacted by or comprising the modulating agent is appreciably increased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). In some embodiments, the level of the epigenetic marker associated with the target gene or an expression control sequence operably linked thereto in a cell contacted by or comprising the modulating agent is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the level of an epigenetic marker associated with the target gene or an expression control sequence operably linked thereto in a cell contacted by or comprising the modulating agent is appreciably increased or decreased for no more than 10, 9, 8, 7, 6, 5, 4, 3, 2, or 1 years.

A modulating agent may be or comprise a fusion molecule. In some embodiments, a fusion molecule comprises a targeting moiety and an effector moiety which are covalently connected to one another, e.g., by a peptide bond.

In some embodiments, a modulating agent, e.g., the targeting moiety of a fusion molecule, comprises no more than 100, 90, 80, 70, 60, 50, 40, 30, or 20 nucleotides (and optionally at least 10, 20, 30, 40, 50, 60, 70, 80, or 90 nucleotides). In some embodiments, a modulating agent, e.g., the effector moiety of a fusion molecule, comprises no more than 2000, 1900, 1800, 1700, 1600, 1500, 1400, 1300, 1200, 1100, 1000, 900, 800, 700, 600, 500, 400, 300, 200, or 100 amino acids (and optionally at least 50, 100, 200, 300, 400, 500, 600, 700, 800, 900, 1000, 1100, 1200, 1300, 1400, 1500, 1600, 1700, 1800, or 1900 amino acids). In some embodiments, a modulating agent, e.g., the effector moiety of a fusion molecule, comprises 100-2000, 100-1900, 100-1800, 100-1700, 100-1600, 100-1500, 100-1400, 100-1300, 100-1200, 100-1100, 100-1000, 100-900, 100-800, 100-700, 100-600, 100-500, 100-400, 100-300, 100-200, 200-2000, 200-1900, 200-1800, 200-1700, 200-1600, 200-1500, 200-1400, 200-1300, 200-1200, 200-1100, 200-1000, 200-900, 200-800, 200-700, 200-600, 200-500, 200-400, 200-300, 300-2000, 300-1900, 300-1800, 300-1700, 300-1600, 300-1500, 300-1400, 300-1300, 300-1200, 300-1100, 300-1000, 300-900, 300-800, 300-700, 300-600, 300-500, 300-400, 400-2000, 400-1900, 400-1800, 400-1700, 400-1600, 400-1500, 400-1400, 400-1300, 400-1200, 400-1100, 400-1000, 400-900, 400-800, 400-700, 400-600, 400-500, 500-2000, 500-1900, 500-1800, 500-1700, 500-1600, 500-1500, 500-1400, 500-1300, 500-1200, 500-1100, 500-1000, 500-900, 500-800, 500-700, 500-600, 600-2000, 600-1900, 600-1800, 600-1700, 600-1600, 600-1500, 600-1400, 600-1300, 600-1200, 600-1100, 600-1000, 600-900, 600-800, 600-700, 700-2000, 700-1900, 700-1800, 700-1700, 700-1600, 700-1500, 700-1400, 700-1300, 700-1200, 700-1100, 700-1000, 700-900, 700-800, 800-2000, 800-1900, 800-1800, 800-1700, 800-1600, 800-1500, 800-1400, 800-1300, 800-1200, 800-1100, 800-1000, 800-900, 900-2000, 900-1900, 900-1800, 900-1700, 900-1600, 900-1500, 900-1400, 900-1300, 900-1200, 900-1100, 900-1000, 1000-2000, 1000-1900, 1000-1800, 1000-1700, 1000-1600, 1000-1500, 1000-1400, 1000-1300, 1000-1200, 1000-1100, 1100-2000, 1100-1900, 1100-1800, 1100-1700, 1100-1600, 1100-1500, 1100-1400, 1100-1300, 1100-1200, 1200-2000, 1200-1900, 1200-1800, 1200-1700, 1200-1600, 1200-1500, 1200-1400, 1200-1300, 1300-2000, 1300-1900, 1300-1800, 1300-1700, 1300-1600, 1300-1500, 1300-1400, 1400-2000, 1400-1900, 1400-1800, 1400-1700, 1400-1600, 1400-1500, 1500-2000, 1500-1900, 1500-1800, 1500-1700, 1500-1600, 1600-2000, 1600-1900, 1600-1800, 1600-1700, 1700-2000, 1700-1900, 1700-1800, 1800-2000, 1800-1900, or 1900-2000 amino acids.

A modulating agent may comprise nucleic acid, e.g., one or more nucleic acids. The term “nucleic acid” refers to any compound that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is or comprises more than 50% ribonucleotides and is referred to herein as a ribonucleic acid (RNA). In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. As used herein, “recombinant” when used to describe a nucleic acid refers to any nucleic acid that does not naturally occur. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, nucleic acids may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

In some embodiments, a targeting moiety comprises or is nucleic acid. In some embodiments, an effector moiety comprises or is nucleic acid. In some embodiments, a nucleic acid that may be included in a moiety may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. A nucleic acid sequence suitable for use in a modulating agent may include modified oligonucleotides (e.g., chemical modifications, such as modifications that alter backbone linkages, sugar molecules, and/or nucleic acid bases) and/or artificial nucleic acids. In some embodiments, a nucleic acid sequence includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules. In some embodiments, a nucleic acid may include one or more residues that is not a naturally-occurring DNA or RNA residue, may include one or more linkages that is/are not phosphodiester bonds (e.g., that may be, for example, phosphorothioate bonds, etc), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

Some examples of nucleic acids include, but are not limited to, a nucleic acid that hybridizes to an endogenous target gene, e.g., FXN, (e.g., gRNA or antisense ssDNA as described herein elsewhere), a nucleic acid that hybridizes to an exogenous nucleic acid such as a viral DNA or RNA, nucleic acid that hybridizes to an RNA, a nucleic acid that interferes with gene transcription, a nucleic acid that interferes with RNA translation, a nucleic acid that stabilizes RNA or destabilizes RNA such as through targeting for degradation, a nucleic acid that interferes with a DNA or RNA binding factor through interference of its expression or its function, a nucleic acid that is linked to a intracellular protein or protein complex and modulates its function, etc.

In some embodiments, a modulating agent comprises one or more nucleoside analogs. In some embodiments, a nucleic acid sequence may include in addition or as an alternative to one or more natural nucleosides nucleosides, e.g., purines or pyrimidines, e.g., adenine, cytosine, guanine, thymine and uracil, one or more nucleoside analogs. In some embodiments, a nucleic acid sequence includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

Targeting Moiety

A targeting moiety refers to an agent or entity that specifically targets, e.g., binds, a genomic sequence element (e.g., an expression control sequence or anchor sequence) proximal to and/or operably linked to a target gene (e.g., FXN). In some embodiments, a targeting moiety targets, e.g., binds, a component of a genomic complex (e.g., ASMC). In some embodiments, a targeting moiety targets, e.g., binds, an expression control sequence (e.g., a promoter or enhancer) operably linked to FXN. In some embodiments, a targeting moiety targets, e.g., binds, a target gene (e.g., FXN) or a part of a target gene. The target of a targeting moiety may be referred to as its targeted component. A targeted component may be any genomic sequence element operably linked to a target gene, or the target gene itself, including but not limited to a promoter, enhancer, anchor sequence, exon, intron, UTR encoding sequence, a splice site, or a transcription start site.

In some embodiments, interaction between a targeting moiety and its targeted component interferes with one or more other interactions that the targeted component would otherwise make. In some embodiments, binding of a targeting moiety to a targeted component prevents the targeted component from interacting with another transcription factor, genomic complex component, or genomic sequence element. In some embodiments, binding of a targeting moiety to a targeted component decreases binding affinity of the targeted component for another transcription factor, genomic complex component, or genomic sequence element. In some embodiments, K_{D} of a targeted component for another transcription factor, genomic complex component, or genomic sequence element increases by at least 1.05× (i.e., 1.05 times), 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20×, 50×, or 100× (and optionally no more than 20×, 10×, 9×, 8×, 7×, 6×, 5×, 4×, 3×, 2×, 1.9×, 1.8×, 1.7×, 1.6×, 1.5×, 1.4×, 1.3×, 1.2×, or 1.1×) in presence of a modulating agent comprising the targeting moiety than in the absence of the modulating agent comprising the targeting moiety. Changes in K_{D} of a targeted component for another transcription factor, genomic complex component, or genomic sequence element may be evaluated, for example, using ChIP-Seq or ChIP-qPCR.

In some embodiments, binding of a targeting moiety to a targeted component alters, e.g., decreases, the level of a genomic complex (e.g., ASMC) comprising the targeted component. In some embodiments, the level of a genomic complex (e.g., ASMC) comprising the targeted component decreases by at least 10, 20, 30, 40, 50, 60, 70, 80, 90, or 100% (and optionally, up to 100, 90, 80, 70, 60, 50, 40, 30, or 20%) in the presence of a modulating agent comprising the targeting moiety relative to the absence of said modulating agent. In some embodiments, binding of a targeting moiety to a targeted component alters, e.g., decreases, occupancy of the genomic complex (e.g., ASMC) at a genomic sequence element (e.g., a target gene, or a expression control sequence operably linked thereto). In some embodiments, occupancy decreases by at least 10, 20, 30, 40, 50, 60, 70, 80, 90, or 100% (and optionally, up to 100, 90, 80, 70, 60, 50, 40, 30, or 20%) in the presence of a modulating agent comprising the targeting moiety relative to the absence of said modulating agent. Changes in genomic complex formation, affinity of targeted components for other complex components, and/or changes in topology of genomic DNA impacted by a genomic complex may be evaluated, for example, using HiChIP, ChIAPET, 4C, or 3C, e.g., HiChIP.

In some embodiments, binding of a targeting moiety to a targeted component alters, e.g., decreases, the occupancy of the genomic complex (e.g., ASMC) at a genomic sequence element (e.g., a gene, promoter, or enhancer, e.g., associated with the genomic or transcription complex). In some embodiments, binding of a targeting moiety to a targeted component decreases occupancy of the genomic complex (e.g., ASMC) at a genomic sequence element by at least 10, 20, 30, 40, 50, 60, 70, 80, 90, or 100% (and optionally, up to 100, 90, 80, 70, 60, 50, 40, 30, or 20%) in the presence of a modulating agent comprising the targeting moiety relative to the absence of said modulating agent. In some embodiments, occupancy refers to the frequency with which an element can be found associated with another element, e.g., as determined by HiC, ChIP, immunoprecipitation, or other association measuring assays known in the art. In some embodiments, occupancy can be determined using integrity index (e.g., a change in integrity index may correspond to a change in occupancy).

In some embodiments, binding of a targeting moiety to a targeted component alters, e.g., decreases the occupancy of the targeted component in/at the genomic complex (e.g., ASMC). In some embodiments, binding of a targeting moiety to a targeted component decreases occupancy of the targeted component in/at the genomic complex (e.g., ASMC) by at least 10, 20, 30, 40, 50, 60, 70, 80, 90, or 100% (and optionally, up to 100, 90, 80, 70, 60, 50, 40, 30, or 20%) in the presence of a modulating agent comprising the targeting moiety relative to the absence of said modulating agent.

In some embodiments, binding of a targeting moiety to a targeted component alters, e.g., increases, the expression of a target gene (e.g., FXN) associated with and/or operably linked to the targeted component. In some embodiments, binding of a targeting moiety to a targeted component alters, e.g., increases, the expression of a target gene (e.g., FXN) associated with the genomic complex (e.g., ASMC) comprising the targeted component. In some embodiments, the expression of the target gene increases by at least 10, 20, 30, 40, 50, 60, 70, 80, 90, 100, 200, 300, 400, 500, 600, 700, 800, 900, or 1000% (and optionally, up to 1000, 900, 800, 700, 600, 500, 400, 300, or 200%) in the presence of a modulating agent comprising the targeting moiety relative to the absence of said modulating agent.

In some embodiments, a targeting moiety is designed and/or administered so that it specifically targets, e.g., binds, a particular genomic sequence element (e.g., a specific genomic complex (e.g., ASMC) comprising said genomic sequence element) relative to other genomic sequence elements that may be present in the same system (e.g., cell, tissue, etc.). In some embodiments, a targeting moiety comprises a nucleic acid sequence complementary to a targeted component, e.g., an expression control sequence, anchor sequence, or target gene (e.g., FXN). In some embodiments, a targeting moiety comprises a nucleic acid sequence that is at least 10%, 15%, 20%, 25%, 30%, 35%, 40%, 45%, 50%, 55%, 60%, 65%, 70%, 75%, 80%, 85%, 90%, 95%, 99%, or 100% complementary to a targeted component.

In some embodiments, a targeting moiety may be or comprise a CRISPR/Cas molecule, a TAL effector molecule, a Zn finger molecule, or a nucleic acid molecule.

### CRISPR/Cas Molecules

In some embodiments, a targeting moiety is or comprises a CRISPR/Cas molecule. A CRISPR/Cas molecule comprises a protein involved in the clustered regulatory interspaced short palindromic repeat (CRISPR) system, e.g., a Cas protein, and optionally a guide RNA, e.g., single guide RNA (sgRNA).

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 1. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 1

| | | | | | Mutations to | |
|---|---|---|---|---|---|---|
| | | | # of | | alter PAM | Mutations to make |
| Name | Enzyme | Species | AAs | PAM | recognition | catalytically dead |
| FnCas9 | Cas9 | Francisella | 1629 | 5′-NGG-3′ | Wt | D11A/H969A/N995A |
| | | novicida | | | | |
| FnCas9 | Cas9 | Francisella | 1629 | 5′-YG-3′ | E1369R/E1449H/ | D11A/H969A/N995A |
| RHA | | novicida | | | R1556A | |
| SaCas9 | Cas9 | Staphylococcus | 1053 | 5′-NNGRRT-3′ | Wt | D10A/H557A |
| | | aureus | | | | |
| SaCas9 | Cas9 | Staphylococcus | 1053 | 5′-NNNRRT-3′ | E782K/N968K/R1015H | D10A/H557A |
| KKH | | aureus | | | | |
| SpCas9 | Cas9 | Streptococcus | 1368 | 5′-NGG-3′ | Wt | D10A/D839A/H840A/ |
| | | pyogenes | | | | N863A |
| SpCas9 | Cas9 | Streptococcus | 1368 | 5′-NGA-3′ | D1135V/R1335Q/ | D10A/D839A/H840A/ |
| VQR | | pyogenes | | | T1337R | N863A |
| AsCpf1 | Cpf1 | Acidaminococcus | 1307 | 5′-TYCV-3′ | S542R/K607R | E993A |
| RR | | sp. BV3L6 | | | | |
| AsCpf1 | Cpf1 | Acidaminococcus | 1307 | 5′-TATV-3′ | S542R/K548V/N552R | E993A |
| RVR | | sp. BV3L6 | | | | |
| FnCpf1 | Cpf1 | Francisella | 1300 | 5′-NTTN-3′ | Wt | D917A/E1006A/D1255A |
| | | novicida | | | | |
| NmCas9 | Cas9 | Neisseria | 1082 | 5′-NNNGATT-3′ | Wt | D16A/D587A/H588A/ |
| | | meningitidis | | | | N611A |

In some embodiments, the Cas protein is modified to deactivate the nuclease, e.g., nuclease-deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas9 binding to a DNA sequence may interfere with transcription at that site by steric hindrance. In some embodiments, a targeting moiety is or comprises a catalytically inactive Cas9, e.g., dCas9. Many catalytically inactive Cas9 proteins are known in the art. In some embodiments, dCas9 comprises mutations in each endonuclease domain of the Cas protein, e.g., D10A and H840A mutations.

In some embodiments, a targeting moiety may comprise a Cas molecule comprising or linked (e.g., covalently) to a gRNA. A gRNA is a short synthetic RNA composed of a “scaffold” sequence necessary for Cas-protein binding and a user-defined ˜20 nucleotide targeting sequence for a genomic target. In practice, guide RNA sequences are generally designed to have a length of between 17-24 nucleotides (e.g., 19, 20, or 21 nucleotides) and be complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol., 985-991.

In some embodiments, a gRNA comprises a nucleic acid sequence that is complementary to a DNA sequence associated with a target gene. In some embodiments, the DNA sequence is, comprises, or overlaps an expression control element that is operably linked to the target gene. In some embodiments, the DNA sequence is, comprises, or overlaps a genomic sequence recited in Table 3. In some embodiments, a gRNA comprises a nucleic acid sequence that is at least 80, 85, 90, 95, 99, or 100% complementary to a genomic sequence recited in Table 3. In some embodiments, the gRNA comprises a nucleic acid sequence selected from SEQ ID NOs: 4-26 or a sequence that has at least 80, 85, 90, 95, or 99% identity to a sequence selected from SEQ ID NOs: 4-26. In some embodiments, a gRNA for use with a targeting moiety that comprises a Cas molecule is an sgRNA.

### TAL Effector Molecules

In some embodiments, a targeting moiety is or comprises a TAL effector molecule. A TAL effector molecule, e.g., a TAL effector molecule that specifically binds a DNA sequence, comprises a plurality of TAL effector domains or fragments thereof, and optionally one or more additional portions of naturally occurring TAL effectors (e.g., N- and/or C-terminal of the plurality of TAL effector domains).

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector feature a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 2 listing exemplary repeat variable diresidues (RVD) and their correspondence to nucleic acid base targets.

TABLE 2
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of the TAL effector molecule of the present invention may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicolastrain BLS256 (Bogdanove et al. 2011). As used herein, the TAL effector domain in accordance with the present invention comprises an RVD domain as well as flanking sequence(s) (sequences on the N-terminal and/or C-terminal side of the RVD) also from the naturally occurring TAL effector. It may comprise more or fewer repeats than the RVD of the naturally occurring TAL effector. The TAL effector molecule of the present invention is designed to target a given DNA sequence based on the above code and others known in the art. The number of TAL effector domains (e.g., repeats (monomers or modules)) and their specific sequence are selected based on the desired DNA target sequence. For example, TAL effector domains, e.g., repeats, may be removed or added in order to suit a specific target sequence. In an embodiment, the TAL effector molecule of the present invention comprises between 6.5 and 33.5 TAL effector domains, e.g., repeats. In an embodiment, TAL effector molecule of the present invention comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the expression repression system, e.g., the expression repressor comprising the TAL effector molecule. In general, TALE binding is inversely correlated with the number of mismatches. In some embodiments, the TAL effector molecule of a expression repressor of the present invention comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the expression repression system, e.g., the expression repressor comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present invention may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule of an expression repressor of the present invention. Accordingly, in an embodiment, a TAL effector molecule of the present invention comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

### Zn Finger Molecules

In some embodiments, a targeting moiety is or comprises a Zn finger molecule. A Zn finger molecule comprises a Zn finger protein, e.g., a naturally occurring Zn finger protein or engineered Zn finger protein, or fragment thereof.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the DNA-targeting moiety comprises a Zn finger molecule comprising an engineered zinc finger protein that binds (in a sequence-specific manner) to a target DNA sequence. In some embodiments, the Zn finger molecule comprises one Zn finger protein or fragment thereof. In other embodiments, the Zn finger molecule comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger molecule comprises at least three Zn finger proteins. In some embodiments, the Zn finger molecule comprises four, five or six fingers. In some embodiments, the Zn finger molecule comprises 8, 9, 10, 11 or 12 fingers. In some embodiments, a Zn finger molecule comprising three Zn finger proteins recognizes a target DNA sequence comprising 9 or 10 nucleotides. In some embodiments, a Zn finger molecule comprising four Zn finger proteins recognizes a target DNA sequence comprising 12 to 14 nucleotides. In some embodiments, a Zn finger molecule comprising six Zn finger proteins recognizes a target DNA sequence comprising 18 to 21 nucleotides.

In some embodiments, a Zn finger molecule comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

In some embodiments, a targeting moiety is or comprises a DNA-binding domain from a nuclease. For example, the recognition sequences of homing endonucleases and meganucleases such as I-SceI, I-CeuI, PI-Pspl, PI-Sce, I-SceIV, I-Csml, I-PanI, I-SceII, I-Ppol, I-SceIII, I-CreI, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

### Target Sequences

A targeting moiety targets, e.g., binds, a genomic sequence element proximal to and/or operably linked to a target gene (e.g., FXN). In some embodiments, the genomic sequence element is or comprises an expression control sequence. In some embodiments, the genomic sequence element is or comprises an anchor sequence. In some embodiments, the genomic sequence element is or comprises the target gene (e.g., FXN) or a part of the target gene. In some embodiments, a targeting moiety binds to a target sequence comprised by or partially comprised by a genomic sequence element. In some embodiments, a targeting moiety binds to a target sequence that is at least 10, 11, 12, 13, 14, 15, 16, 17, 18, 19, 20, 21, 22, 23, 24, 25, 26, 27, 28, 29, 30, 31, 32, 33, 34, or 35 bases long (and optionally no more than 40, 39, 38, 37, 36, 35, 34, 33, 32, 31, 30, 29, 28, 27, 26, 25, 24, 23, 22, 21, or 20 bases long). In some embodiments, a targeting moiety binds to a target sequence that is 10-30, 15-30, 15-25, 18-24, 19-23, 20-23, 21-23, or 22-23 bases long. In some embodiments, the target sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases long. Anchor Sequences

In general, an anchor sequence is a genomic sequence element to which a genomic complex component, e.g., nucleating polypeptide, binds specifically. In some embodiments, binding to an anchor sequence nucleates genomic complex (e.g., ASMC) formation.

An anchor sequence-mediated conjunction (ASMC) comprises a plurality of anchor sequences, e.g., two or more anchor sequences. In some embodiments, anchor sequences can be manipulated or altered to modulate (e.g., disrupt) a naturally occurring genomic complex (e.g., ASMC) or to form a new genomic complex (e.g., ASMC) (e.g., to form a non-naturally occurring genomic complex (e.g., ASMC) with an exogenous or altered anchor sequence). Such alterations may modulate gene expression by, e.g., changing topological structure of DNA, e.g., thereby modulating the ability of a target gene to interact with gene regulation and control factors (e.g., a expression control sequence, e.g., promoter, enhancer, or repressor sequence).

In some embodiments, chromatin structure is modified by substituting, adding or deleting one or more nucleotides within an anchor sequence. In some embodiments, chromatin structure is modified by substituting, adding, or deleting one or more nucleotides within an anchor sequence of an anchor sequence-mediated conjunction.

In some embodiments, an anchor sequence comprises a nucleating polypeptide binding motif, e.g., a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO:1), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2).

In some embodiments, an anchor sequence comprises SEQ ID NO:1 or SEQ ID NO:2 or a sequence at least 75%, at least 80%, at least 90%, at least 95%, at least 96%, at least 97%, at least 98%, at least 99% identical to either SEQ ID NO:1 or SEQ ID NO:2.

In some embodiments, an anchor sequence-mediated conjunction comprises at least a first anchor sequence and a second anchor sequence. For example, in some embodiments, a first anchor sequence and a second anchor sequence may each comprise a nucleating polypeptide binding motif, e.g., each comprises a CTCF binding motif.

In some embodiments, a first anchor sequence and second anchor sequence comprise different sequences, e.g., a first anchor sequence comprises a CTCF binding motif and a second anchor sequence comprises an anchor sequence other than a CTCF binding motif. In some embodiments, each anchor sequence comprises a nucleating polypeptide binding motif and one or more flanking nucleotides on one or both sides of a nucleating polypeptide binding motif.

Two CTCF-binding motifs (e.g., contiguous or non-contiguous CTCF binding motifs) that can form an ASMC may be present in a genome in any orientation, e.g., in the same orientation (tandem) either 5′-3′ (left tandem, e.g., the two CTCF-binding motifs that comprise SEQ ID NO:1) or 3′-5′ (right tandem, e.g., the two CTCF-binding motifs comprise SEQ ID NO:2), or convergent orientation, where one CTCF-binding motif comprises SEQ ID NO:1 and another other comprises SEQ ID NO:2. CTCFBSDB 2.0: Database For CTCF binding motifs And Genome Organization (http://insulatordb.uthsc.edu/) can be used to identify CTCF binding motifs associated with a target gene.

In some embodiments, an anchor sequence comprises a CTCF binding motif associated with a target gene (e.g., FXN), wherein the target gene is associated with a disease, disorder and/or condition (e.g., FRDA).

In some embodiments, methods of the present disclosure comprise modulating, e.g., disrupting, a genomic complex (e.g., ASMC), e.g., by modifying chromatin structure, by substituting, adding, or deleting one or more nucleotides within an anchor sequence, e.g., a nucleating polypeptide binding motif. One or more nucleotides may be specifically targeted, e.g., a targeted alteration, for substitution, addition or deletion within an anchor sequence, e.g., a nucleating polypeptide binding motif.

In some embodiments, a genomic complex (e.g., ASMC) may be altered by changing an orientation of at least one nucleating polypeptide binding motif. In some embodiments, an anchor sequence comprises a nucleating polypeptide binding motif, e.g., CTCF binding motif, and a targeting moiety introduces an alteration in at least one nucleating polypeptide binding motif, e.g., altering binding affinity for a nucleating polypeptide.

Expression Control Sequences

In some embodiments, a target gene (e.g., FXN) is associated with and/or operably linked with one or more expression control sequences. In some embodiments, a genomic complex (e.g., ASMC) colocalizes two or more genomic sequence elements that include one or more expression control sequences. Those skilled in the art are familiar with a variety of positive (e.g., promoters or enhancers) or negative (e.g., repressors or silencers) expression control sequences that are associated with genes. Typically, when a cognate regulatory protein is bound to such a expression control sequence, transcription from the associated gene(s) is altered (e.g., increased for a positive expression control sequence; decreased for a negative expression control sequence).

Promoter Sequences

In some embodiments, a target gene (e.g., FXN) is associated with and/or operably linked with a promoter. In some embodiments, a genomic complex (e.g., ASMC) colocalizes two or more genomic sequence elements, wherein the two or more genomic sequence elements include a promoter. Those skilled in the art are aware that a promoter is, typically, a sequence element that initiates transcription of an associated gene. Promoters are typically near the 5′ end of a gene, not far from its transcription start site.

As those of ordinary skill are aware, transcription of protein-coding genes in eukaryotic cells is typically initiated by binding of general transcription factors (e.g., TFIID, TFIIE, TFIIH, etc) and Mediator to core promoter sequences as a preinitiation complex that directs RNA polymerase II to the transcription start site, and in many instances remains bound to the core promoter sequences even after RNA polymerase escapes and elongation of the primary transcript is initiated.

In many embodiments, a promoter includes a sequence element, such as TATA, Inr, DPE, or BRE, but those skilled in the art are well aware that such sequences are not necessarily required to define a promoter. In some embodiments, a targeting moiety targets, e.g., binds, to a target sequence in a promoter operably linked to a target gene, where the target gene is FXN. In some embodiments, FXN is located on human chromosome 9. In some embodiments, the transcription start site (TSS) is the transcription start entry of the hg19 annotation of the human genome (GRCh37), retrieved via UCSC Table Browser (Karolchik D, Hinrichs A S, Furey T S, Roskin K M, Sugnet C W, Haussler D, Kent W J. The UCSC Table Browser data retrieval tool. Nucleic Acids Res. 2004 Jan. 1; 32 (Database issue):D493-6). In some embodiments, the TSS is at chromosome position 71650667 (e.g., in the Genome Reference Consortium Human Build 37 (GRCh37)). In some embodiments, an expression control sequence, e.g., promoter, operably linked to FXN comprises a sequence encompassing the approximately 1000 bases on either side of the TSS. In some embodiments, the target moiety binds to a target sequence comprising sequence positions that are at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 bases upstream from the TSS (and optionally no more than 500, 450, 400, 350, 300, 250, 200, 150, 100, 90, 80, 70, 60, 50, 40, 30, 20, or 10 bases upstream from the TSS). In some embodiments, the target moiety binds to a target sequence comprising sequence positions that are at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 bases downstream from the TSS (and optionally no more than 500, 450, 400, 350, 300, 250, 200, 150, 100, 90, 80, 70, 60, 50, 40, 30, 20, or 10 bases downstream from the TSS).

In some embodiments, the target moiety binds to a target sequence where the position nearest to the TSS is at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 bases upstream from the TSS (and optionally no more than 500, 450, 400, 350, 300, 250, 200, 150, 100, 90, 80, 70, 60, 50, 40, 30, 20, or 10 bases upstream from the TSS). In some embodiments, the target moiety binds to a target sequence where the position nearest to the TSS is at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 bases downstream from the TSS (and optionally no more than 500, 450, 400, 350, 300, 250, 200, 150, 100, 90, 80, 70, 60, 50, 40, 30, 20, or 10 bases downstream from the TSS).

In some embodiments, a targeting moiety targets, e.g., binds, to a target sequence where the position nearest to the TSS is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 bases upstream or downstream from the TSS. In some embodiments, a targeting moiety targets, e.g., binds, to a target sequence where the position nearest to the TSS is about 150 (e.g., 150) bases upstream. In some embodiments, a targeting moiety targets, e.g., binds, to a target sequence where the position nearest to the TSS is about 50 (e.g., 50) bases downstream.

In some embodiments, a targeting moiety binds to an exemplary target sequence chosen from Table 3 (e.g., specified by the Upstream and Downstream end columns of Table 3). In some embodiments, a targeting moiety comprises a nucleic acid sequence, e.g., an sgRNA, that is complementary or partially complementary (e.g., at all but 1, 2, 3, 4, 5, 6, 7, or 8 positions) to a target sequence (e.g., a target sequence of Table 3). Exemplary guide sequences (e.g., for use in an sgRNA of a targeting moiety) for binding exemplary target sequences are also provided in Table 3.

TABLE 3

| | | | Distance from | | |
|---|---|---|---|---|---|
| | | | TSS to nearert | | |
| | | | target sequence | | |
| | | | position | | |
| | | | (negative being | | |
| Target | Upstream | Downstream | downstream | Exemplary | |
| Sequence ID | End | End | from TSS) | guide sequence | Strand |
| GD-29250 | 71651407 | 71651429 | −762 | GCAGAATAGCTAGAGCAGCA | + |
| | | | | (SEQ ID NO: 20) | |
| GD-29249 | 71651330 | 71651352 | −685 | GCGCACACCTAATATTTTCA | + |
| | | | | (SEQ ID NO: 19) | |
| GD-29110 | 71652050 | 71652072 | −1405 | GATTTCCTGGCAGGACGCGG | + |
| | | | | (SEQ ID NO: 18) | |
| GD-29109 | 71651984 | 71652006 | −1339 | AAGTTTCTTCAAACACAATG | − |
| | | | | (SEQ ID NO: 17) | |
| GD-29108 | 71651855 | 71651877 | −1210 | GGCGTACCAGCCACTCTGAA | − |
| | | | | (SEQ ID NO: 16) | |
| GD-29106 | 71651682 | 71651704 | −1037 | TTACGCCACGGCTTGAAAGG | + |
| | | | | (SEQ ID NO: 15) | |
| GD-29105 | 71651568 | 71651590 | −923 | CATTTTGCGGACCTGGTGTG | + |
| | | | | (SEQ ID NO: 14) | |
| GD-29104 | 71651497 | 71651519 | −852 | GAGGTTAGGGGAATCCCCCA | − |
| | | | | (SEQ ID NO: 13) | |
| GD-27899 | 71650645 | 71650667 | 0 | GTCGCCGCAGCACCCAGCGC | + |
| | | | | (SEQ ID NO: 21) | |
| GD-27898 | 71650613 | 71650635 | 32 | AGAAGAGTGCCTGCGGCCAG | + |
| | | | | (SEQ ID NO: 22) | |
| GD-27897 | 71650204 | 71650226 | 441 | GTTCCTACTTCATAGGATTG | − |
| | | | | (SEQ ID NO: 7) | |
| GD-27896 | 71650262 | 71650284 | 383 | AGGTTAATTAACTTGCCCTC | + |
| | | | | (SEQ ID NO: 8) | |
| GD-27895 | 71650582 | 71650604 | 63 | GCAGCTAGAGGTTAGACCTC | − |
| | | | | (SEQ ID NO: 12) | |
| GD-27894 | 71650909 | 71650931 | −264 | CGCACGCCGCACGCCTGCGC | + |
| | | | | (SEQ ID NO: 23) | |
| GD-27893 | 71650500 | 71650522 | 145 | GCAAAGCACGGAGTGCAACC | − |
| | | | | (SEQ ID NO: 11) | |
| GD-27892 | 71650815 | 71650837 | −170 | CGATGTCGGTGCGCAGGCCA | − |
| | | | | (SEQ ID NO: 24) | |
| GD-27891 | 71650708 | 71650730 | −63 | CTCGGGCGCCGCGCAGTAGC | + |
| | | | | (SEQ ID NO: 25) | |
| GD-27890 | 71650550 | 71650572 | 95 | ATGCACGAATAGTGCTAAGC | + |
| | | | | (SEQ ID NO: 10) | |
| GD-27889 | 71651006 | 71651028 | −361 | GCGGAAGCGGCCTTGCAACT | − |
| | | | | (SEQ ID NO: 26) | |
| GD-27888 | 71650355 | 71650377 | 290 | CTGCTGTAAACCCATACCGG | + |
| | | | | (SEQ ID NO:9) | |

Effector Moiety

A modulating agent comprises one or more effector moieties which can alter (e.g., increase) the expression of a target gene (e.g., FXN) when localized to an appropriate site in the nucleus of a cell (e.g., by a targeting moiety). In some embodiments, the effector moiety contributes to or enhances the effect of the binding of the modulating agent (e.g., targeting moiety) to the genomic sequence element. In some embodiments, the effector moiety has functionality unrelated to the binding of the targeting moiety. For example, effector moieties may target, e.g., bind, a genomic sequence element or genomic complex component proximal to the genomic sequence element targeted by the targeting moiety, or recruit a transcription factor. As a further example, an effector moiety may comprise an enzymatic activity, e.g., a genetic modification functionality. As a further example, an effector moiety may be or comprise an epigenetic modifying moiety.

In some embodiments, an effector moiety is or comprises a polypeptide. In some embodiments, an effector moiety is or comprises a nucleic acid. In some embodiments, an effector moiety is a chemical, e.g., a chemical that modulates a cytosine (C) or an adenine (A) (e.g., Na bisulfite, ammonium bisulfite). In some embodiments, an effector moiety has enzymatic activity (e.g., methyl transferase, demethylase, nuclease (e.g., Cas9), or deaminase activity). An effector moiety may be or comprise one or more of a small molecule, a peptide, a nucleic acid, a nanoparticle, an aptamer, or a pharmacoagent with poor PK/PD.

In some embodiments, an effector moiety, may comprise a peptide ligand, a full-length protein, a protein fragment, an antibody, an antibody fragment, and/or a targeting aptamer. In some embodiments, the protein may bind a receptor such as an extracellular receptor, neuropeptide, hormone peptide, peptide drug, toxic peptide, viral or microbial peptide, synthetic peptide, or agonist or antagonist peptide.

In some embodiments, an effector moiety may comprise antigens, antibodies, antibody fragments such as, e.g. single domain antibodies, ligands, or receptors such as, e.g., glucagon-like peptide-1 (GLP-1), GLP-2 receptor 2, cholecystokinin B (CCKB), or somatostatin receptor, peptide therapeutics such as, e.g., those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, or degradation or self-destruction peptides such as an apoptosis-inducing peptide signal or photosensitizer peptide.

Peptide or protein moieties for use in effector moieties as described herein may also include small antigen-binding peptides, e.g., antigen binding antibody or antibody-like fragments, such as, e.g., single chain antibodies, nanobodies (see, e.g., Steeland et al. 2016. Nanobodies as therapeutics: big opportunities for small antibodies. Drug Discov Today: 21(7):1076-113). Such small antigen binding peptides may bind, e.g. a cytosolic antigen, a nuclear antigen, an intra-organellar antigen.

In some embodiments, an effector moiety comprises a dominant negative component (e.g., dominant negative moiety), e.g., a protein that recognizes and binds a sequence (e.g., an anchor sequence, e.g., a CTCF binding motif), but with an inactive (e.g., mutated) dimerization domain, e.g., a dimerization domain that is unable to form a functional anchor sequence-mediated conjunction), or binds to a component of a genomic complex (e.g., a transcription factor subunit, etc.) preventing formation of a functional transcription factor, etc. For example, the Zinc Finger domain of CTCF can be altered so that it binds a specific anchor sequence (by adding zinc fingers that recognize flanking nucleic acids), while the homo-dimerization domain is altered to prevent the interaction between engineered CTCF and endogenous forms of CTCF. In some embodiments, a dominant negative component comprises a synthetic nucleating polypeptide with a selected binding affinity for an anchor sequence within a target anchor sequence-mediated conjunction. In some embodiments, binding affinity may be at least 10%, 15%, 20%, 25%, 30%, 35%, 40%, 45%, 50%, 55%, 60%, 65%, 70%, 75%, 80%, 85%, 90%, 95%, or higher or lower than binding affinity of an endogenous nucleating polypeptide (e.g., CTCF) that associates with a target anchor sequence. A synthetic nucleating polypeptide may have between 30-90%, 30-85%, 30-80%, 30-70%, 50-80%, 50-90% amino acid sequence identity to a corresponding endogenous nucleating polypeptide. A nucleating polypeptide may modulate (e.g., disrupt), such as through competitive binding, e.g., competing with binding of an endogenous nucleating polypeptide to its anchor sequence.

In some embodiments, an effector moiety comprises an antibody or fragment thereof. In some embodiments, target gene (e.g., FXN) expression is altered via use of effector moieties that are or comprise one or more antibodies or fragments thereof. In some embodiments, gene expression is altered via use of effector moieties that are or comprise one or more antibodies (or fragments thereof) and dCas9.

In some embodiments, an antibody or fragment thereof for use in an effector moiety may be monoclonal. An antibody may be a fusion, a chimeric antibody, a non-humanized antibody, a partially or fully humanized antibody, etc. As will be understood by one of skill in the art, format of antibody(ies) used may be the same or different depending on a given target.

In some embodiments, an effector moiety, comprises a conjunction nucleating molecule, a nucleic acid encoding a conjunction nucleating molecule, or a combination thereof. A conjunction nucleating molecule may be, e.g., CTCF, cohesin, USF1, YY1, TATA-box binding protein associated factor 3 (TAF3), ZNF143 binding motif, or another polypeptide that promotes formation of an anchor sequence-mediated conjunction. A conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A conjunction nucleating molecule may modulate DNA interactions within or around the anchor sequence-mediated conjunction (e.g., associated with or comprising the genomic sequence element targeted by the targeting moiety). For example, a conjunction nucleating molecule can recruit other factors to an anchor sequence that alters an anchor sequence-mediated conjunction formation or disruption.

A conjunction nucleating molecule may also have a dimerization domain for homo- or heterodimerization. One or more conjunction nucleating molecules, e.g., endogenous and engineered, may interact to form an anchor sequence-mediated conjunction. In some embodiments, a conjunction nucleating molecule is engineered to further include a stabilization domain, e.g., cohesion interaction domain, to stabilize an anchor sequence-mediated conjunction. In some embodiments, a conjunction nucleating molecule is engineered to bind a target sequence, e.g., target sequence binding affinity is modulated. In some embodiments, a conjunction nucleating molecule is selected or engineered with a selected binding affinity for an anchor sequence within an anchor sequence-mediated conjunction.

Conjunction nucleating molecules and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, an effector moiety, comprises a DNA-binding domain of a protein. In some embodiments, a DNA binding domain of an effector moiety enhances or alters targeting of a modulating agent but does not alone achieve complete targeting by a modulating agent (e.g., the targeting moiety is still needed to achieve targeting of the modulating agent). In some embodiments, a DNA binding domain enhances targeting of a modulating agent. In some embodiments, a DNA binding domain enhances efficacy of a modulating agent. DNA-binding proteins have distinct structural motifs, e.g., that play a key role in binding DNA, known to those of skill in the art. In some embodiments, a DNA-binding domain comprises a helix-turn-helix (HTH) motif, a common DNA recognition motif in repressor proteins. Such a motif comprises two helices, one of which recognizes DNA (aka recognition helix) with side chains providing binding specificity. Such motifs are commonly used to regulate proteins that are involved in developmental processes. Sometimes more than one protein competes for the same sequence or recognizes the same DNA fragment. Different proteins may differ in their affinity for the same sequence, or DNA conformation, respectively through H-bonds, salt bridges and Van der Waals interactions.

In some embodiments, a DNA-binding domain comprises a helix-hairpin-helix (HhH) motif. DNA-binding proteins with a HhH structural motif may be involved in non-sequence-specific DNA binding that occurs via the formation of hydrogen bonds between protein backbone nitrogens and DNA phosphate groups.

In some embodiments, a DNA-binding domain comprises a helix-loop-helix (HLH) motif. DNA-binding proteins with an HLH structural motif are transcriptional regulatory proteins and are principally related to a wide array of developmental processes. An HLH structural motif is longer, in terms of residues, than HTH or HhH motifs. Many of these proteins interact to form homo- and hetero-dimers. A structural motif is composed of two long helix regions, with an N-terminal helix binding to DNA, while a complex region allows the protein to dimerize.

In some embodiments, a DNA-binding domain comprises a leucine zipper motif. In some transcription factors, a dimer binding site with DNA forms a leucine zipper. This motif includes two amphipathic helices, one from each subunit, interacting with each other resulting in a left handed coiled-coil super secondary structure. A leucine zipper is an interdigitation of regularly spaced leucine residues in one helix with leucines from an adjacent helix. Mostly, helices involved in leucine zippers exhibit a heptad sequence (abcdefg) with residues a and d being hydrophobic and other residues being hydrophilic. Leucine zipper motifs can mediate either homo- or heterodimer formation.

In some embodiments, a DNA-binding domain comprises a Zn finger domain, where a Zn⁺⁺ ion is coordinated by 2 Cys and 2 His residues. Such a transcription factor includes a trimer with the stoichiometry ββ ′α. An apparent effect of Zn⁺⁺ coordination is stabilization of a small complex structure instead of hydrophobic core residues. Each Zn-finger interacts in a conformationally identical manner with successive triple base pair segments in the major groove of the double helix. Protein-DNA interaction is determined by two factors: (i) H-bonding interaction between α-helix and DNA segment, mostly between Arg residues and Guanine bases. (ii) H-bonding interaction with DNA phosphate backbone, mostly with Arg and His. An alternative Zn-finger motif chelates Zn⁺⁺ with 6 Cys.

In some embodiments, a DNA-binding domain comprises a TATA box binding protein (TBP). TBP was first identified as a component of the class II initiation factor TFIID. These binding proteins participate in transcription by all three nuclear RNA polymerases acting as subunit in each of them. Structure of TBP shows two α/β structural domains of 89-90 amino acids. The C-terminal or core region of TBP binds with high affinity to a TATA consensus sequence (TATAa/tAa/t, SEQ ID NO: 3) recognizing minor groove determinants and promoting DNA bending. TBP resemble a molecular saddle. The binding side is lined with central 8 strands of a 10-stranded anti-parallel (3-sheet. The upper surface contains four α-helices and binds to various components of transcription machinery.

In some embodiments, a DNA-binding domain is or comprises a transcription factor. Transcription factors (TFs) may be modular proteins containing a DNA-binding domain that is responsible for specific recognition of base sequences and one or more effector domains that can activate or repress transcription. TFs interact with chromatin and recruit protein complexes that serve as coactivators or corepressors.

In some embodiments, an effector moiety comprises one or more RNAs (e.g. gRNA) and dCas9. In some embodiments, one or more RNAs is/are targeted to a genomic sequence element via dCas9 and target-specific guide RNA. As will be understood by one of skill in the art, RNAs used for targeting may be the same or different depending on a given target.

An effector moiety may comprise an aptamer, such as an oligonucleotide aptamer or a peptide aptamer. Aptamer moieties are oligonucleotide or peptide aptamers.

An effector moiety may comprise an oligonucleotide aptamer. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers possess desirable storage properties, and elicit little or no immunogenicity in therapeutic applications.

Both DNA and RNA aptamers show robust binding affinities for various targets. For example, DNA and RNA aptamers have been selected for t lysozyme, thrombin, human immunodeficiency virus trans-acting responsive element (HIV TAR), https://en.wikipedia.org/wiki/Aptamer-cite_note-10 hemin, interferon γ, vascular endothelial growth factor (VEGF), prostate specific antigen (PSA), dopamine, and the non-classical oncogene, heat shock factor 1 (HSF1).

Diagnostic techniques for aptamer based plasma protein profiling includes aptamer plasma proteomics. This technology will enable future multi-biomarker protein measurements that can aid diagnostic distinction of disease versus healthy states.

An effector moiety, may comprise a peptide aptamer moiety. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide complexes of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer complex attached to a transcription factor binding domain is screened against a target protein attached to a transcription factor activating domain. In vivo binding of a peptide aptamer to its target via this selection strategy is detected as expression of a downstream yeast marker gene. Such experiments identify particular proteins bound by aptamers, and protein interactions that aptamers disrupt, to cause a given phenotype. In addition, peptide aptamers derivatized with appropriate functional moieties can cause specific post-translational modification of their target proteins, or change subcellular localization of the targets.

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently a yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. Peptides panned from combinatorial peptide libraries have been stored in a special database with named MimoDB.

Exemplary effector moieties include, but are not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as KRAB domain.

Effector Moieties that Affect Genomic Complexes

In some embodiments, a modulating agent comprises an effector moiety that reduces or increases the level of a genomic complex, e.g., an anchor sequence-mediated conjunction, that is associated with or comprises the target gene (e.g., FXN). In some embodiments, the level of a genomic complex (e.g., ASMC) comprising the target gene decreases or increases by at least 10, 20, 30, 40, 50, 60, 70, 80, 90, or 100% (and optionally, up to 100, 90, 80, 70, 60, 50, 40, 30, or 20%) in the presence of a modulating agent comprising the effector moiety relative to the absence of said modulating agent. In some embodiments, the presence of the effector moiety alters, e.g., increases or decreases, occupancy of the genomic complex (e.g., ASMC) at a genomic sequence element operably linked to the target gene (e.g., FXN). In some embodiments, occupancy increases or decreases by at least 10, 20, 30, 40, 50, 60, 70, 80, 90, or 100% (and optionally, up to 100, 90, 80, 70, 60, 50, 40, 30, or 20%) in the presence of a modulating agent comprising the effector moiety relative to the absence of said modulating agent.

In some embodiments, a modulating agent comprises an effector moiety that disrupts an interaction between a genomic sequence element and another genomic complex component or transcription factor. In some embodiments, a modulating agent comprises an effector moiety that decreases the dimerization of an endogenous nucleating polypeptide when present as compared with when the effector moiety is absent.

In some embodiments, an effector moiety alters, e.g., decreases, the expression of a target gene associated with the genomic complex (e.g., ASMC) comprising a targeted component. In some embodiments, the expression of the target gene decreases by at least 10, 20, 30, 40, 50, 60, 70, 80, 90, or 100% (and optionally, up to 100, 90, 80, 70, 60, 50, 40, 30, or 20%) in the presence of a modulating agent comprising the effector moiety relative to the absence of said modulating agent.

In some embodiments, a modulating agent comprises an effector moiety that provides a steric presence (e.g., to inhibit binding of another genomic complex component or transcription factor). An effector moiety may comprise a dominant negative moiety or fragment thereof (e.g., a protein that recognizes and binds a genomic complex component (e.g., a genomic sequence element, e.g., an anchor sequence, (e.g., a CTCF binding motif)) but with an alteration (e.g., mutation) preventing formation of a functional genomic complex (e.g., ASMC)), a polypeptide that interferes with transcription factor binding or function (e.g., contact between a transcription factor and its target sequence to be transcribed), a nucleic acid sequence ligated to a small molecule that imparts steric interference, or any other combination of a recognition element and a steric blocker.

In some embodiments, a modulating agent comprises an effector moiety comprising p65 (also known as RELA), or a functional variant or fragment thereof (e.g., a portion specified by accession number NP_001138610.1). In some embodiments, a modulating agent comprises an effector moiety comprising RTA (the Epstein-Barr virus BRLF1 gene product), or a functional variant or fragment thereof (e.g., a portion specified by accession number AAA66528.1).

Genetic Modifying Moieties

In some embodiments, a modulating agent comprises an effector moiety that is or comprises a genetic modifying moiety (e.g., components of a gene editing system). In some embodiments, a genetic modifying moiety comprises one or more components of a gene editing system. Genetic modifying moieties may be used in a variety of contexts including but not limited to gene editing. For example, a genetic modifying moiety may alter (e.g., introduce a mutation, e.g., a substitution, insertion, or deletion) the sequence of a target gene (e.g., FXN) or a genomic sequence element operably linked to a target gene. As a further example, such moieties may be used to localize an effector moiety to a genetic locus, e.g., so that the modulating agent comprising said effector moiety may physically modify, genetically modify, and/or epigenetically modify a target sequences, e.g., anchor sequence.

In some embodiments, a genetic modifying moiety may target one or more nucleotides, such as through a gene editing system, of a sequence. In some embodiments, a genetic modifying moiety binds a genomic sequence element and alters a genomic complex (e.g., ASMC), e.g., alters topology of an anchor sequence-mediated conjunction, comprising or associated with a target gene (e.g., FXN) and/or a genomic sequence element operably linked to the target gene.

In some embodiments, a genetic modifying moiety targets one or more nucleotides of genomic DNA, e.g., such as through CRISPR, TALEN, dCas9, oligonucleotide pairing, recombination, transposon, within or as a component of a genomic complex (e.g. within an anchor sequence-mediated conjunction) for substitution, addition or deletion.

In some embodiments, a genetic modifying moiety introduces a targeted alteration into one or more nucleotides of genomic DNA wherein the alteration modulates transcription of a gene (e.g., FXN), e.g., in a human cell. A genetic modifying moiety may introduce an alteration into a target gene (e.g., FXN), e.g., into an exon, intron, splice site, or sequence encoding a 5′UTR or 3′UTR. A genetic modifying moiety may introduce an alteration into a genomic sequence element (e.g., promoter or enhancer) operably linked to the target gene (e.g., FXN). A genetic modifying moiety may introduce an alteration into an anchor sequence that participates in an ASMC comprising or associated with the target gene (e.g., FXN) and/or a genomic sequence element operably linked to the target gene. An alteration may include a substitution, addition, or deletion of one or more nucleotides. In some embodiments, a targeted alteration alters at least one of a binding site for a nucleating polypeptide, e.g., altering binding affinity for an anchor sequence within an anchor sequence-mediated conjunction, an alternative splicing site, and a binding site for a non-translated RNA.

In some embodiments, a genetic modifying moiety edits a component of a genomic complex (e.g., a sequence in an anchor sequence-mediated conjunction) via at least one of the following: providing at least one exogenous anchor sequence; an alteration in at least one nucleating polypeptide binding motif, such as by altering binding affinity for a nucleating polypeptide; a change in an orientation of at least one nucleating polypeptide binding motif, such as a CTCF binding motif; and a substitution, addition or deletion in at least one anchor sequence, such as a CTCF binding motif.

Exemplary gene editing systems whose components may be suitable for use in genetic modifying moieties include clustered regulatory interspaced short palindromic repeat (CRISPR) system (e.g., a CRISPR/Cas molecule), zinc finger nucleases (ZFNs) (e.g., a Zn Finger molecule), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 Jul. 30, 46:1-8; and Zheng et al., Precise gene deletion and replacement using the CRISPR/Cas9 system in human cells. BioTechniques, Vol. 57, No. 3, September 2014, pp. 115-124.

For example, in some embodiments, a genetic modifying moiety is site-specific and comprises a Cas nuclease (e.g., Cas9) and a site-specific guide RNA, as described further herein. In some embodiments, a genetic modifying moiety comprises a Cas nuclease (e.g., Cas9) and a site-specific guide RNA. In some embodiments, a Cas nuclease is enzymatically inactive, e.g., a dCas9, as described further herein.

In some embodiments, a genetic modifying moiety may comprise a polypeptide (e.g. peptide or protein moiety) linked to a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to a targeted sequence. Fusions of a catalytically inactive endonuclease, e.g., a dead Cas9 (dCas9, e.g., D10A; H840A) tethered with all or a portion of (e.g., biologically active portion of) an (one or more) effector domain (e.g., epigenome editors including but not restricted to: DNMT3a, DNMT3L, DNMT3b, KRAB domain, Tet1, p300, VP64 and fusions of the aforementioned) create himeric proteins that can be linked to a polypeptide to guide a provided composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

As used herein, a “biologically active portion of an effector domain” is a portion that maintains function (e.g. completely, partially, minimally) of an effector domain (e.g., a “minimal” or “core” domain). In some embodiments, fusion of a dCas9 with all or a portion of one or more effector domains of an epigenetic modifying agent (such as a DNA methylase or enzyme with a role in DNA demethylation, e.g., DNMT3a, DNMT3b, DNMT3L, a DNMT inhibitor, combinations thereof, TET family enzymes, protein acetyl transferase or deacetylase, dCas9-DNMT3a/3L, dCas9-DNMT3a/3L/KRAB, dCas9/VP64) creates a chimeric protein that is linked to the polypeptide and useful in the methods described herein. An effector moiety comprising such a chimeric protein is referred to as either a genetic modifying moiety (because of its use of a gene editing system component, Cas9) or an epigenetic modifying moiety (because of its use of an effector domain of an epigenetic modifying agent).

In some embodiments, provided technologies are described as comprising a gRNA that specifically targets a target gene. In some embodiments, the target gene is an oncogene, a tumor suppressor, or a nucleotide repeat disease related gene.

In some embodiments, technologies provided herein include methods of delivering one or more genetic modifying moieties (e.g., CRISPR system components) described herein to a subject, e.g., to a nucleus of a cell or tissue of a subject, by linking such a moiety to a targeting moiety as part of a fusion molecule.

Epigenetic Modifying Moieties

In some embodiments, an effector moiety is or comprises an epigenetic modifying moiety that modulates the structure of chromatin or alters an epigenetic marker (e.g., one or more of DNA methylation, histone methylation, histone acetylation, histone sumoylation, histone phosphorylation, and RNA-associated silencing).

Epigenetic modifying moieties useful in methods and compositions of the present disclosure include agents that affect, e.g., DNA methylation, histone acetylation, and RNA-associated silencing. In some embodiments, methods provided herein involve sequence-specific targeting (e.g., via a modulating agent comprising a targeting moiety that specifically binds a target sequence) of an epigenetic enzyme (e.g., an enzyme that generates or removes epigenetic marks, e.g., acetylation and/or methylation). Exemplary epigenetic enzymes that can be targeted to a genomic sequence element as described herein include DNA demethylases (e.g., the TET family), histone methyltransferases, histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an epigenetic modifying moiety comprises a histone methyltransferase activity (e.g., a protein chosen from DOT1L, PRDM9, PRMT1, PRMT2, PRMT3, PRMT4, PRMT5, NSD1, NSD2, NSD3, or a functional variant or fragment of any thereof. In some embodiments, an epigenetic modifying moiety comprises a histone acetyltransferase activity (e.g., a protein chosen from p300, CREB-binding protein (CBP), or a functional variant or fragment of any thereof). In some embodiments, an epigenetic modifying moiety comprises a DNA demethylase activity (e.g., a protein chosen from TET1, TET2, TET3, or TDG, or a functional variant or fragment of any thereof). In some embodiments, an epigenetic modifying moiety comprises a transcription activator activity (e.g., a protein chosen from VP16, VP64, VP160, VPR, or a functional variant or fragment of any thereof).

In some embodiments, an epigenetic modifying moiety useful herein comprises a construct described in Koferle et al. Genome Medicine 7.59 (2015):1-3 (e.g., at Table 1), incorporated herein by reference. For example, in some embodiments, an expression repressor comprises or is a construct found in Table 1 of Koferle et al., e.g., a histone acetyltransferase, histone deacetylase, histone methyltransferase, DNA demethylation, or H3K4 and/or H3K9 histone demethylase described in Table 1 (e.g., dCas9-p300, TALE-TET1).

In some embodiments, an epigenetic modifying moiety comprises a histone demethylase activity (e.g., a protein chosen from KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof). In some embodiments, an epigenetic modifying moiety comprises a histone deacetylase activity (e.g., a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, an epigenetic modifying moiety comprises a DNA methyltransferase activity (e.g., a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof). In some embodiments, an epigenetic modifying moiety comprises a transcription repressor activity (e.g., a protein chosen from KRAB, MeCP2, HP1, RBBP4, REST, FOG1, SUZ12, or a functional variant or fragment of any thereof).

Exemplary Modulating Agents

In some embodiments, a modulating agent comprises a targeting moiety comprising a CRISPR/Cas molecule and an effector moiety comprising a histone acetyltransferase activity, e.g., p300 or a functional fragment or variant thereof. In some embodiments, a modulating agent comprises a targeting moiety comprising a catalytically inactive Cas9 molecule (e.g., a dCas9) and an effector moiety comprising p300 or a functional fragment or variant thereof.

In some embodiments, a modulating agent is encoded by the nucleic acid sequence of SEQ ID NO: 300 or a sequence with at least 80, 85, 90, 95, 96, 97, 98, or 99% identity to said sequence. In some embodiments, a modulating agent is encoded by the nucleic acid sequence of SEQ ID NO: 308 or a sequence with at least 80, 85, 90, 95, 96, 97, 98, or 99% identity to said sequence. In some embodiments, a modulating agent comprises an amino acid sequence of SEQ ID NO: 304 or an amino acid sequence encoded by the nucleic acid sequence of either SEQ ID NOs: 300 or 308, or an amino acid sequence with at least 80, 85, 90, 95, 96, 97, 98, or 99% identity to either of the same.

dCas9-p300 exemplary encoding sequence 1
(SEQ ID NO: 300)
AGGAAATAAGAGAGAAAAGAAGAGTAAGAAGAAATATAAGAGCCACCATGGCCCCCAAGAAGAAGCGGAAGGTGGGC
ATCCACGGCGTGCCCGCCGCCGACAAGAAGTACAGCATCGGCCTGGCCATCGGCACCAACAGCGTGGGCTGGGCCGT
GATCACCGACGAGTACAAGGTGCCCAGCAAGAAGTTCAAGGTGCTGGGCAACACCGACCGGCACAGCATCAAGAAGA
ACCTGATCGGCGCCCTGCTGTTCGACAGCGGCGAGACCGCCGAGGCCACCCGGCTGAAGCGGACCGCCCGGCGGCGG
TACACCCGGCGGAAGAACCGGATCTGCTACCTGCAGGAGATCTTCAGCAACGAGATGGCCAAGGTGGACGACAGCTT
CTTCCACCGGCTGGAGGAGAGCTTCCTGGTGGAGGAGGACAAGAAGCACGAGCGGCACCCCATCTTCGGCAACATCG
TGGACGAGGTGGCCTACCACGAGAAGTACCCCACCATCTACCACCTGCGGAAGAAGCTGGTGGACAGCACCGACAAG
GCCGACCTGCGGCTGATCTACCTGGCCCTGGCCCACATGATCAAGTTCCGGGGCCACTTCCTGATCGAGGGCGACCT
GAACCCCGACAACAGCGACGTGGACAAGCTGTTCATCCAGCTGGTGCAGACCTACAACCAGCTGTTCGAGGAGAACC
CCATCAACGCCAGCGGCGTGGACGCCAAGGCCATCCTGAGCGCCCGGCTGAGCAAGAGCCGGCGGCTGGAGAACCTG
ATCGCCCAGCTGCCCGGCGAGAAGAAGAACGGCCTGTTCGGCAACCTGATCGCCCTGAGCCTGGGCCTGACCCCCAA
CTTCAAGAGCAACTTCGACCTGGCCGAGGACGCCAAGCTGCAGCTGAGCAAGGACACCTACGACGACGACCTGGACA
ACCTGCTGGCCCAGATCGGCGACCAGTACGCCGACCTGTTCCTGGCCGCCAAGAACCTGAGCGACGCCATCCTGCTG
AGCGACATCCTGCGGGTGAACACCGAGATCACCAAGGCCCCCCTGAGCGCCAGCATGATCAAGCGGTACGACGAGCA
CCACCAGGACCTGACCCTGCTGAAGGCCCTGGTGCGGCAGCAGCTGCCCGAGAAGTACAAGGAGATCTTCTTCGACC
AGAGCAAGAACGGCTACGCCGGCTACATCGACGGCGGCGCCAGCCAGGAGGAGTTCTACAAGTTCATCAAGCCCATC
CTGGAGAAGATGGACGGCACCGAGGAGCTGCTGGTGAAGCTGAACCGGGAGGACCTGCTGCGGAAGCAGCGGACCTT
CGACAACGGCAGCATCCCCCACCAGATCCACCTGGGCGAGCTGCACGCCATCCTGCGGCGGCAGGAGGACTTCTACC
CCTTCCTGAAGGACAACCGGGAGAAGATCGAGAAGATCCTGACCTTCCGGATCCCCTACTACGTGGGCCCCCTGGCC
CGGGGCAACAGCCGGTTCGCCTGGATGACCCGGAAATCCGAGGAGACCATCACCCCCTGGAACTTCGAGGAGGTGGT
GGACAAGGGCGCCAGCGCCCAGAGCTTCATCGAGCGGATGACCAACTTCGACAAGAACCTGCCCAACGAGAAGGTGC
TGCCCAAGCACAGCCTGCTGTACGAGTACTTCACCGTGTACAACGAGCTGACCAAGGTGAAGTACGTGACCGAGGGC
ATGCGGAAGCCCGCCTTCCTGAGCGGCGAGCAGAAGAAGGCCATCGTGGACCTGCTGTTCAAGACCAACCGGAAGGT
GACCGTGAAGCAGCTGAAGGAGGACTACTTCAAGAAGATCGAGTGCTTCGACAGCGTGGAGATCAGCGGCGTGGAGG
ACCGGTTCAACGCCAGCCTGGGCACCTACCACGACCTGCTGAAGATCATCAAGGACAAGGACTTCCTGGACAACGAG
GAGAACGAGGACATCCTGGAGGACATCGTGCTGACCCTGACCCTGTTCGAGGACCGGGAGATGATCGAGGAGCGGCT
GAAAACCTACGCCCACCTGTTCGACGACAAGGTGATGAAGCAGCTGAAGCGGCGGCGGTACACCGGCTGGGGCCGGC
TGAGCCGGAAGCTGATCAACGGCATCCGGGACAAGCAGAGCGGCAAGACCATCCTGGACTTCCTGAAATCCGACGGC
TTCGCCAACCGGAACTTCATGCAGCTGATCCACGACGACAGCCTGACCTTCAAGGAGGACATCCAGAAGGCCCAGGT
GAGCGGCCAGGGCGACAGCCTGCACGAGCACATCGCCAACCTGGCCGGCAGCCCCGCCATCAAGAAGGGCATCCTGC
AGACCGTGAAGGTGGTGGACGAGCTGGTGAAGGTGATGGGCCGGCACAAGCCCGAGAACATCGTGATCGAGATGGCC
CGGGAGAACCAGACCACCCAGAAGGGCCAGAAGAACAGCCGGGAGCGGATGAAGCGGATCGAGGAGGGCATCAAGGA
GCTGGGCAGCCAGATCCTGAAGGAGCACCCCGTGGAGAACACCCAGCTGCAGAACGAGAAGCTGTACCTGTACTACC
TGCAGAACGGCCGGGACATGTACGTGGACCAGGAGCTGGACATCAACCGGCTGAGCGACTACGACGTGGCCGCCATC
GTGCCCCAGAGCTTCCTGAAGGACGACAGCATCGACAACAAGGTGCTGACCCGGAGCGACAAGGCCCGGGGCAAGAG
CGACAACGTGCCCAGCGAGGAGGTGGTGAAGAAGATGAAGAACTACTGGCGGCAGCTGCTGAACGCCAAGCTGATCA
CCCAGCGGAAGTTCGACAACCTGACCAAGGCCGAGCGGGGCGGCCTGAGCGAGCTGGACAAGGCCGGCTTCATCAAG
CGGCAGCTGGTGGAGACCCGGCAGATCACCAAGCACGTGGCCCAGATCCTGGACAGCCGGATGAACACCAAGTACGA
CGAGAACGACAAGCTGATCCGGGAGGTGAAGGTGATCACCCTGAAATCCAAGCTGGTGAGCGACTTCCGGAAGGACT
TCCAGTTCTACAAGGTGCGGGAGATCAACAACTACCACCACGCCCACGACGCCTACCTGAACGCCGTGGTGGGCACC
GCCCTGATCAAGAAGTACCCCAAGCTGGAGAGCGAGTTCGTGTACGGCGACTACAAGGTGTACGACGTGCGGAAGAT
GATCGCCAAGAGCGAGCAGGAGATCGGCAAGGCCACCGCCAAGTACTTCTTCTACAGCAACATCATGAACTTCTTCA
AGACCGAGATCACCCTGGCCAACGGCGAGATCCGGAAGCGGCCCCTGATCGAGACCAACGGCGAGACCGGCGAGATC
GTGTGGGACAAGGGCCGGGACTTCGCCACCGTGCGGAAGGTGCTGAGCATGCCCCAGGTGAACATCGTGAAGAAAAC
CGAGGTGCAGACCGGCGGCTTCAGCAAGGAGAGCATCCTGCCCAAGCGGAACAGCGACAAGCTGATCGCCCGGAAGA
AGGACTGGGACCCCAAGAAGTACGGCGGCTTCGACAGCCCCACCGTGGCCTACAGCGTGCTGGTGGTGGCCAAGGTG
GAGAAGGGCAAGAGCAAGAAGCTGAAATCCGTGAAGGAGCTGCTGGGCATCACCATCATGGAGCGGAGCAGCTTCGA
GAAGAACCCCATCGACTTCCTGGAGGCCAAGGGCTACAAGGAGGTGAAGAAGGACCTGATCATCAAGCTGCCCAAGT
ACAGCCTGTTCGAGCTGGAGAACGGCCGGAAGCGGATGCTGGCCAGCGCCGGCGAGCTGCAGAAGGGCAACGAGCTG
GCCCTGCCCAGCAAGTACGTGAACTTCCTGTACCTGGCCAGCCACTACGAGAAGCTGAAGGGCAGCCCCGAGGACAA
CGAGCAGAAGCAGCTGTTCGTGGAGCAGCACAAGCACTACCTGGACGAGATCATCGAGCAGATCAGCGAGTTCAGCA
AGCGGGTGATCCTGGCCGACGCCAACCTGGACAAGGTGCTGAGCGCCTACAACAAGCACCGGGACAAGCCCATCCGG
GAGCAGGCCGAGAACATCATCCACCTGTTCACCCTGACCAACCTGGGCGCCCCCGCCGCCTTCAAGTACTTCGACAC
CACCATCGACCGGAAGCGGTACACCAGCACCAAGGAGGTGCTGGACGCCACCCTGATCCACCAGAGCATCACCGGCC
TGTACGAGACCCGGATCGACCTGAGCCAGCTGGGCGGCGACAAGCGGCCCGCCGCCACCAAGAAGGCCGGCCAGGCC
AAGAAGAAGAAGGGCCGGGCCATCTTCAAGCCCGAGGAGCTGCGGCAGGCCCTGATGCCCACCCTGGAGGCCCTGTA
CCGGCAGGACCCCGAGAGCCTGCCCTTCCGGCAGCCCGTGGACCCCCAGCTGCTGGGCATCCCCGACTACTTCGACA
TCGTGAAATCCCCCATGGACCTGAGCACCATCAAGCGGAAGCTGGACACCGGCCAGTACCAGGAGCCCTGGCAGTAC
GTGGACGACATCTGGCTGATGTTCAACAACGCCTGGCTGTACAACCGGAAAACCAGCCGGGTGTACAAGTACTGCAG
CAAGCTGAGCGAGGTGTTCGAGCAGGAGATCGACCCCGTGATGCAGAGCCTGGGCTACTGCTGCGGCCGGAAGCTGG
AGTTCAGCCCCCAGACCCTGTGCTGCTACGGCAAGCAGCTGTGCACCATCCCCCGGGACGCCACCTACTACAGCTAC
CAGAACCGGTACCACTTCTGCGAGAAGTGCTTCAACGAGATCCAGGGCGAGAGCGTGAGCCTGGGCGACGACCCCAG
CCAGCCCCAGACCACCATCAACAAGGAGCAGTTCAGCAAGCGGAAGAACGACACCCTGGACCCCGAGCTGTTCGTGG
AGTGCACCGAGTGCGGCCGGAAGATGCACCAGATCTGCGTGCTGCACCACGAGATCATCTGGCCCGCCGGCTTCGTG
TGCGACGGCTGCCTGAAGAAATCCGCCCGGACCCGGAAGGAGAACAAGTTCAGCGCCAAGCGGCTGCCCAGCACCCG
GCTGGGCACCTTCCTGGAGAACCGGGTGAACGACTTCCTGCGGCGGCAGAACCACCCCGAGAGCGGCGAGGTGACCG
TGCGGGTGGTGCACGCCAGCGACAAGACCGTGGAGGTGAAGCCCGGCATGAAGGCCCGGTTCGTGGACAGCGGCGAG
ATGGCCGAGAGCTTCCCCTACCGGACCAAGGCCCTGTTCGCCTTCGAGGAGATCGACGGCGTGGACCTGTGCTTCTT
CGGCATGCACGTGCAGGAGTACGGCAGCGACTGCCCCCCCCCCAACCAGCGGCGGGTGTACATCAGCTACCTGGACA
GCGTGCACTTCTTCCGGCCCAAGTGCCTGCGGACCGCCGTGTACCACGAGATCCTGATCGGCTACCTGGAGTACGTG
AAGAAGCTGGGCTACACCACCGGCCACATCTGGGCCTGCCCCCCCAGCGAGGGCGACGACTACATCTTCCACTGCCA
CCCCCCCGACCAGAAGATCCCCAAGCCCAAGCGGCTGCAGGAGTGGTACAAGAAGATGCTGGACAAGGCCGTGAGCG
AGCGGATCGTGCACGACTACAAGGACATCTTCAAGCAGGCCACCGAGGACCGGCTGACCAGCGCCAAGGAGCTGCCC
TACTTCGAGGGCGACTTCTGGCCCAACGTGCTGGAGGAGAGCATCAAGGAGCTGGAGCAGGAGGAGGAGGAGCGGAA
GCGGGAGGAGAACACCAGCAACGAGAGCACCGACGTGACCAAGGGCGACAGCAAGAACGCCAAGAAGAAGAACAACA
AGAAAACCAGCAAGAACAAGAGCAGCCTGAGCCGGGGCAACAAGAAGAAGCCCGGCATGCCCAACGTGAGCAACGAC
CTGAGCCAGAAGCTGTACGCCACCATGGAGAAGCACAAGGAGGTGTTCTTCGTGATCCGGCTGATCGCCGGCCCCGC
CGCCAACAGCCTGCCCCCCATCGTGGACCCCGACCCCCTGATCCCCTGCGACCTGATGGACGGCCGGGACGCCTTCC
TGACCCTGGCCCGGGACAAGCACCTGGAGTTCAGCAGCCTGCGGCGGGCCCAGTGGAGCACCATGTGCATGCTGGTG
GAGCTGCACACCCAGAGCCAGGACAGCGGCGGCAAGCGGCCCGCCGCCACCAAGAAGGCCGGCCAGGCCAAGAAGAA
GAAGGGCAGCTACCCCTACGACGTGCCCGACTACGCCTGAGCGGCCGCTTAATTAAGCTGCCTTCTGCGGGGCTTGC
CTTCTGGCCATGCCCTTCTTCTCTCCCTTGCACCTGTACCTCTTGGTCTTTGAATAAAGCCTGAGTAGGAAGTCTAG
AAAAAAAAAAAAAAAAAAAAAAAAAAAAAAAAAAAAAAAAAAAAAAAAAAAAAAAAAAAAAAAAAAAAAAAAAAAAA
AAA
dCas9-p300 exemplary encoding sequence 2
(SEQ ID NO: 308)
ATGGCCCCCAAGAAGAAGCGGAAGGTGGGCATCCACGGCGTGCCCGCCGCCGACAAGAAGTACAGCATCGGCCTGGC
CATCGGCACCAACAGCGTGGGCTGGGCCGTGATCACCGACGAGTACAAGGTGCCCAGCAAGAAGTTCAAGGTGCTGG
GCAACACCGACCGGCACAGCATCAAGAAGAACCTGATCGGCGCCCTGCTGTTCGACAGCGGCGAGACCGCCGAGGCC
ACCCGGCTGAAGCGGACCGCCCGGCGGCGGTACACCCGGCGGAAGAACCGGATCTGCTACCTGCAGGAGATCTTCAG
CAACGAGATGGCCAAGGTGGACGACAGCTTCTTCCACCGGCTGGAGGAGAGCTTCCTGGTGGAGGAGGACAAGAAGC
ACGAGCGGCACCCCATCTTCGGCAACATCGTGGACGAGGTGGCCTACCACGAGAAGTACCCCACCATCTACCACCTG
CGGAAGAAGCTGGTGGACAGCACCGACAAGGCCGACCTGCGGCTGATCTACCTGGCCCTGGCCCACATGATCAAGTT
CCGGGGCCACTTCCTGATCGAGGGCGACCTGAACCCCGACAACAGCGACGTGGACAAGCTGTTCATCCAGCTGGTGC
AGACCTACAACCAGCTGTTCGAGGAGAACCCCATCAACGCCAGCGGCGTGGACGCCAAGGCCATCCTGAGCGCCCGG
CTGAGCAAGAGCCGGCGGCTGGAGAACCTGATCGCCCAGCTGCCCGGCGAGAAGAAGAACGGCCTGTTCGGCAACCT
GATCGCCCTGAGCCTGGGCCTGACCCCCAACTTCAAGAGCAACTTCGACCTGGCCGAGGACGCCAAGCTGCAGCTGA
GCAAGGACACCTACGACGACGACCTGGACAACCTGCTGGCCCAGATCGGCGACCAGTACGCCGACCTGTTCCTGGCC
GCCAAGAACCTGAGCGACGCCATCCTGCTGAGCGACATCCTGCGGGTGAACACCGAGATCACCAAGGCCCCCCTGAG
CGCCAGCATGATCAAGCGGTACGACGAGCACCACCAGGACCTGACCCTGCTGAAGGCCCTGGTGCGGCAGCAGCTGC
CCGAGAAGTACAAGGAGATCTTCTTCGACCAGAGCAAGAACGGCTACGCCGGCTACATCGACGGCGGCGCCAGCCAG
GAGGAGTTCTACAAGTTCATCAAGCCCATCCTGGAGAAGATGGACGGCACCGAGGAGCTGCTGGTGAAGCTGAACCG
GGAGGACCTGCTGCGGAAGCAGCGGACCTTCGACAACGGCAGCATCCCCCACCAGATCCACCTGGGCGAGCTGCACG
CCATCCTGCGGCGGCAGGAGGACTTCTACCCCTTCCTGAAGGACAACCGGGAGAAGATCGAGAAGATCCTGACCTTC
CGGATCCCCTACTACGTGGGCCCCCTGGCCCGGGGCAACAGCCGGTTCGCCTGGATGACCCGGaaatccGAGGAGAC
CATCACCCCCTGGAACTTCGAGGAGGTGGTGGACAAGGGCGCCAGCGCCCAGAGCTTCATCGAGCGGATGACCAACT
TCGACAAGAACCTGCCCAACGAGAAGGTGCTGCCCAAGCACAGCCTGCTGTACGAGTACTTCACCGTGTACAACGAG
CTGACCAAGGTGAAGTACGTGACCGAGGGCATGCGGAAGCCCGCCTTCCTGAGCGGCGAGCAGAAGAAGGCCATCGT
GGACCTGCTGTTCAAGACCAACCGGAAGGTGACCGTGAAGCAGCTGAAGGAGGACTACTTCAAGAAGATCGAGTGCT
TCGACAGCGTGGAGATCAGCGGCGTGGAGGACCGGTTCAACGCCAGCCTGGGCACCTACCACGACCTGCTGAAGATC
ATCAAGGACAAGGACTTCCTGGACAACGAGGAGAACGAGGACATCCTGGAGGACATCGTGCTGACCCTGACCCTGTT
CGAGGACCGGGAGATGATCGAGGAGCGGCTGAAaACCTACGCCCACCTGTTCGACGACAAGGTGATGAAGCAGCTGA
AGCGGCGGCGGTACACCGGCTGGGGCCGGCTGAGCCGGAAGCTGATCAACGGCATCCGGGACAAGCAGAGCGGCAAG
ACCATCCTGGACTTCCTGaaatccGACGGCTTCGCCAACCGGAACTTCATGCAGCTGATCCACGACGACAGCCTGAC
CTTCAAGGAGGACATCCAGAAGGCCCAGGTGAGCGGCCAGGGCGACAGCCTGCACGAGCACATCGCCAACCTGGCCG
GCAGCCCCGCCATCAAGAAGGGCATCCTGCAGACCGTGAAGGTGGTGGACGAGCTGGTGAAGGTGATGGGCCGGCAC
AAGCCCGAGAACATCGTGATCGAGATGGCCCGGGAGAACCAGACCACCCAGAAGGGCCAGAAGAACAGCCGGGAGCG
GATGAAGCGGATCGAGGAGGGCATCAAGGAGCTGGGCAGCCAGATCCTGAAGGAGCACCCCGTGGAGAACACCCAGC
TGCAGAACGAGAAGCTGTACCTGTACTACCTGCAGAACGGCCGGGACATGTACGTGGACCAGGAGCTGGACATCAAC
CGGCTGAGCGACTACGACGTGGCCGCCATCGTGCCCCAGAGCTTCCTGAAGGACGACAGCATCGACAACAAGGTGCT
GACCCGGAGCGACAAGGCCCGGGGCAAGAGCGACAACGTGCCCAGCGAGGAGGTGGTGAAGAAGATGAAGAACTACT
GGCGGCAGCTGCTGAACGCCAAGCTGATCACCCAGCGGAAGTTCGACAACCTGACCAAGGCCGAGCGGGGCGGCCTG
AGCGAGCTGGACAAGGCCGGCTTCATCAAGCGGCAGCTGGTGGAGACCCGGCAGATCACCAAGCACGTGGCCCAGAT
CCTGGACAGCCGGATGAACACCAAGTACGACGAGAACGACAAGCTGATCCGGGAGGTGAAGGTGATCACCCTGaaat
ccAAGCTGGTGAGCGACTTCCGGAAGGACTTCCAGTTCTACAAGGTGCGGGAGATCAACAACTACCACCACGCCCAC
GACGCCTACCTGAACGCCGTGGTGGGCACCGCCCTGATCAAGAAGTACCCCAAGCTGGAGAGCGAGTTCGTGTACGG
CGACTACAAGGTGTACGACGTGCGGAAGATGATCGCCAAGAGCGAGCAGGAGATCGGCAAGGCCACCGCCAAGTACT
TCTTCTACAGCAACATCATGAACTTCTTCAAGACCGAGATCACCCTGGCCAACGGCGAGATCCGGAAGCGGCCCCTG
ATCGAGACCAACGGCGAGACCGGCGAGATCGTGTGGGACAAGGGCCGGGACTTCGCCACCGTGCGGAAGGTGCTGAG
CATGCCCCAGGTGAACATCGTGAAGAAaACCGAGGTGCAGACCGGCGGCTTCAGCAAGGAGAGCATCCTGCCCAAGC
GGAACAGCGACAAGCTGATCGCCCGGAAGAAGGACTGGGACCCCAAGAAGTACGGCGGCTTCGACAGCCCCACCGTG
GCCTACAGCGTGCTGGTGGTGGCCAAGGTGGAGAAGGGCAAGAGCAAGAAGCTGaaatccGTGAAGGAGCTGCTGGG
CATCACCATCATGGAGCGGAGCAGCTTCGAGAAGAACCCCATCGACTTCCTGGAGGCCAAGGGCTACAAGGAGGTGA
AGAAGGACCTGATCATCAAGCTGCCCAAGTACAGCCTGTTCGAGCTGGAGAACGGCCGGAAGCGGATGCTGGCCAGC
GCCGGCGAGCTGCAGAAGGGCAACGAGCTGGCCCTGCCCAGCAAGTACGTGAACTTCCTGTACCTGGCCAGCCACTA
CGAGAAGCTGAAGGGCAGCCCCGAGGACAACGAGCAGAAGCAGCTGTTCGTGGAGCAGCACAAGCACTACCTGGACG
AGATCATCGAGCAGATCAGCGAGTTCAGCAAGCGGGTGATCCTGGCCGACGCCAACCTGGACAAGGTGCTGAGCGCC
TACAACAAGCACCGGGACAAGCCCATCCGGGAGCAGGCCGAGAACATCATCCACCTGTTCACCCTGACCAACCTGGG
CGCCCCCGCCGCCTTCAAGTACTTCGACACCACCATCGACCGGAAGCGGTACACCAGCACCAAGGAGGTGCTGGACG
CCACCCTGATCCACCAGAGCATCACCGGCCTGTACGAGACCCGGATCGACCTGAGCCAGCTGGGCGGCGACAAGCGG
CCCGCCGCCACCAAGAAGGCCGGCCAGGCCAAGAAGAAGAAGGGCCGGGCCATCTTCAAGCCCGAGGAGCTGCGGCA
GGCCCTGATGCCCACCCTGGAGGCCCTGTACCGGCAGGACCCCGAGAGCCTGCCCTTCCGGCAGCCCGTGGACCCCC
AGCTGCTGGGCATCCCCGACTACTTCGACATCGTGaaatccCCCATGGACCTGAGCACCATCAAGCGGAAGCTGGAC
ACCGGCCAGTACCAGGAGCCCTGGCAGTACGTGGACGACATCTGGCTGATGTTCAACAACGCCTGGCTGTACAACCG
GAAaACCAGCCGGGTGTACAAGTACTGCAGCAAGCTGAGCGAGGTGTTCGAGCAGGAGATCGACCCCGTGATGCAGA
GCCTGGGCTACTGCTGCGGCCGGAAGCTGGAGTTCAGCCCCCAGACCCTGTGCTGCTACGGCAAGCAGCTGTGCACC
ATCCCCCGGGACGCCACCTACTACAGCTACCAGAACCGGTACCACTTCTGCGAGAAGTGCTTCAACGAGATCCAGGG
CGAGAGCGTGAGCCTGGGCGACGACCCCAGCCAGCCCCAGACCACCATCAACAAGGAGCAGTTCAGCAAGCGGAAGA
ACGACACCCTGGACCCCGAGCTGTTCGTGGAGTGCACCGAGTGCGGCCGGAAGATGCACCAGATCTGCGTGCTGCAC
CACGAGATCATCTGGCCCGCCGGCTTCGTGTGCGACGGCTGCCTGAAGaaatccGCCCGGACCCGGAAGGAGAACAA
GTTCAGCGCCAAGCGGCTGCCCAGCACCCGGCTGGGCACCTTCCTGGAGAACCGGGTGAACGACTTCCTGCGGCGGC
AGAACCACCCCGAGAGCGGCGAGGTGACCGTGCGGGTGGTGCACGCCAGCGACAAGACCGTGGAGGTGAAGCCCGGC
ATGAAGGCCCGGTTCGTGGACAGCGGCGAGATGGCCGAGAGCTTCCCCTACCGGACCAAGGCCCTGTTCGCCTTCGA
GGAGATCGACGGCGTGGACCTGTGCTTCTTCGGCATGCACGTGCAGGAGTACGGCAGCGACTGCCCCCCCCCCAACC
AGCGGCGGGTGTACATCAGCTACCTGGACAGCGTGCACTTCTTCCGGCCCAAGTGCCTGCGGACCGCCGTGTACCAC
GAGATCCTGATCGGCTACCTGGAGTACGTGAAGAAGCTGGGCTACACCACCGGCCACATCTGGGCCTGCCCCCCCAG
CGAGGGCGACGACTACATCTTCCACTGCCACCCCCCCGACCAGAAGATCCCCAAGCCCAAGCGGCTGCAGGAGTGGT
ACAAGAAGATGCTGGACAAGGCCGTGAGCGAGCGGATCGTGCACGACTACAAGGACATCTTCAAGCAGGCCACCGAG
GACCGGCTGACCAGCGCCAAGGAGCTGCCCTACTTCGAGGGCGACTTCTGGCCCAACGTGCTGGAGGAGAGCATCAA
GGAGCTGGAGCAGGAGGAGGAGGAGCGGAAGCGGGAGGAGAACACCAGCAACGAGAGCACCGACGTGACCAAGGGCG
ACAGCAAGAACGCCAAGAAGAAGAACAACAAGAAaACCAGCAAGAACAAGAGCAGCCTGAGCCGGGGCAACAAGAAG
AAGCCCGGCATGCCCAACGTGAGCAACGACCTGAGCCAGAAGCTGTACGCCACCATGGAGAAGCACAAGGAGGTGTT
CTTCGTGATCCGGCTGATCGCCGGCCCCGCCGCCAACAGCCTGCCCCCCATCGTGGACCCCGACCCCCTGATCCCCT
GCGACCTGATGGACGGCCGGGACGCCTTCCTGACCCTGGCCCGGGACAAGCACCTGGAGTTCAGCAGCCTGCGGCGG
GCCCAGTGGAGCACCATGTGCATGCTGGTGGAGCTGCACACCCAGAGCCAGGACAGCGGCGGCAAGCGGCCCGCCGC
CACCAAGAAGGCCGGCCAGGCCAAGAAGAAGAAGGGCAGCTACCCCTACGACGTGCCCGACTACGCCTGA
MAPKKKRKVGIHGVPAADKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFLKDNREKIEKILTF
RIPYYVGPLARGNSRFAWMTRKSEETITPWNFEEVVDKGASAQSFIERMTNFDKNLPNEKVLPKHSLLYEYFTVYNE
(SEQ ID NO: 304)
LTKVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRIDLSQLGGDKR
PAATKKAGQAKKKKGRAIFKPEELRQALMPTLEALYRQDPESLPFRQPVDPQLLGIPDYFDIVKSPMDLSTIKRKLD
TGQYQEPWQYVDDIWLMFNNAWLYNRKTSRVYKYCSKLSEVFEQEIDPVMQSLGYCCGRKLEFSPQTLCCYGKQLCT
IPRDATYYSYQNRYHFCEKCFNEIQGESVSLGDDPSQPQTTINKEQFSKRKNDTLDPELFVECTECGRKMHQICVLH
HEIIWPAGFVCDGCLKKSARTRKENKFSAKRLPSTRLGTFLENRVNDFLRRQNHPESGEVTVRVVHASDKTVEVKPG
MKARFVDSGEMAESFPYRTKALFAFEEIDGVDLCFFGMHVQEYGSDCPPPNQRRVYISYLDSVHFFRPKCLRTAVYH
EILIGYLEYVKKLGYTTGHIWACPPSEGDDYIFHCHPPDQKIPKPKRLQEWYKKMLDKAVSERIVHDYKDIFKQATE
DRLTSAKELPYFEGDFWPNVLEESIKELEQEEEERKREENTSNESTDVTKGDSKNAKKKNNKKTSKNKSSLSRGNKK
KPGMPNVSNDLSQKLYATMEKHKEVFFVIRLIAGPAANSLPPIVDPDPLIPCDLMDGRDAFLTLARDKHLEFSSLRR
AQWSTMCMLVELHTQSQDSGGKRPAATKKAGQAKKKKGSYPYDVPDYA

In some embodiments, a modulating agent comprises a targeting moiety comprising a CRISPR/Cas molecule and an effector moiety comprising a transcription activator activity, e.g., VP64 or a functional fragment or variant thereof. In some embodiments, a modulating agent comprises a targeting moiety comprising a catalytically inactive Cas9 molecule (e.g., a dCas9) and an effector moiety comprising VP64 or a functional fragment or variant thereof.

In some embodiments, a modulating agent is encoded by the nucleic acid sequence of SEQ ID NO: 301 or a sequence with at least 80, 85, 90, 95, 96, 97, 98, or 99% identity to said sequence. In some embodiments, a modulating agent is encoded by the nucleic acid sequence of SEQ ID NO: 309 or a sequence with at least 80, 85, 90, 95, 96, 97, 98, or 99% identity to said sequence. In some embodiments, a modulating agent comprises an amino acid sequence of SEQ ID NO: 305 or an amino acid sequence encoded by the nucleic acid sequence of either of SEQ ID NOs: 301 or 309, or an amino acid sequence with at least 80, 85, 90, 95, 96, 97, 98, or 99% identity to either of the same.

dCas9-VPR (VP64-p65-RTA) exemplary sequence 1
(SEQ ID NO: 301)
AGGAAATAAGAGAGAAAAGAAGAGTAAGAAGAAATATAAGAGCCACCATGGCCCCCAAGAAGAAGCGGAAGGTGGGC
ATCCACGGCGTGCCCGCCGCCGACAAGAAGTACAGCATCGGCCTGGCCATCGGCACCAACAGCGTGGGCTGGGCCGT
GATCACCGACGAGTACAAGGTGCCCAGCAAGAAGTTCAAGGTGCTGGGCAACACCGACCGGCACAGCATCAAGAAGA
ACCTGATCGGCGCCCTGCTGTTCGACAGCGGCGAGACCGCCGAGGCCACCCGGCTGAAGCGGACCGCCCGGCGGCGG
TACACCCGGCGGAAGAACCGGATCTGCTACCTGCAGGAGATCTTCAGCAACGAGATGGCCAAGGTGGACGACAGCTT
CTTCCACCGGCTGGAGGAGAGCTTCCTGGTGGAGGAGGACAAGAAGCACGAGCGGCACCCCATCTTCGGCAACATCG
TGGACGAGGTGGCCTACCACGAGAAGTACCCCACCATCTACCACCTGCGGAAGAAGCTGGTGGACAGCACCGACAAG
GCCGACCTGCGGCTGATCTACCTGGCCCTGGCCCACATGATCAAGTTCCGGGGCCACTTCCTGATCGAGGGCGACCT
GAACCCCGACAACAGCGACGTGGACAAGCTGTTCATCCAGCTGGTGCAGACCTACAACCAGCTGTTCGAGGAGAACC
CCATCAACGCCAGCGGCGTGGACGCCAAGGCCATCCTGAGCGCCCGGCTGAGCAAGAGCCGGCGGCTGGAGAACCTG
ATCGCCCAGCTGCCCGGCGAGAAGAAGAACGGCCTGTTCGGCAACCTGATCGCCCTGAGCCTGGGCCTGACCCCCAA
CTTCAAGAGCAACTTCGACCTGGCCGAGGACGCCAAGCTGCAGCTGAGCAAGGACACCTACGACGACGACCTGGACA
ACCTGCTGGCCCAGATCGGCGACCAGTACGCCGACCTGTTCCTGGCCGCCAAGAACCTGAGCGACGCCATCCTGCTG
AGCGACATCCTGCGGGTGAACACCGAGATCACCAAGGCCCCCCTGAGCGCCAGCATGATCAAGCGGTACGACGAGCA
CCACCAGGACCTGACCCTGCTGAAGGCCCTGGTGCGGCAGCAGCTGCCCGAGAAGTACAAGGAGATCTTCTTCGACC
AGAGCAAGAACGGCTACGCCGGCTACATCGACGGCGGCGCCAGCCAGGAGGAGTTCTACAAGTTCATCAAGCCCATC
CTGGAGAAGATGGACGGCACCGAGGAGCTGCTGGTGAAGCTGAACCGGGAGGACCTGCTGCGGAAGCAGCGGACCTT
CGACAACGGCAGCATCCCCCACCAGATCCACCTGGGCGAGCTGCACGCCATCCTGCGGCGGCAGGAGGACTTCTACC
CCTTCCTGAAGGACAACCGGGAGAAGATCGAGAAGATCCTGACCTTCCGGATCCCCTACTACGTGGGCCCCCTGGCC
CGGGGCAACAGCCGGTTCGCCTGGATGACCCGGAAATCCGAGGAGACCATCACCCCCTGGAACTTCGAGGAGGTGGT
GGACAAGGGCGCCAGCGCCCAGAGCTTCATCGAGCGGATGACCAACTTCGACAAGAACCTGCCCAACGAGAAGGTGC
TGCCCAAGCACAGCCTGCTGTACGAGTACTTCACCGTGTACAACGAGCTGACCAAGGTGAAGTACGTGACCGAGGGC
ATGCGGAAGCCCGCCTTCCTGAGCGGCGAGCAGAAGAAGGCCATCGTGGACCTGCTGTTCAAGACCAACCGGAAGGT
GACCGTGAAGCAGCTGAAGGAGGACTACTTCAAGAAGATCGAGTGCTTCGACAGCGTGGAGATCAGCGGCGTGGAGG
ACCGGTTCAACGCCAGCCTGGGCACCTACCACGACCTGCTGAAGATCATCAAGGACAAGGACTTCCTGGACAACGAG
GAGAACGAGGACATCCTGGAGGACATCGTGCTGACCCTGACCCTGTTCGAGGACCGGGAGATGATCGAGGAGCGGCT
GAAAACCTACGCCCACCTGTTCGACGACAAGGTGATGAAGCAGCTGAAGCGGCGGCGGTACACCGGCTGGGGCCGGC
TGAGCCGGAAGCTGATCAACGGCATCCGGGACAAGCAGAGCGGCAAGACCATCCTGGACTTCCTGAAATCCGACGGC
TTCGCCAACCGGAACTTCATGCAGCTGATCCACGACGACAGCCTGACCTTCAAGGAGGACATCCAGAAGGCCCAGGT
GAGCGGCCAGGGCGACAGCCTGCACGAGCACATCGCCAACCTGGCCGGCAGCCCCGCCATCAAGAAGGGCATCCTGC
AGACCGTGAAGGTGGTGGACGAGCTGGTGAAGGTGATGGGCCGGCACAAGCCCGAGAACATCGTGATCGAGATGGCC
CGGGAGAACCAGACCACCCAGAAGGGCCAGAAGAACAGCCGGGAGCGGATGAAGCGGATCGAGGAGGGCATCAAGGA
GCTGGGCAGCCAGATCCTGAAGGAGCACCCCGTGGAGAACACCCAGCTGCAGAACGAGAAGCTGTACCTGTACTACC
TGCAGAACGGCCGGGACATGTACGTGGACCAGGAGCTGGACATCAACCGGCTGAGCGACTACGACGTGGCCGCCATC
GTGCCCCAGAGCTTCCTGAAGGACGACAGCATCGACAACAAGGTGCTGACCCGGAGCGACAAGGCCCGGGGCAAGAG
CGACAACGTGCCCAGCGAGGAGGTGGTGAAGAAGATGAAGAACTACTGGCGGCAGCTGCTGAACGCCAAGCTGATCA
CCCAGCGGAAGTTCGACAACCTGACCAAGGCCGAGCGGGGCGGCCTGAGCGAGCTGGACAAGGCCGGCTTCATCAAG
CGGCAGCTGGTGGAGACCCGGCAGATCACCAAGCACGTGGCCCAGATCCTGGACAGCCGGATGAACACCAAGTACGA
CGAGAACGACAAGCTGATCCGGGAGGTGAAGGTGATCACCCTGAAATCCAAGCTGGTGAGCGACTTCCGGAAGGACT
TCCAGTTCTACAAGGTGCGGGAGATCAACAACTACCACCACGCCCACGACGCCTACCTGAACGCCGTGGTGGGCACC
GCCCTGATCAAGAAGTACCCCAAGCTGGAGAGCGAGTTCGTGTACGGCGACTACAAGGTGTACGACGTGCGGAAGAT
GATCGCCAAGAGCGAGCAGGAGATCGGCAAGGCCACCGCCAAGTACTTCTTCTACAGCAACATCATGAACTTCTTCA
AGACCGAGATCACCCTGGCCAACGGCGAGATCCGGAAGCGGCCCCTGATCGAGACCAACGGCGAGACCGGCGAGATC
GTGTGGGACAAGGGCCGGGACTTCGCCACCGTGCGGAAGGTGCTGAGCATGCCCCAGGTGAACATCGTGAAGAAAAC
CGAGGTGCAGACCGGCGGCTTCAGCAAGGAGAGCATCCTGCCCAAGCGGAACAGCGACAAGCTGATCGCCCGGAAGA
AGGACTGGGACCCCAAGAAGTACGGCGGCTTCGACAGCCCCACCGTGGCCTACAGCGTGCTGGTGGTGGCCAAGGTG
GAGAAGGGCAAGAGCAAGAAGCTGAAATCCGTGAAGGAGCTGCTGGGCATCACCATCATGGAGCGGAGCAGCTTCGA
GAAGAACCCCATCGACTTCCTGGAGGCCAAGGGCTACAAGGAGGTGAAGAAGGACCTGATCATCAAGCTGCCCAAGT
ACAGCCTGTTCGAGCTGGAGAACGGCCGGAAGCGGATGCTGGCCAGCGCCGGCGAGCTGCAGAAGGGCAACGAGCTG
GCCCTGCCCAGCAAGTACGTGAACTTCCTGTACCTGGCCAGCCACTACGAGAAGCTGAAGGGCAGCCCCGAGGACAA
CGAGCAGAAGCAGCTGTTCGTGGAGCAGCACAAGCACTACCTGGACGAGATCATCGAGCAGATCAGCGAGTTCAGCA
AGCGGGTGATCCTGGCCGACGCCAACCTGGACAAGGTGCTGAGCGCCTACAACAAGCACCGGGACAAGCCCATCCGG
GAGCAGGCCGAGAACATCATCCACCTGTTCACCCTGACCAACCTGGGCGCCCCCGCCGCCTTCAAGTACTTCGACAC
CACCATCGACCGGAAGCGGTACACCAGCACCAAGGAGGTGCTGGACGCCACCCTGATCCACCAGAGCATCACCGGCC
TGTACGAGACCCGGATCGACCTGAGCCAGCTGGGCGGCGACAAGCGGCCCGCCGCCACCAAGAAGGCCGGCCAGGCC
AAGAAGAAGAAGGGCCGGGCCGACGCCCTGGACGACTTCGACCTGGACATGCTGGGCAGCGACGCCCTGGACGACTT
CGACCTGGACATGCTGGGCAGCGACGCCCTGGACGACTTCGACCTGGACATGCTGGGCAGCGACGCCCTGGACGACT
TCGACCTGGACATGCTGAGCGGCGGCCCCAAGAAGAAGCGGAAGGTGGGCAGCCAGTACCTGCCCGACACCGACGAC
CGGCACCGGATCGAGGAGAAGCGGAAGCGGACCTACGAGACCTTCAAGAGCATCATGAAGAAATCCCCCTTCAGCGG
CCCCACCGACCCCCGGCCCCCCCCCCGGCGGATCGCCGTGCCCAGCCGGAGCAGCGCCAGCGTGCCCAAGCCCGCCC
CCCAGCCCTACCCCTTCACCAGCAGCCTGAGCACCATCAACTACGACGAGTTCCCCACCATGGTGTTCCCCAGCGGC
CAGATCAGCCAGGCCAGCGCCCTGGCCCCCGCCCCCCCCCAGGTGCTGCCCCAGGCCCCCGCCCCCGCCCCCGCCCC
CGCCATGGTGAGCGCCCTGGCCCAGGCCCCCGCCCCCGTGCCCGTGCTGGCCCCCGGCCCCCCCCAGGCCGTGGCCC
CCCCCGCCCCCAAGCCCACCCAGGCCGGCGAGGGCACCCTGAGCGAGGCCCTGCTGCAGCTGCAGTTCGACGACGAG
GACCTGGGCGCCCTGCTGGGCAACAGCACCGACCCCGCCGTGTTCACCGACCTGGCCAGCGTGGACAACAGCGAGTT
CCAGCAGCTGCTGAACCAGGGCATCCCCGTGGCCCCCCACACCACCGAGCCCATGCTGATGGAGTACCCCGAGGCCA
TCACCCGGCTGGTGACCGGCGCCCAGCGGCCCCCCGACCCCGCCCCCGCCCCCCTGGGCGCCCCCGGCCTGCCCAAC
GGCCTGCTGAGCGGCGACGAGGACTTCAGCAGCATCGCCGACATGGACTTCAGCGCCCTGCTGGGCAGCGGCAGCGG
CAGCCGGGACAGCCGGGAGGGCATGTTCCTGCCCAAGCCCGAGGCCGGCAGCGCCATCAGCGACGTGTTCGAGGGCC
GGGAGGTGTGCCAGCCCAAGCGGATCCGGCCCTTCCACCCCCCCGGCAGCCCCTGGGCCAACCGGCCCCTGCCCGCC
AGCCTGGCCCCCACCCCCACCGGCCCCGTGCACGAGCCCGTGGGCAGCCTGACCCCCGCCCCCGTGCCCCAGCCCCT
GGACCCCGCCCCCGCCGTGACCCCCGAGGCCAGCCACCTGCTGGAGGACCCCGACGAGGAGACCAGCCAGGCCGTGA
AGGCCCTGCGGGAGATGGCCGACACCGTGATCCCCCAGAAGGAGGAGGCCGCCATCTGCGGCCAGATGGACCTGAGC
CACCCCCCCCCCCGGGGCCACCTGGACGAGCTGACCACCACCCTGGAGAGCATGACCGAGGACCTGAACCTGGACAG
CCCCCTGACCCCCGAGCTGAACGAGATCCTGGACACCTTCCTGAACGACGAGTGCCTGCTGCACGCCATGCACATCA
GCACCGGCCTGAGCATCTTCGACACCAGCCTGTTCAGCGGCGGCAAGCGGCCCGCCGCCACCAAGAAGGCCGGCCAG
GCCAAGAAGAAGAAGGGCAGCTACCCCTACGACGTGCCCGACTACGCCTGAGCGGCCGCTTAATTAAGCTGCCTTCT
GCGGGGCTTGCCTTCTGGCCATGCCCTTCTTCTCTCCCTTGCACCTGTACCTCTTGGTCTTTGAATAAAGCCTGAGT
AGGAAGTCTAGAAAAAAAAAAAAAAAAAAAAAAAAAAAAAAAAAAAAAAAAAAAAAAAAAAAAAAAAAAAAAAAAAA
AAAAAAAAAAAAAA
dCas9-VPR (VP64-p65-RTA) exemplary sequence 2
(SEO ID NO: 309)
ATGGCCCCCAAGAAGAAGCGGAAGGTGGGCATCCACGGCGTGCCCGCCGCCGACAAGAAGTACAGCATCGGCCTGGC
CATCGGCACCAACAGCGTGGGCTGGGCCGTGATCACCGACGAGTACAAGGTGCCCAGCAAGAAGTTCAAGGTGCTGG
GCAACACCGACCGGCACAGCATCAAGAAGAACCTGATCGGCGCCCTGCTGTTCGACAGCGGCGAGACCGCCGAGGCC
ACCCGGCTGAAGCGGACCGCCCGGCGGCGGTACACCCGGCGGAAGAACCGGATCTGCTACCTGCAGGAGATCTTCAG
CAACGAGATGGCCAAGGTGGACGACAGCTTCTTCCACCGGCTGGAGGAGAGCTTCCTGGTGGAGGAGGACAAGAAGC
ACGAGCGGCACCCCATCTTCGGCAACATCGTGGACGAGGTGGCCTACCACGAGAAGTACCCCACCATCTACCACCTG
CGGAAGAAGCTGGTGGACAGCACCGACAAGGCCGACCTGCGGCTGATCTACCTGGCCCTGGCCCACATGATCAAGTT
CCGGGGCCACTTCCTGATCGAGGGCGACCTGAACCCCGACAACAGCGACGTGGACAAGCTGTTCATCCAGCTGGTGC
AGACCTACAACCAGCTGTTCGAGGAGAACCCCATCAACGCCAGCGGCGTGGACGCCAAGGCCATCCTGAGCGCCCGG
CTGAGCAAGAGCCGGCGGCTGGAGAACCTGATCGCCCAGCTGCCCGGCGAGAAGAAGAACGGCCTGTTCGGCAACCT
GATCGCCCTGAGCCTGGGCCTGACCCCCAACTTCAAGAGCAACTTCGACCTGGCCGAGGACGCCAAGCTGCAGCTGA
GCAAGGACACCTACGACGACGACCTGGACAACCTGCTGGCCCAGATCGGCGACCAGTACGCCGACCTGTTCCTGGCC
GCCAAGAACCTGAGCGACGCCATCCTGCTGAGCGACATCCTGCGGGTGAACACCGAGATCACCAAGGCCCCCCTGAG
CGCCAGCATGATCAAGCGGTACGACGAGCACCACCAGGACCTGACCCTGCTGAAGGCCCTGGTGCGGCAGCAGCTGC
CCGAGAAGTACAAGGAGATCTTCTTCGACCAGAGCAAGAACGGCTACGCCGGCTACATCGACGGCGGCGCCAGCCAG
GAGGAGTTCTACAAGTTCATCAAGCCCATCCTGGAGAAGATGGACGGCACCGAGGAGCTGCTGGTGAAGCTGAACCG
GGAGGACCTGCTGCGGAAGCAGCGGACCTTCGACAACGGCAGCATCCCCCACCAGATCCACCTGGGCGAGCTGCACG
CCATCCTGCGGCGGCAGGAGGACTTCTACCCCTTCCTGAAGGACAACCGGGAGAAGATCGAGAAGATCCTGACCTTC
CGGATCCCCTACTACGTGGGCCCCCTGGCCCGGGGCAACAGCCGGTTCGCCTGGATGACCCGGaaatccGAGGAGAC
CATCACCCCCTGGAACTTCGAGGAGGTGGTGGACAAGGGCGCCAGCGCCCAGAGCTTCATCGAGCGGATGACCAACT
TCGACAAGAACCTGCCCAACGAGAAGGTGCTGCCCAAGCACAGCCTGCTGTACGAGTACTTCACCGTGTACAACGAG
CTGACCAAGGTGAAGTACGTGACCGAGGGCATGCGGAAGCCCGCCTTCCTGAGCGGCGAGCAGAAGAAGGCCATCGT
GGACCTGCTGTTCAAGACCAACCGGAAGGTGACCGTGAAGCAGCTGAAGGAGGACTACTTCAAGAAGATCGAGTGCT
TCGACAGCGTGGAGATCAGCGGCGTGGAGGACCGGTTCAACGCCAGCCTGGGCACCTACCACGACCTGCTGAAGATC
ATCAAGGACAAGGACTTCCTGGACAACGAGGAGAACGAGGACATCCTGGAGGACATCGTGCTGACCCTGACCCTGTT
CGAGGACCGGGAGATGATCGAGGAGCGGCTGAAaACCTACGCCCACCTGTTCGACGACAAGGTGATGAAGCAGCTGA
AGCGGCGGCGGTACACCGGCTGGGGCCGGCTGAGCCGGAAGCTGATCAACGGCATCCGGGACAAGCAGAGCGGCAAG
ACCATCCTGGACTTCCTGaaatccGACGGCTTCGCCAACCGGAACTTCATGCAGCTGATCCACGACGACAGCCTGAC
CTTCAAGGAGGACATCCAGAAGGCCCAGGTGAGCGGCCAGGGCGACAGCCTGCACGAGCACATCGCCAACCTGGCCG
GCAGCCCCGCCATCAAGAAGGGCATCCTGCAGACCGTGAAGGTGGTGGACGAGCTGGTGAAGGTGATGGGCCGGCAC
AAGCCCGAGAACATCGTGATCGAGATGGCCCGGGAGAACCAGACCACCCAGAAGGGCCAGAAGAACAGCCGGGAGCG
GATGAAGCGGATCGAGGAGGGCATCAAGGAGCTGGGCAGCCAGATCCTGAAGGAGCACCCCGTGGAGAACACCCAGC
TGCAGAACGAGAAGCTGTACCTGTACTACCTGCAGAACGGCCGGGACATGTACGTGGACCAGGAGCTGGACATCAAC
CGGCTGAGCGACTACGACGTGGCCGCCATCGTGCCCCAGAGCTTCCTGAAGGACGACAGCATCGACAACAAGGTGCT
GACCCGGAGCGACAAGGCCCGGGGCAAGAGCGACAACGTGCCCAGCGAGGAGGTGGTGAAGAAGATGAAGAACTACT
GGCGGCAGCTGCTGAACGCCAAGCTGATCACCCAGCGGAAGTTCGACAACCTGACCAAGGCCGAGCGGGGCGGCCTG
AGCGAGCTGGACAAGGCCGGCTTCATCAAGCGGCAGCTGGTGGAGACCCGGCAGATCACCAAGCACGTGGCCCAGAT
CCTGGACAGCCGGATGAACACCAAGTACGACGAGAACGACAAGCTGATCCGGGAGGTGAAGGTGATCACCCTGaaat
ccAAGCTGGTGAGCGACTTCCGGAAGGACTTCCAGTTCTACAAGGTGCGGGAGATCAACAACTACCACCACGCCCAC
GACGCCTACCTGAACGCCGTGGTGGGCACCGCCCTGATCAAGAAGTACCCCAAGCTGGAGAGCGAGTTCGTGTACGG
CGACTACAAGGTGTACGACGTGCGGAAGATGATCGCCAAGAGCGAGCAGGAGATCGGCAAGGCCACCGCCAAGTACT
TCTTCTACAGCAACATCATGAACTTCTTCAAGACCGAGATCACCCTGGCCAACGGCGAGATCCGGAAGCGGCCCCTG
ATCGAGACCAACGGCGAGACCGGCGAGATCGTGTGGGACAAGGGCCGGGACTTCGCCACCGTGCGGAAGGTGCTGAG
CATGCCCCAGGTGAACATCGTGAAGAAaACCGAGGTGCAGACCGGCGGCTTCAGCAAGGAGAGCATCCTGCCCAAGC
GGAACAGCGACAAGCTGATCGCCCGGAAGAAGGACTGGGACCCCAAGAAGTACGGCGGCTTCGACAGCCCCACCGTG
GCCTACAGCGTGCTGGTGGTGGCCAAGGTGGAGAAGGGCAAGAGCAAGAAGCTGaaatccGTGAAGGAGCTGCTGGG
CATCACCATCATGGAGCGGAGCAGCTTCGAGAAGAACCCCATCGACTTCCTGGAGGCCAAGGGCTACAAGGAGGTGA
AGAAGGACCTGATCATCAAGCTGCCCAAGTACAGCCTGTTCGAGCTGGAGAACGGCCGGAAGCGGATGCTGGCCAGC
GCCGGCGAGCTGCAGAAGGGCAACGAGCTGGCCCTGCCCAGCAAGTACGTGAACTTCCTGTACCTGGCCAGCCACTA
CGAGAAGCTGAAGGGCAGCCCCGAGGACAACGAGCAGAAGCAGCTGTTCGTGGAGCAGCACAAGCACTACCTGGACG
AGATCATCGAGCAGATCAGCGAGTTCAGCAAGCGGGTGATCCTGGCCGACGCCAACCTGGACAAGGTGCTGAGCGCC
TACAACAAGCACCGGGACAAGCCCATCCGGGAGCAGGCCGAGAACATCATCCACCTGTTCACCCTGACCAACCTGGG
CGCCCCCGCCGCCTTCAAGTACTTCGACACCACCATCGACCGGAAGCGGTACACCAGCACCAAGGAGGTGCTGGACG
CCACCCTGATCCACCAGAGCATCACCGGCCTGTACGAGACCCGGATCGACCTGAGCCAGCTGGGCGGCGACAAGCGG
CCCGCCGCCACCAAGAAGGCCGGCCAGGCCAAGAAGAAGAAGGGCCGGGCCGACGCCCTGGACGACTTCGACCTGGA
CATGCTGGGCAGCGACGCCCTGGACGACTTCGACCTGGACATGCTGGGCAGCGACGCCCTGGACGACTTCGACCTGG
ACATGCTGGGCAGCGACGCCCTGGACGACTTCGACCTGGACATGCTGAGCGGCGGCCCCAAGAAGAAGCGGAAGGTG
GGCAGCCAGTACCTGCCCGACACCGACGACCGGCACCGGATCGAGGAGAAGCGGAAGCGGACCTACGAGACCTTCAA
GAGCATCATGAAGaaatccCCCTTCAGCGGCCCCACCGACCCCCGGCCCCCCCCCCGGCGGATCGCCGTGCCCAGCC
GGAGCAGCGCCAGCGTGCCCAAGCCCGCCCCCCAGCCCTACCCCTTCACCAGCAGCCTGAGCACCATCAACTACGAC
GAGTTCCCCACCATGGTGTTCCCCAGCGGCCAGATCAGCCAGGCCAGCGCCCTGGCCCCCGCCCCCCCCCAGGTGCT
GCCCCAGGCCCCCGCCCCCGCCCCCGCCCCCGCCATGGTGAGCGCCCTGGCCCAGGCCCCCGCCCCCGTGCCCGTGC
TGGCCCCCGGCCCCCCCCAGGCCGTGGCCCCCCCCGCCCCCAAGCCCACCCAGGCCGGCGAGGGCACCCTGAGCGAG
GCCCTGCTGCAGCTGCAGTTCGACGACGAGGACCTGGGCGCCCTGCTGGGCAACAGCACCGACCCCGCCGTGTTCAC
CGACCTGGCCAGCGTGGACAACAGCGAGTTCCAGCAGCTGCTGAACCAGGGCATCCCCGTGGCCCCCCACACCACCG
AGCCCATGCTGATGGAGTACCCCGAGGCCATCACCCGGCTGGTGACCGGCGCCCAGCGGCCCCCCGACCCCGCCCCC
GCCCCCCTGGGCGCCCCCGGCCTGCCCAACGGCCTGCTGAGCGGCGACGAGGACTTCAGCAGCATCGCCGACATGGA
CTTCAGCGCCCTGCTGGGCAGCGGCAGCGGCAGCCGGGACAGCCGGGAGGGCATGTTCCTGCCCAAGCCCGAGGCCG
GCAGCGCCATCAGCGACGTGTTCGAGGGCCGGGAGGTGTGCCAGCCCAAGCGGATCCGGCCCTTCCACCCCCCCGGC
AGCCCCTGGGCCAACCGGCCCCTGCCCGCCAGCCTGGCCCCCACCCCCACCGGCCCCGTGCACGAGCCCGTGGGCAG
CCTGACCCCCGCCCCCGTGCCCCAGCCCCTGGACCCCGCCCCCGCCGTGACCCCCGAGGCCAGCCACCTGCTGGAGG
ACCCCGACGAGGAGACCAGCCAGGCCGTGAAGGCCCTGCGGGAGATGGCCGACACCGTGATCCCCCAGAAGGAGGAG
GCCGCCATCTGCGGCCAGATGGACCTGAGCCACCCCCCCCCCCGGGGCCACCTGGACGAGCTGACCACCACCCTGGA
GAGCATGACCGAGGACCTGAACCTGGACAGCCCCCTGACCCCCGAGCTGAACGAGATCCTGGACACCTTCCTGAACG
ACGAGTGCCTGCTGCACGCCATGCACATCAGCACCGGCCTGAGCATCTTCGACACCAGCCTGTTCAGCGGCGGCAAG
CGGCCCGCCGCCACCAAGAAGGCCGGCCAGGCCAAGAAGAAGAAGGGCAGCTACCCCTACGACGTGCCCGACTACGC
CTGA
(SEQ ID NO: 305)
MAPKKKRKVGIHGVPAADKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFLKDNREKIEKILTF
RIPYYVGPLARGNSRFAWMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRIDLSQLGGDKR
PAATKKAGQAKKKKGRADALDDFDLDMLGSDALDDFDLDMLGSDALDDFDLDMLGSDALDDFDLDMLSGGPKKKRKV
GSQYLPDTDDRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPTQAGEGTLSE
ALLQLQFDDEDLGALLGNSTDPAVFTDLASVDNSEFQQLLNQGIPVAPHTTEPMLMEYPEAITRLVTGAQRPPDPAP
APLGAPGLPNGLLSGDEDFSSIADMDFSALLGSGSGSRDSREGMFLPKPEAGSAISDVFEGREVCQPKRIRPFHPPG
SPWANRPLPASLAPTPTGPVHEPVGSLTPAPVPQPLDPAPAVTPEASHLLEDPDEETSQAVKALREMADTVIPQKEE
AAICGQMDLSHPPPRGHLDELTTTLESMTEDLNLDSPLTPELNEILDTFLNDECLLHAMHISTGLSIFDTSLFSGGK
RPAATKKAGQAKKKKGSYPYDVPDYA

In some embodiments, a modulating agent comprises a targeting moiety comprising a Zn finger molecule and an effector moiety comprising a transcription activator activity, e.g., VP64 or a functional fragment or variant thereof. In some embodiments, a modulating agent comprises a targeting moiety comprising a Zn finger molecule comprising 6, 7, 8, 9, or 10 Zn finger proteins (e.g., 9) and an effector moiety comprising VP64 or a functional fragment or variant thereof.

In some embodiments, a modulating agent is encoded by the nucleic acid sequence of SEQ ID NO: 302 or a sequence with at least 80, 85, 90, 95, 96, 97, 98, or 99% identity to said sequence. In some embodiments, a modulating agent comprises an amino acid sequence of SEQ ID NO: 306 or an amino acid sequence encoded by the nucleic acid sequence of SEQ ID NO: 302, or an amino acid sequence with at least 80, 85, 90, 95, 96, 97, 98, or 99% identity to either of the same.

ZF9-VPR
(SEQ ID NO: 302)
AGGAAAUAAGAGAGAAAAGAAGAGUAAGAAGAAAUAUAAGAGCCACCAU
GGCCCCCAAGAAGAAGCGGAAGGUGGGCAUCCACGGCGUGCCCGCCGCC
CUGGAACCGGGCGAAAAACCAUACAAAUGUCCGGAAUGUGGCAAGAGCU
UUAGUCGCGCCGAUAAUCUGACGGAACACCAGCGCACCCACACCCAUCC
ACGUGCCCCGAUCCCGAAACCGUUCCAAUAUAAAUGCCCAGAAUGCGGU
AAGAGCUUCAGCGACAAGAAGGAUCUGACCCGCCACCAACGCACGCACA
CCGGCGAAAAGCCGUACAAAUGUCCAGAGUGCGGCAAAAGUUUCAGCCG
CGAGGAUAACCUCCAUACGCAUCAGCGUACCCACACCGGUGAAAAGCCA
UACAAGUGUCCGGAAUGCGGCAAAAGCUUUAGCACGAGCGGUCAUCUGG
UGCGUCACCAGCGUACCCAUACGGGUGAGAAACCAUAUAAGUGUCCAGA
GUGUGGUAAGAGCUUUAGCCAGCUGGCGCAUCUGCGUGCCCAUCAACGU
ACGCAUACCGGUGAGAAACCAUACAAGUGCCCGGAGUGUGGUAAAAGCU
UCAGCACGAGCGGCGAACUGGUUCGCCAUCAGCGCACGCACACGGGCGA
AAAGCCAUAUAAGUGCCCGGAAUGUGGUAAGAGUUUUAGUCAGAGCGGU
GAUCUGCGUCGUCACCAACGUACCCAUACCGGCGAGAAGCCAACCGGCA
AGAAAACCAGCUCGGGCGGGGGUGGCUCAGAUGCUCUCGAUGAUUUCGA
UCUCGACAUGCUCGGCUCCGACGCUCUGGACGAUUUUGAUCUGGACAUG
CUCGGCAGCGACGCCCUCGACGAUUUCGAUCUGGAUAUGCUGGGCUCCG
ACGCCCUCGACGACUUUGAUCUCGACAUGCUCGGCAGCGGCUCCGGCAG
CCAAUACCUCCCCGACACCGACGAUAGACAUAGAAUUGAGGAAAAGAGG
AAAAGGACAUAUGAAACCUUUAAGUCCAUCAUGAAGAAGUCCCCCUUUA
GCGGACCUACAGACCCUAGGCCUCCUCCCAGAAGGAUCGCCGUCCCUUC
CAGAUCCAGCGCUUCCGUGCCUAAGCCCGCCCCCCAACCCUACCCCUUC
ACAUCCUCCCUCAGCACCAUCAACUACGACGAGUUUCCCACAAUGGUGU
UCCCCUCCGGCCAGAUUAGCCAAGCCUCCGCUCUCGCCCCCGCCCCCCC
UCAAGUCCUCCCUCAAGCCCCCGCCCCCGCCCCCGCCCCCGCUAUGGUG
AGCGCUCUGGCCCAAGCCCCCGCCCCCGUGCCCGUGCUCGCUCCCGGAC
CUCCUCAAGCCGUGGCUCCCCCCGCCCCCAAACCUACACAAGCUGGCGA
GGGCACACUGUCCGAAGCUCUGCUGCAGCUGCAAUUCGAUGAUGAGGAU
CUGGGAGCUCUGCUGGGAAACUCCACCGAUCCCGCCGUGUUCACCGAUC
UGGCCAGCGUCGACAAUAGCGAGUUCCAACAGCUGCUGAAUCAAGGAAU
CCCCGUGGCUCCUCAUACCACCGAGCCUAUGCUCAUGGAAUACCCCGAG
GCUAUCACAAGACUGGUGACCGGAGCUCAAAGACCCCCCGACCCCGCCC
CCGCUCCUCUGGGCGCCCCCGGACUGCCUAAUGGACUGCUGAGCGGCGA
CGAGGAUUUCUCCUCCAUUGCCGAUAUGGACUUUUCCGCCCUCCUCGGC
AGCGGCAGCGGAAGCAGAGAUUCUAGAGAAGGCAUGUUUCUGCCCAAAC
CCGAAGCUGGCUCCGCUAUCUCCGACGUCUUUGAGGGAAGAGAGGUGUG
UCAGCCUAAGAGAAUCAGACCCUUUCACCCUCCCGGCAGCCCUUGGGCU
AAUAGACCUCUCCCCGCCAGCCUCGCCCCUACACCUACCGGCCCCGUGC
AUGAGCCCGUCGGAAGCCUCACACCCGCCCCCGUGCCUCAACCUCUCGA
CCCCGCCCCCGCCGUCACCCCCGAAGCUUCCCAUCUCCUCGAAGAUCCC
GACGAAGAAACAUCCCAAGCCGUGAAAGCCCUCAGAGAAAUGGCUGACA
CCGUGAUCCCUCAAAAGGAGGAAGCCGCUAUCUGUGGCCAAAUGGAUCU
GAGCCACCCUCCCCCUAGAGGACAUCUCGAUGAGCUCACAACCACCCUC
GAAAGCAUGACCGAAGAUCUCAAUCUGGAUUCCCCUCUGACACCCGAAC
UCAACGAGAUUCUCGACACCUUUCUGAACGACGAGUGUCUGCUGCACGC
CAUGCACAUCUCCACCGGCCUCAGCAUCUUCGAUACCAGCCUCUUUGGC
AGCGGCUCCGGCAGCGGAGGUGGCGGAUCGGGAAAGCGGCCCGCCGCCA
CCAAGAAGGCCGGCCAGGCCAAGAAGAAGAAGGGCAGCUACCCCUACGA
CGUGCCCGACUACGCCUGAGCGGCCGCUUAAUUAAGCUGCCUUCUGCGG
GGCUUGCCUUCUGGCCAUGCCCUUCUUCUCUCCCUUGCACCUGUACCUC
UUGGUCUUUGAAUAAAGCCUGAGUAGGAAGUCUAGAAAAAAAAAAAAAA
AAAAAAAAAAAAAAAAAAAAAAAAAAAAAAAAAAAAAAAAAAAAAAAAA
AAAAAAAAAAAAAAAAA
(SEQ ID NO: 306)
MAPKKKRKVGIHGVPAALEPGEKPYKCPECGKSFSRADNLTEHQRTHTH
PRAPIPKPFQYKCPECGKSFSDKKDLTRHQRTHTGEKPYKCPECGKSFS
REDNLHTHQRTHTGEKPYKCPECGKSFSTSGHLVRHQRTHTGEKPYKCP
ECGKSFSQLAHLRAHQRTHTGEKPYKCPECGKSFSTSGELVRHQRTHTG
EKPYKCPECGKSFSQSGDLRRHQRTHTGEKPTGKKTSSGGGGSDALDDF
DLDMLGSDALDDFDLDMLGSDALDDFDLDMLGSDALDDFDLDMLGSGSG
SQYLPDTDDRHRIEEKRKRTYETFKSIMKKSPFSGPTDPRPPPRRIAVP
SRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAG
EGTLSEALLQLQFDDEDLGALLGNSTDPAVFTDLASVDNSEFQQLLNQG
IPVAPHTTEPMLMEYPEAITRLVTGAQRPPDPAPAPLGAPGLPNGLLSG
DEDFSSIADMDFSALLGSGSGSRDSREGMFLPKPEAGSAISDVFEGREV
CQPKRIRPFHPPGSPWANRPLPASLAPTPTGPVHEPVGSLTPAPVPQPL
DPAPAVTPEASHLLEDPDEETSQAVKALREMADTVIPQKEEAAICGQMD
LSHPPPRGHLDELTTTLESMTEDLNLDSPLTPELNEILDTFLNDECLLH
AMHISTGLSIFDTSLFGSGSGSGGGGSGKRPAATKKAGQAKKKKGSYPY
DVPDYA

In some embodiments, a modulating agent comprises a targeting moiety comprising a TAL effector molecule and an effector moiety comprising a transcription activator activity, e.g., VP64 or a functional fragment or variant thereof.

In some embodiments, a modulating agent is encoded by the nucleic acid sequence of SEQ ID NO: 303 or a sequence with at least 80, 85, 90, 95, 96, 97, 98, or 99% identity to said sequence. In some embodiments, a modulating agent comprises an amino acid sequence of SEQ ID NO: 307, an amino acid sequence encoded by the nucleic acid sequence of SEQ ID NO: 303, or an amino acid sequence with at least 80, 85, 90, 95, 96, 97, 98, or 99% identity to either of the same.

TAL-VPR
(SEQ ID NO: 303)
AGGAAAUAAGAGAGAAAAGAAGAGUAAGAAGAAAUAUAAGAGCCACCAUGGCCCCCAAGAAGAAGCGGAAGGUGGGCA
UCCACGGCGUGCCCGCCGCCGGCAGCAGCGGAUCCCAUAUGGUUGAUCUGCGUACCCUGGGUUAUAGCCAGCAGCAGC
AAGAAAAAAUCAAACCGAAAGUUCGUAGCACCGUUGCACAGCAUCAUGAAGCACUGGUUGGUCAUGGUUUUACCCAUG
CACAUAUUGUUGCACUGAGCCAGCAUCCGGCAGCACUGGGCACCGUUGCAGUUAAAUAUCAGGAUAUGAUUGCAGCAC
UGCCGGAAGCAACCCAUGAAGCAAUUGUUGGUGUUGGUAAACGCGGAGCUGGUGCACGUGCCCUGGAAGCACUGCUGA
CCGUUGCCGGUGAACUGCGUGGUCCGCCUCUGCAGCUGGAUACCGGUCAGCUGCUGAAAAUUGCAAAACGUGGUGGUG
UUACCGCAGUUGAAGCAGUUCAUGCAUGGCGUAAUGCACUGACCGGUGCACCGCUGAAUCUGACACCGGAACAGGUUG
UUGCAAUUGCCAGCCAUGAUGGUGGCAAACAGGCACUGGAAACCGUUCAGCGUCUGCUGCCGGUUCUGUGUCAGGCAC
AUGGUCUGACCCCUGAACAGGUGGUGGCCAUUGCAAGCCAUGACGGCGGUAAACAAGCCCUGGAAACAGUGCAGCGCC
UGUUACCGGUGCUGUGCCAGGCCCAUGGCUUAACUCCGGAACAGGUGGUAGCGAUCGCAUCAAAUGGUGGAGGUAAAC
AGGCCUUAGAAACCGUACAGCGCUUACUGCCGGUGUUAUGCCAGGCGCACGGCCUGACGCCAGAACAGGUAGUGGCAA
UCGCCUCAAAUAAUGGUGGAAAACAGGCGUUAGAGACAGUCCAGCGCCUGCUGCCUGUAUUAUGUCAAGCCCAUGGCC
UGACCCCAGAGCAAGUUGUUGCGAUUGCAAGUAACAUUGGGGGUAAACAGGCACUUGAGACAGUUCAACGUUUACUGC
CUGUACUGUGCCAAGCUCACGGUCUGACUCCGGAACAAGUCGUCGCGAUUGCGAGUAACAAUGGUGGCAAACAAGCAU
UAGAAACGGUGCAACGCCUGCUGCCAGUUCUUUGCCAGGCUCACGGUUUAACCCCUGAGCAGGUUGUAGCUAUUGCGA
GUAAUAAUGGUGGUAAGCAGGCGUUGGAAACUGUGCAAAGACUGCUGCCCGUGUUGUGCCAAGCACAUGGUUUAACCC
CAGAACAAGUCGUAGCAAUCGCAAGCAAUGGCGGUGGCAAGCAAGCGCUUGAAACAGUACAGCGUUUAUUACCGGUAC
UUUGUCAGGCCCACGGUCUUACACCAGAACAAGUUGUGGCCAUAGCCAGUCAUGAUGGCGGAAAGCAGGCUCUGGAAA
CGGUACAACGUCUGUUACCUGUUCUGUGUCAAGCGCACGGAUUAACACCUGAACAAGUAGUUGCCAUUGCGUCAAAUG
GGGGAGGCAAGCAGGCCUUGGAGACAGUGCAGAGAUUACUGCCAGUGUUGUGUCAGGCUCAUGGCCUUACACCCGAGC
AGGUCGUGGCAAUUGCAUCUAAUAUCGGCGGUAAGCAAGCUUUAGAGACUGUUCAGAGACUGCUUCCUGUCCUGUGCC
AGGCACACGGACUUACGCCUGAGCAAGUGGUUGCAAUCGCCUCUAAUAUAGGUGGUAAGCAAGCACUGGAAACUGUCC
AACGCUUACUUCCGGUGCUUUGUCAAGCACACGGCUUAACGCCAGAGCAGGUCGUCGCCAUAGCCAGCCAUGACGGUG
GUAAACAGGCCCUUGAAACGGUCCAAAGACUUCUGCCGGUCCUUUGCCAAGCGCAUGGGCUGACACCUGAGCAGGUAG
UCGCGAUUGCCUCACAUGAUGGUGGGAAGCAGGCAUUAGAAACAGUUCAAAGAUUAUUACCAGUCCUGUGUCAGGCGC
AUGGGUUAACCCCAGAGCAGGUAGUUGCAAUAGCAUCCAAUGGUGGCGGAAAACAAGCGUUGGAAACGGUUCAGCGGU
UAUUGCCUGUUUUGUGCCAGGCGCAUGGUUUGACACCCGAGCAAGUGGUAGCCAUAGCCUCACAUGACGGGGGUAAAC
AAGCUUUGGAGACAGUACAACGGCUGCUUCCAGUUUUAUGUCAGGCCCAUGGAUUGACGCCUGAACAAGUUGUCGCUA
UCGCAAGUAAUGGCGGUGGUAAACAAGCGCUUGAAACCGUUCAACGCCUUCUGCCUGUGCUUUGUCAGGCACAUGGAU
UAACACCCGAACAGGUUGUCGCGAUAGCUUCAAACAUUGGUGGUCGUCCGGCACUGGAAAGCAUUGUUGCACAGCUGA
GCCGUCCUGAUCCGGCACUGGCAGCACUGACCAAUGAUCAUCUGGUUGCACUGGCAUGUCUGGGUGGUCGCCCUGCCC
UGGAUGCAGUUAAAAAAGGUCUGCCGCAUGCACCGGCACUGAUUAAACGUACCAAUCGUCGUAUUCCGGAACGUACCA
GCCAUCGUGUUGCUAGCGGCAGCGGCGGCGGCAGCGGCGGCGACGCCCUGGACGACUUCGACCUGGACAUGCUGGGCA
GCGACGCCCUGGACGACUUCGACCUGGACAUGCUGGGCAGCGACGCCCUGGACGACUUCGACCUGGACAUGCUGGGCA
GCGACGCCCUGGACGACUUCGACCUGGACAUGCUGAGCGGCGGCCCCAAGAAGAAGCGGAAGGUGGGCAGCCAGUACC
UGCCCGACACCGACGACCGGCACCGGAUCGAGGAGAAGCGGAAGCGGACCUACGAGACCUUCAAGAGCAUCAUGAAGA
AAUCCCCCUUCAGCGGCCCCACCGACCCCCGGCCCCCCCCCCGGCGGAUCGCCGUGCCCAGCCGGAGCAGCGCCAGCG
UGCCCAAGCCCGCCCCCCAGCCCUACCCCUUCACCAGCAGCCUGAGCACCAUCAACUACGACGAGUUCCCCACCAUGG
UGUUCCCCAGCGGCCAGAUCAGCCAGGCCAGCGCCCUGGCCCCCGCCCCCCCCCAGGUGCUGCCCCAGGCCCCCGCCC
CCGCCCCCGCCCCCGCCAUGGUGAGCGCCCUGGCCCAGGCCCCCGCCCCCGUGCCCGUGCUGGCCCCCGGCCCCCCCC
AGGCCGUGGCCCCCCCCGCCCCCAAGCCCACCCAGGCCGGCGAGGGCACCCUGAGCGAGGCCCUGCUGCAGCUGCAGU
UCGACGACGAGGACCUGGGCGCCCUGCUGGGCAACAGCACCGACCCCGCCGUGUUCACCGACCUGGCCAGCGUGGACA
ACAGCGAGUUCCAGCAGCUGCUGAACCAGGGCAUCCCCGUGGCCCCCCACACCACCGAGCCCAUGCUGAUGGAGUACC
CCGAGGCCAUCACCCGGCUGGUGACCGGCGCCCAGCGGCCCCCCGACCCCGCCCCCGCCCCCCUGGGCGCCCCCGGCC
UGCCCAACGGCCUGCUGAGCGGCGACGAGGACUUCAGCAGCAUCGCCGACAUGGACUUCAGCGCCCUGCUGGGCAGCG
GCAGCGGCAGCCGGGACAGCCGGGAGGGCAUGUUCCUGCCCAAGCCCGAGGCCGGCAGCGCCAUCAGCGACGUGUUCG
AGGGCCGGGAGGUGUGCCAGCCCAAGCGGCUCCGGCCCUUCCACCCCCCCGGCAGCCCCUGGGCCAACCGGCCCCUGC
CCGCCAGCCUGGCCCCCACCCCCACCGGCCCCGUGCACGAGCCCGUGGGCAGCCUGACCCCCGCCCCCGUGCCCCAGC
CCCUGGACCCCGCCCCCGCCGUGACCCCCGAGGCCAGCCACCUGCUGGAGGACCCCGACGAGGAGACCAGCCAGGCCG
UGAAGGCCCUGCGGGAGAUGGCCGACACCGUGAUCCCCCAGAAGGAGGAGGCCGCCAUCUGCGGCCAGAUGGACCUGA
GCCACCCCCCCCCCCGGGGCCACCUGGACGAGCUGACCACCACCCUGGAGAGCAUGACCGAGGACCUGAACCUGGACA
GCCCCCUGACCCCCGAGCUGAACGAGAUCCUGGACACCUUCCUGAACGACGAGUGCCUGCUGCACGCCAUGCACAUCA
GCACCGGCCUGAGCAUCUUCGACACCAGCCUGUUCAGCGGCGGCAAGCGGCCCGCCGCCACCAAGAAGGCCGGCCAGG
CCAAGAAGAAGAAGGGCAGCUACCCCUACGACGUGCCCGACUACGCCUGAGCGGCCGCUUAAUUAAGCUGCCUUCUGC
GGGGCUUGCCUUCUGGCCAUGCCCUUCUUCUCUCCCUUGCACCUGUACCUCUUGGUCUUUGAAUAAAGCCUGAGUAGG
AAGUCUAGAAAAAAAAAAAAAAAAAAAAAAAAAAAAAAAAAAAAAAAAAAAAAAAAAAAAAAAAAAAAAAAAAAAAAA
AAAAAAAAAA
(SEQ ID NO: 307)
MAPKKKRKVGIHGVPAAGSSGSHMVDLRTLGYSQQQQEKIKPKVRSTVAQHHEALVGHGFTHAHIVALSQHPAALGTV
AVKYQDMIAALPEATHEAIVGVGKRGAGARALEALLTVAGELRGPPLQLDTGQLLKIAKRGGVTAVEAVHAWRNALTG
APLNLTPEQVVAIASHDGGKQALETVQRLLPVLCQAHGLTPEQVVAIASHDGGKQALETVQRLLPVLCQAHGLTPEQV
VAIASNGGGKQALETVQRLLPVLCQAHGLTPEQVVAIASNNGGKQALETVQRLLPVLCQAHGLTPEQVVAIASNIGGK
QALETVQRLLPVLCQAHGLTPEQVVAIASNNGGKQALETVQRLLPVLCQAHGLTPEQVVAIASNNGGKQALETVQRLL
PVLCQAHGLTPEQVVAIASNGGGKQALETVQRLLPVLCQAHGLTPEQVVAIASHDGGKQALETVQRLLPVLCQAHGLT
PEQVVAIASNGGGKQALETVQRLLPVLCQAHGLTPEQVVAIASNIGGKQALETVQRLLPVLCQAHGLTPEQVVAIASN
IGGKQALETVQRLLPVLCQAHGLTPEQVVAIASHDGGKQALETVQRLLPVLCQAHGLTPEQVVAIASHDGGKQALETV
QRLLPVLCQAHGLTPEQVVAIASNGGGKQALETVQRLLPVLCQAHGLTPEQVVAIASHDGGKQALETVQRLLPVLCQA
HGLTPEQVVAIASNGGGKQALETVQRLLPVLCQAHGLTPEQVVAIASNIGGRPALESIVAQLSRPDPALAALTNDHLV
ALACLGGRPALDAVKKGLPHAPALIKRTNRRIPERTSHRVASGSGGGSGGDALDDFDLDMLGSDALDDFDLDMLGSDA
LDDFDLDMLGSDALDDFDLDMLSGGPKKKRKVGSQYLPDTDDRHRIEEKRKRTYETFKSIMKKSPFSGPTDPRPPPRR
IAVPSRSSASVPKPAPQPYPFTSSLSTINYDEFPTMVFPSGQISQASALAPAPPQVLPQAPAPAPAPAMVSALAQAPA
PVPVLAPGPPQAVAPPAPKPTQAGEGTLSEALLQLQFDDEDLGALLGNSTDPAVFTDLASVDNSEFQQLLNQGIPVAP
HTTEPMLMEYPEAITRLVTGAQRPPDPAPAPLGAPGLPNGLLSGDEDFSSIADMDFSALLGSGSGSRDSREGMFLPKP
EAGSAISDVFEGREVCQPKRLRPFHPPGSPWANRPLPASLAPTPTGPVHEPVGSLTPAPVPQPLDPAPAVTPEASHLL
EDPDEETSQAVKALREMADTVIPQKEEAAICGQMDLSHPPPRGHLDELTTTLESMTEDLNLDSPLTPELNEILDTFLN
DECLLHAMHISTGLSIFDTSLFSGGKRPAATKKAGQAKKKKGSYPYDVPDYA

Fusion Molecules

In some embodiments, a modulating agent may be or comprise a fusion molecule, such as a fusion molecule that comprises two or more moieties. In some embodiments, a fusion molecule comprises one or more moieties described herein, e.g., a targeting moiety and/or effector moiety. In some embodiments, a fusion molecule comprises one or more moieties covalently connected to one another. In some embodiments, the one or more moieties of a fusion molecule are situated on a single polypeptide chain, e.g., the polypeptide portions of the one or more moieties are situated on a single polypeptide chain.

In some embodiments, for example, a fusion molecule may comprise (e.g., as part of an effector and/or targeting moiety), dCas9-DNMT (e.g., comprises dCas9 and DNMT as part of the same polypeptide chain without regard to order), dCas9-p300, dCas9-VP64, dCas9-VPR, dCas9-DNMT-3a-3L, dCas9-DNMT-3a-3a, dCas9-DNMT-3a-3L-3a, dCas9-DNMT-3a-3L-KRAB, dCas9-KRAB, dCas9-APOBEC, APOBEC-dCas9, dCas9-APOBEC-UGI, dCas9-UGI, UGI-dCas9-APOBEC, UGI-APOBEC-dCas9, dCas9-VP64-RelA, dCas9-VPR-RelA, dCas9-VP64-p65, dCas9-VPR-p65, dCas9-VP64-RelA-p65, dCas9-VPR-RelA-p65, ZFM-VP64-RelA (where ZFM stands for Zn finger molecule), ZFM-VPR-RelA, ZFM-VP64-p65, ZFM-VPR-p65, ZFM-VP64-RelA-p65, ZFM-VPR-RelA-p65, TEM-VP64-RelA (where TEM stands for Tal effector molecule), TEM-VPR-RelA, TEM-VP64-p65, TEM-VPR-p65, TEM-VP64-RelA-p65, TEM-VPR-RelA-p65, or any variation of protein fusions as described herein, or other fusions of proteins or protein domains described herein.

Exemplary dCas9 fusion methods and compositions that are adaptable to methods and compositions provided by the present disclosure are known and are described, e.g., in Kearns et al., Functional annotation of native enhancers with a Cas9—histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067. Using methods known in the art, dCas9 can be fused to any of a variety of agents and/or molecules as described herein; such resulting fusion molecules can be useful in various disclosed methods.

In some embodiments, a fusion molecule may be or comprise a peptide oligonucleotide conjugate. Peptide oligonucleotide conjugates include chimeric molecules comprising a nucleic acid moiety covalently linked to a peptide moiety (such as a peptide/nucleic acid mixmer). In some embodiments, a peptide moiety may include any peptide or protein moiety described herein. In some embodiments, a nucleic acid moiety may include any nucleic acid or oligonucleotide, e.g., DNA or RNA or modified DNA or RNA, described herein.

In some embodiments, a peptide oligonucleotide conjugate comprises a peptide antisense oligonucleotide conjugate. In some embodiments, a peptide oligonucleotide conjugate is a synthetic oligonucleotide with a chemically modified backbone. A peptide oligonucleotide conjugate can bind to both DNA and RNA targets in a sequence-specific manner to form a duplex structure. When bound to double-stranded DNA (dsDNA) target, a peptide oligonucleotide conjugate replaces one DNA strand in a duplex by strand invasion to form a triplex structure and a displaced DNA strand may exist as a single-stranded D-loop.

In some embodiments, a peptide oligonucleotide conjugate may be cell- and/or tissue-specific. In some embodiments, such a conjugate may be conjugated directly to, e.g. oligos, peptides, and/or proteins, etc.

In some embodiments, a peptide oligonucleotide conjugate comprises a membrane translocating polypeptide, for example, membrane translocating polypeptides as described elsewhere herein.

Solid-phase synthesis of several peptide-oligonucleotide conjugates has been described in, for example, Williams, et al., 2010, Curr. Protoc. Nucleic Acid Chem., Chapter Unit 4.41, doi: 10.1002/0471142700.nc0441s42. Synthesis and characterization of very short peptide-oligonucleotide conjugates and stepwise solid-phase synthesis of peptide-oligonucleotide conjugates on new solid supports have been described in, for example, Bongardt, et al., Innovation Perspect. Solid Phase Synth. Comb. Libr., Collect. Pap., Int. Symp., 5th, 1999, 267-270; Antopolsky, et al., Helv. Chim. Acta, 1999, 82, 2130-2140.

In some embodiments, provided compositions are pharmaceutical compositions comprising fusion molecules as described herein.

In some aspects, the present disclosure provides cells or tissues comprising fusion molecules as described herein.

In some aspects, the present disclosure provides pharmaceutical compositions comprising fusion molecules as described herein.

Linkers

In some embodiments, modulating agents e.g., fusion molecules, may include one or more linkers. In some embodiments, a modulating agent, e.g., fusion molecule, comprising a first moiety and a second moiety has a linker between the first and second moieties, e.g., between a targeting moiety and an effector moiety. A linker may be a chemical bond, e.g., one or more covalent bonds or non-covalent bonds. In some embodiments linkers are covalent. In some embodiments, linkers are non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first moiety from a second, e.g., a targeting moiety from an effector moiety. In some embodiments, for example, a linker can be positioned between a targeting moiety and an effector moiety, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g. polypeptide) from a modulating agent, but sufficiently stable to prevent premature cleavage.

In some embodiments, one or more moieties of a modulating agent described herein are linked with one or more linkers.

As will be known by one of skill in the art, commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of a linker in aqueous solutions by forming hydrogen bonds with water molecules, and therefore reduce unfavorable interactions between a linker and protein moieties.

Rigid linkers are useful to keep a fixed distance between domains and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linking molecules include a hydrophobic linker, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a modulating agent (e.g. two polypeptides). Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of a modulating agent may be linked using charge-based chemistry, such that a positively charged component of a modulating agent is linked to a negative charge of another component or nucleic acid.

In some embodiments, a modulating agent e.g., fusion molecule, has the capacity to form linkages, e.g., after administration (e.g. to a subject), to other polypeptides, to another moiety as described herein, e.g., an effector molecule, e.g., a nucleic acid, protein, peptide or other molecule, or other agents, e.g., intracellular molecules, such as through covalent bonds or non-covalent bonds. In some embodiments, one or more amino acids on a polypeptide of a modulating agent are capable of linking with a nucleic acid, such as through arginine forming a pseudo-pairing with guanosine or an internucleotide phosphate linkage or an interpolymeric linkage. In some embodiments, a nucleic acid is a DNA such as genomic DNA, RNA such as tRNA or mRNA molecule. In some embodiments, one or more amino acids on a polypeptide are capable of linking with a protein or peptide.

In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Genomic Complex Modulation

In some embodiments, a modulating agent modulates (e.g., promotes or disrupts) one or more aspects of a genomic complex (e.g., ASMC) associated with a target gene (e.g., FXN). In some embodiments, modulation is or comprises modulation of a topological structure of a genomic complex (e.g., ASMC). In some embodiments, modulation of a topological structure of a genomic complex results in altered (e.g., increased) expression of a target gene (e.g., FXN). In some embodiments, no detectable modulation of a topological structure is observed, but altered expression of a target gene (e.g., FXN) is nonetheless observed. In some embodiments, modulation is or comprises binding to a component of the genomic complex (e.g., ASMC), e.g., a genomic sequence element. Binding may result in sequestering of the component and the level or occupancy of the genomic complex (e.g., ASMC), e.g., at a target gene (e.g., FXN), is thereby altered.

Those skilled in the art will appreciate that, in certain instances, two or more genomic complexes (e.g., ASMCs) may compete with each other with respect to a particular genomic region or particular genomic location (e.g., the FXN gene or an expression control sequence operably linked thereto). In some embodiments, disruption of one (a “first”) genomic complex (e.g., ASMC) may be achieved by stabilization of one or more other genomic complexes (e.g., ASMCs) that represent alternative (relative to the first genomic complex) structures available to the particular genomic region or location. In some embodiments, stabilization of one (a “first”) genomic complex (e.g., ASMC) may be achieved by disruption of one or more other genomic complexes (e.g., ASMCs) that represent alternative (relative to the first genomic complex) structures available to the particular genomic region or location. Thus, in some embodiments, disruption or stabilization of a genomic complex (e.g., ASMC) of interest may be achieved by targeting one or more competing genomic complexes for stabilization or disruption respectively (optionally without also providing a modulating agent that disrupts or stabilizes the genomic complex (e.g., ASMC) of interest).

A modulating agent may bind its target component of a genomic complex (e.g., ASMC) and alter formation of the genomic complex (e.g., by altering affinity of the targeted component to one or more other complex components, e.g., by at least 10%, 15%, 20%, 25%, 30%, 35%, 40%, 45%, 50%, 55%, 60%, 65%, 70%, 75%, 80%, 85%, 90%, 95%, or more). Alternatively or additionally, in some embodiments, binding by a modulating agent alters topology of genomic DNA impacted by a genomic complex, e.g., at least 10%, 15%, 20%, 25%, 30%, 35%, 40%, 45%, 50%, 55%, 60%, 65%, 70%, 75%, 80%, 85%, 90%, 95%, or more. In some embodiments, a modulating agent alters expression of a gene associated with a targeted genomic complex (e.g., ASMC) by at least 10%, 15%, 20%, 25%, 30%, 35%, 40%, 45%, 50%, 55%, 60%, 65%, 70%, 75%, 80%, 85%, 90%, 95%, or more. Changes in genomic complex formation, affinity of targeted components for other complex components, and/or changes in topology of genomic DNA impacted by a genomic complex may be evaluated, for example, using HiChIP, ChIAPET, 4C, or 3C, e.g., HiChIP.

A modulating agent as described herein comprises a targeting moiety. In some embodiments, a targeting moiety binds to a target genomic complex (e.g., ASMC) component (e.g., a genomic sequence element). In some embodiments, interaction between a targeting moiety and its targeted component interferes with one or more other interactions that the targeted component would otherwise make. In some embodiments, a modulating agent physically interferes with formation and/or maintenance of a genomic complex (e.g., ASMC), e.g., via the binding of the targeting moiety to its target genomic complex component. In some embodiments, the one or more other interactions that the targeted component would otherwise make are with polypeptide components of the genomic complex (e.g., ASMC) or with transcription machinery (e.g., transcription activating proteins or transcription repressing proteins).

In some embodiments, a modulating agent is complex-specific. That is, in some embodiments, a targeting moiety binds specifically to its target component, e.g., genomic sequence element, in one or more target genomic complexes (e.g., within a cell) and not to non-targeted genomic complexes (e.g., within the same cell). In some embodiments, a modulating agent specifically targets a genomic complex that is present in only certain cell types and/or only at certain developmental stages or times. In some embodiments, modulating agent binding to a target component of a genomic complex (e.g., ASMC) associated with or comprising a target gene (e.g., FXN) or a genomic sequence element operably linked to the target gene comprises changing (e.g., decreasing) the frequency and/or duration of association between a polypeptide component of the genomic complex and the operably linked genomic sequence element.

### Nucleating Polypeptides

In some embodiments, interaction between a targeting moiety and its targeted component or the function of an effector moiety interferes with one or more other interactions that the targeted component would otherwise make with a polypeptide component of a genomic complex (e.g., ASMC). In some embodiments, a polypeptide component is or comprises a nucleating polypeptide. A nucleating polypeptide may promote formation of an anchor sequence-mediated conjunction. Nucleating polypeptides that may be targeted by modulating agents as described herein may include, for example, proteins (e.g., CTCF, USF1, YY1, TAF3, ZNF143, etc) that bind specifically to anchor sequences, or other proteins (e.g., transcription factors) whose binding to a particular genomic sequence element may initiate formation of a genomic complex (e.g., ASMC) as described herein. In some embodiments, a modulating agent may target one or more anchor sequences or genomic sequence elements to which nucleating polypeptides may bind in a target genomic complex (e.g., ASMC). In some embodiments, a modulating agent may target (e.g., bind) to a nucleating polypeptide.

A nucleating polypeptide may be, e.g., CTCF, cohesin, USF1, YY1, TATA-box binding protein associated factor 3 (TAF3), ZNF143 binding motif, or another polypeptide that promotes formation of an anchor sequence-mediated conjunction. A nucleating polypeptide may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A nucleating polypeptide may modulate DNA interactions within or around the anchor sequence-mediated conjunction. For example, a nucleating polypeptide can recruit other factors to an anchor sequence, such that alteration (e.g. disruption) of an anchor sequence-mediated conjunction occurs.

A nucleating polypeptide may also have a dimerization domain for homo- or heterodimerization. One or more nucleating polypeptides, e.g., endogenous and engineered, may interact to form an anchor sequence-mediated conjunction. In some embodiments, a modulating agent disrupts a target genomic complex (e.g., ASMC) by interfering with (e.g. directly or indirectly) this interaction. In some embodiments, a nucleating polypeptide is engineered to further include a stabilization domain, e.g., cohesion interaction domain, to stabilize an anchor sequence-mediated conjunction. In some embodiments, a nucleating polypeptide is engineered to bind a target sequence, e.g., target sequence binding affinity is modulated. In some embodiments, a nucleating polypeptide is selected or engineered with a selected binding affinity for an anchor sequence within an anchor sequence-mediated conjunction.

Nucleating polypeptides and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, a nucleating polypeptide has a binding affinity for an anchor sequence greater than or less than a reference value, e.g., binding affinity for an anchor sequence in absence of an alteration. In some embodiments, a nucleating polypeptide is modulated to alter (e.g. disrupt) its interaction with an anchor sequence-mediated conjunction, e.g. its binding affinity for an anchor sequence within an anchor sequence-mediated conjunction.

### Transcription Machinery

In some embodiments, interaction between a targeting moiety and its targeted component or the function of an effector moiety interferes with one or more other interactions that the targeted component would otherwise make with components of the transcription machinery of the cell. Those skilled in the art are familiar with proteins that participate as part of the transcription machinery involved in transcribing a particular gene (e.g., a protein-coding gene). For example, RNA polymerase (e.g., RNA polymerase II), general transcription factors such as TFIIA, TFIIB, TFIID, TFIIE, TFIIF, and TFIIH, Mediator, certain elongation factors, etc.

In some embodiments, interaction between a targeting moiety and its targeted component or the function of an effector moiety promotes interactions of the targeted component (e.g., the genomic sequence element, e.g., an expression control sequence operably linked to a target gene) and/or the target gene (e.g., FXN) with components of the transcription machinery of the cell. Those skilled in the art are familiar with proteins that participate as part of the transcription machinery involved in transcribing a particular gene (e.g., a protein-coding gene). For example, RNA polymerase (e.g., RNA polymerase II), general transcription factors such as TFIIA, TFIIB, TFIID, TFIIE, TFIIF, and TFIIH, Mediator, certain elongation factors, etc.

### Transcription Regulators

In some embodiments, a modulating agent alters the interaction of a transcription regulatory protein with a target gene (e.g., FXN) and/or a genomic sequence element operably linked to the target gene (e.g., the target component of a targeting moiety). In some embodiments, a modulating agent promotes interaction of a transcription regulatory protein with a target gene (e.g., FXN) and/or a genomic sequence element operably linked to the target gene (e.g., the target component of a targeting moiety). In some embodiments, a modulating agent interferes with (e.g., inhibits) interaction of a transcription regulatory protein with a target gene (e.g., FXN) and/or a genomic sequence element operably linked to the target gene (e.g., the target component of a targeting moiety), e.g., by preventing the transcription regulatory protein from interacting with one or more other components of a genomic complex (e.g., ASMC) comprising or associated with the target gene (or a genomic sequence element operably linked thereto).

Those skilled in the art are aware of a large variety of transcriptional regulatory proteins, many of which are DNA binding proteins (e.g., containing a DNA binding domain such as a helix-loop-helix motif, ETS, a forkhead, a leucine zipper, a Pit-Oct-Unc domain, and/or a zinc finger), many of which interact with core transcriptional machinery by way of interaction with Mediator. In some embodiments, a transcriptional regulatory protein may be or comprise an activator (e.g., that may bind to an enhancer). In some embodiments, a transcriptional regulatory protein may be or comprise a repressor (e.g., that may bind to a silencer).

### Anchor Sequence-Mediated Conjunction (ASMC)

In some embodiments, a genomic complex modulated by a modulating agent of the present disclosure is or comprises an anchor sequence-mediated conjunction (ASMC). In some embodiments, an anchor sequence-mediated conjunction is formed when nucleating polypeptide(s) bind to anchor sequences in the genome and interactions between and among these proteins and, optionally, one or more other components (e.g., polypeptide components and/or non-genomic nucleic acid components), forms a conjunction in which the anchor sequences are physically co-localized. In some embodiments, one or more genes (e.g., the target gene, e.g., FXN) is associated with an anchor sequence-mediated conjunction. In some embodiments, the anchor sequence-mediated conjunction includes one or more anchor sequences, one or more genes, and one or more expression control sequences, such as an enhancing or silencing sequence. In some embodiments, a expression control sequence is within, partially within, or outside an anchor sequence-mediated conjunction.

In some embodiments, a genomic complex (e.g., an anchor sequence-mediated conjunction) comprises a first anchor sequence, a nucleic acid sequence (e.g., a gene), a expression control sequence, and a second anchor sequence. In some embodiments, a genomic complex (e.g., ASMC) comprises, in order: a first anchor sequence, a expression control sequence, and a second anchor sequence; or a first anchor sequence, a nucleic acid sequence (e.g., a gene), and a second anchor sequence. In some embodiments, either one or both of the nucleic acid sequence (e.g., gene) and the expression control sequence is located within or outside the genomic complex (e.g., ASMC). expression control sequence In some embodiments, a genomic complex (e.g., an anchor sequence-mediated conjunction) includes a TATA box, a CAAT box, a GC box, or a CAP site.

In some embodiments, a genomic complex (e.g., ASMC) colocalizes two genomic sequence elements (e.g., anchor sequences) that are outside of, not part of, not comprised within, or non-contiguous with (i) a gene whose expression is modulated (e.g., decreased or increased) by the formation or disruption of the genomic complex; and/or (ii) one or more expression control sequences operably linked to the gene.

In some embodiments, a genomic complex (e.g., ASMC) colocalizes two genomic sequence elements that are within, partially within, or contiguous with (i) a gene whose expression is modulated (e.g., decreased or increased) by the formation or disruption of the genomic complex; and/or (ii) one or more expression control sequences operably linked to the gene.

expression control sequence In some embodiments, a modulating agent may modulate transcription of a target gene associated with an ASMC. For example, in some embodiments, transcription of a target gene is activated by its inclusion in an activating ASMC or exclusion from a repressive ASMC; in some embodiments a modulating agent causes a target gene to be included in an activating ASMC or excluded from a repressive ASMC. In some embodiments, a modulating agent may cause an anchor sequence-mediated conjunction to comprise a expression control sequence that increases transcription of a nucleic acid sequence (e.g., gene), where the ASMC did not comprise the expression control sequence prior to modulation. In some embodiments, a modulating agent may cause an anchor sequence-mediated conjunction to exclude a expression control sequence that decreases transcription of a nucleic acid sequence (e.g., gene), where the ASMC comprised the expression control sequence prior to modulation.

In some embodiments, transcription of a target gene is repressed by its inclusion in a repressive ASMC or exclusion from an activating ASMC. In some such embodiments, a modulating agent causes a target gene to be excluded from an activating ASMC or included in a repressive ASMC. In some embodiments, an anchor sequence-mediated conjunction includes a expression control sequence that decreases transcription of a nucleic acid sequence (e.g., gene). In some embodiments, an anchor sequence-mediated conjunction excludes a expression control sequence that increases transcription of a nucleic acid sequence (e.g., gene).

An “activating ASMC” is an ASMC that is open to active gene transcription, for example, an ASMC comprising a expression control sequence (e.g., a promoter or enhancer) that enhances transcription of an operably linked nucleic acid sequence (e.g., gene). A “repressive ASMC”, is an ASMC that is closed off from active gene transcription, for example, an ASMC comprising a expression control sequence (e.g., a repressor sequence) that represses transcription of an operably linked nucleic acid sequence (e.g., gene). In some embodiments, an ASMC (e.g., an activating ASMC) comprises a gene and an operably linked enhancer and the gene is actively expressed. In some embodiments, an ASMC (e.g., an activating ASMC) comprises a gene and a repressor sequence is situated outside the ASMC, wherein the gene is actively expressed. In some embodiments, an ASMC (e.g., a repressive ASMC) comprises a gene and an operably linked repressor sequence situated within the ASMC and the gene is not actively expressed. In some embodiments, an ASMC (e.g., a repressive ASMC) comprises a gene and an enhancer is situated outside the ASMC, wherein the gene is not actively expressed. In some embodiments, an ASMC (e.g., an activating ASMC) comprises a gene and an operably linked enhancer, wherein a repressor is situated outside the ASMC and the gene is actively expressed. In some embodiments, an ASMC (e.g., a repressive ASMC) comprises a gene and an operably linked repressor sequence, wherein an enhancer situated outside the ASMC and the gene is not actively expressed.

In some embodiments, a target gene is non-contiguous with one or more expression control sequences. In some embodiments where a gene is non-contiguous with its expression control sequence(s), a gene may be separated from one or more expression control sequences by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a gene is separated from a expression control sequence by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

Without wishing to be bound by theory, it is contemplated that in some embodiments, understanding (e.g., identifying or classifying) whether an ASMC is or corresponds to a particular type of anchor sequence-mediated conjunction may help to determine how to modulate gene expression by altering the ASMC, e.g., influencing the choice of DNA-binding moiety or effector moiety. For example, in some embodiments, some types of anchor sequence-mediated conjunctions comprise one or more expression control sequences (e.g., an enhancer) within an anchor sequence-mediated conjunction. Modulation (e.g., disruption) of such an ASMC by modulating the genomic complex comprising the ASMC and/or modulating presence of the ASMC within a genomic complex, e.g., altering one or more anchor sequences wherein such an alteration results in a disrupted ASMC, is likely to decrease transcription of a target gene within the genomic complex and/or ASMC. In some embodiments, modulation (e.g., disruption) of a repressive ASMC, or a genomic complex comprising the ASMC, results in increased gene expression. In some embodiments, modulation (e.g., disruption) of an activating ASMC, or a genomic complex comprising the ASMC, results in decreased gene expression.

### Compositions: Methods of Making, Formulation, Delivery, and Administration

The present disclosure, among other things, provides compositions that comprise a modulating agent described herein, and/or compositions that deliver a modulating agent to a cell, tissue, organ, and/or subject. In some embodiments, a modulating agent that comprises a polypeptide (e.g., a moiety that is or comprises a polypeptide) may be provided via a composition that includes the polypeptide or polypeptide portion of the modulating agent as a polypeptide, or alternatively via a composition that includes a nucleic acid encoding the modulating agent or polypeptide portion(s) thereof, and associated with sufficient other sequences to achieve expression of the modulating agent or polypeptide portion(s) thereof in a system of interest (e.g., in a particular cell, tissue, organism, etc).

In some embodiments, a provided composition may be a pharmaceutical composition whose active ingredient comprises or delivers a modulating agent as described herein and is provided in combination with one or more pharmaceutically acceptable excipients, optionally formulated for administration to a subject (e.g., to a cell, tissue, or other site thereof).

In some aspects, the present disclosure provides methods of delivering a therapeutic comprising administering a composition as described herein to a subject, wherein a genomic complex modulating agent is a therapeutic and/or wherein delivery of a therapeutic targets genomic complexes (e.g., ASMCs) characterized by an integrity index to change gene expression relative to gene expression in absence of a therapeutic.

In some aspects, a system for pharmaceutical use comprises a composition that targets a genomic complex characterized by an integrity index by disrupting a genomic complex. In some embodiments, the composition targets the genomic complex by binding an anchor sequence in the genomic complex to alter formation of an anchor sequence-mediated conjunction, wherein such a composition modulates transcription, in a human cell, of a target gene associated with the anchor sequence-mediated conjunction.

Thus, in some embodiments, the present disclosure provides compositions comprising a modulating agent (e.g., disrupting agent), or a production intermediate thereof. In some particular embodiments, the present disclosure provides compositions of nucleic acids that encode a modulating agent (e.g., disrupting agent) or polypeptide portion thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc). In some embodiments, provided nucleic acids that encode a modulating agent (e.g., disrupting agent) or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc).

In some embodiments, a modulating agent (e.g., disrupting agent) is or comprises a vector, e.g., a viral vector, comprising one or more nucleic acids encoding one or more components of a modulating agent (e.g., disrupting agent) as described herein.

Production

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter, and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not interpreted to be limited to use of any particular promoter or category of promoters (e.g. constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate expression control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of expression control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

In some embodiments, a modulating agent comprises or is a protein and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise nontranscribed elements such as an origin of replication, a suitable promoter, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer 2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Proteins comprise one or more amino acids. Amino acids include any compound and/or substance that can be incorporated into a polypeptide chain, e.g., through formation of one or more peptide bonds. In some embodiments, an amino acid has the general structure H₂N— C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Delivery

In various embodiments compositions described herein (e.g., modulating agents) are pharmaceutical compositions. In some embodiments, compositions (e.g. pharmaceutical compositions) described herein may be formulated for delivery to a cell and/or to a subject via any route of administration. Modes of administration to a subject may include injection, infusion, inhalation, intranasal, intraocular, topical delivery, intercannular delivery, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a genomic complex (e.g., ASMC) modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof.

In some embodiments, a composition of the present disclosure crosses a blood-brain-barrier, a placental membrane, or a blood-testis barrier.

In some embodiments, a composition as provided herein is administered systemically.

In some embodiments, administration is non-parenteral and a therapeutic is a parenteral therapeutic.

Pharmaceutical Compositions

As used herein, the term “pharmaceutical composition” refers to an active agent (e.g., disrupting agent), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, a composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to a therapeutic alone. In some embodiments, a composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to a therapeutic alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to a therapeutic alone.

Pharmaceutical compositions described herein may be formulated for example including a carrier, such as a pharmaceutical carrier and/or a polymeric carrier, e.g., a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. Jul. 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

Pharmaceutical uses of the present disclosure may include compositions (e.g. modulating agents, e.g., disrupting agents) as described herein. In some aspects, a system for pharmaceutical use comprises: a protein comprising a first polypeptide domain, e.g., a Cas or modified Cas protein, and a second polypeptide domain, e.g., a polypeptide having DNA methyltransferase activity or associated with demethylation or deaminase activity, in combination with at least one guide RNA (gRNA) or antisense DNA oligonucleotide that targets an ncRNA, such as an eRNA. A system is effective to alter, in at least a human cell, a genomic complex, e.g., a target anchor sequence-mediated conjunction, characterized by an integrity index.

In some embodiments, pharmaceutical compositions of the present disclosure comprise a zinc finger nuclease (ZFN), or a mRNA encoding a ZFN, that targets (e.g., cleaves) an ncRNA, such as an eRNA.

In some aspects, a system for pharmaceutical use comprises a composition that binds an ncRNA, such as an eRNA, and alters formation of a genomic complex comprising the ncRNA (e.g., eRNA), e.g., an anchor sequence-mediated conjunction, (e.g., a genomic complex characterized by an integrity index) wherein such a composition modulates transcription, in a human cell, of a target gene associated with the genomic complex, e.g., anchor sequence-mediated conjunction.

In some aspects, a system for altering, in a human cell, expression of a target gene, comprises a targeting moiety (e.g., a gRNA, a membrane translocating polypeptide) that associates with an ncRNA, such as an eRNA, associated with a target gene, and an effector moiety (e.g. an enzyme, e.g., a nuclease or deactivated nuclease (e.g., a Cas9, dCas9), a methylase, a de-methylase, a deaminase) operably linked to the targeting moiety, wherein the system is effective to alter (e.g., decrease) expression of the target gene. The targeting moiety and effector moiety may be different and separate (e.g., comprised in different physical portions of a disrupting agent) moieties. A targeting moiety and an effector moiety may be linked, e.g., covalently, e.g., by a linker. In some embodiments, a system comprises a synthetic polypeptide comprising a targeting moiety and an effector moiety. In some embodiments, a system comprises a nucleic acid vector or vectors encoding at least one of a targeting moiety and an effector moiety.

In some aspects, pharmaceutical compositions may comprise a composition that targets a genomic complex (e.g., ASMC) characterized by an integrity index by binding an anchor sequence of an anchor sequence-mediated conjunction and altering formation of an anchor sequence-mediated conjunction, wherein the composition modulates transcription, in a human cell, of a target gene associated with the genomic complex (e.g., ASMC). In some embodiments, a composition targets a genomic complex characterized by an integrity index by disrupting formation of an anchor sequence-mediated conjunction (e.g., decreases affinity of an anchor sequence to a conjunction nucleating molecule, e.g., at least 10%, 15%, 20%, 25%, 30%, 35%, 40%, 45%, 50%, 55%, 60%, 65%, 70%, 75%, 80%, 85%, 90%, 95%, or more). In some embodiments, disrupting formation comprises an alteration of integrity index by modulating affinity of an anchor sequence to a conjunction nucleating molecule, e.g., by at least 10%, 15%, 20%, 25%, 30%, 35%, 40%, 45%, 50%, 55%, 60%, 65%, 70%, 75%, 80%, 85%, 90%, 95%, or more.

In some embodiments, administration of compositions described herein improves at least one pharmacokinetic or pharmacodynamic parameter of at least one component of the composition (e.g. a pharmacoagent), such as targeting, absorption, and transport, as compared to another moiety alone, or reduces at least one toxicokinetic parameter, such as diffusion to non-target location, off-target activity, and toxic metabolism, as compared to another moiety alone (e.g., by at least 5%, 10%, 20%, 25%, 30%, 40%, 50%, 60%, 70%, 80% or more). In some embodiments, administration of compositions of the present disclosure increases a therapeutic range of at least one component of a modulating agent (e.g., by at least 5%, 10%, 20%, 25%, 30%, 40%, 50%, 60%, 70%, 80% or more). In some embodiments, administration of compositions provided herein reduces a minimum effective dose, as compared to another moiety alone (e.g., by at least 5%, 10%, 20%, 25%, 30%, 40%, 50%, 60%, 70%, 80% or more). In some embodiments, administration of compositions provided increases a maximum tolerated dose, as compared to a modulating agent alone (e.g., by at least 5%, 10%, 20%, 25%, 30%, 40%, 50%, 60%, 70%, 80% or more). In some embodiments, administration of compositions provided herein increases efficacy or decreases toxicity of a therapeutic, such as non-parenteral administration of a parenteral therapeutic. In some embodiments, administration of compositions provided herein increases a therapeutic range of a modulating agent while decreasing toxicity, as compared to a modulating agent alone (e.g., by at least 5%, 10%, 20%, 25%, 30%, 40%, 50%, 60%, 70%, 80% or more).

In some aspects, the present disclosure provides a modulating agent, e.g., a disrupting agent, comprising a targeting moiety that binds an ncRNA, such as an eRNA, and alters, e.g., decreases, formation of a genomic or transcription complex, e.g., an anchor sequence-mediated conjunction (e.g., decreases the level of the complex by at least 10%, 15%, 20%, 25%, 30%, 35%, 40%, 45%, 50%, 55%, 60%, 65%, 70%, 75%, 80%, 85%, 90%, 95%, or more).

In some embodiments, a gRNA is administered in combination with a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to an ncRNA, such as an eRNA. For example, in some embodiments, one gRNA is administered, e.g., to produce an inactivating indel mutation in an ncRNA, such as an eRNA, e.g., one gRNA is administered in combination with a nuclease, e.g., wtCas9.

In some aspects, the present disclosure provides a composition comprising a nucleic acid or combination of nucleic acids that when administered to a subject in need thereof introduce a site specific alteration (e.g., insertion, deletion (e.g., knockout), translocation, inversion, single point mutation) in a target sequence of a target genomic complex (e.g., ASMC) characterized by an integrity index or of a component of a target genomic complex, e.g., an ncRNA, eRNA, thereby modulating gene expression in a subject.

### Uses

The present disclosure is further directed to uses of the modulating agents disclosed herein. Among other things, in some embodiments such provided technologies may be used to achieve modulation, e.g., an increase, of target gene (e.g., FXN) expression and, for example, enable control of target gene activity, delivery, and penetrance, e.g., in a cell. In some embodiments, a cell is a mammalian, e.g., human, cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell. In some embodiments, a cell is a muscle cell (e.g., a muscle cell in the heart, e.g., a cardiomyocyte) or a neuronal cell (e.g., a cell of the central nervous system or a cell of the spine, e.g., a cell (e.g., neuron) of the dorsal root ganglia (DRG)).

In some embodiments, such provided technologies may be used to treat FRDA or a symptom associated with FRDA in a subject, e.g., a patient, in need thereof.

Modulating Gene Expression

The present disclosure is further directed, in part, to a method of modulating, e.g., increasing, expression of a target gene (e.g., FXN), comprising providing a modulating agent described herein (or a nucleic acid encoding the same, or pharmaceutical composition comprising said modulating agent or nucleic acid), and contacting a cell, the target gene, and/or an operably linked expression control element(s) with the modulating agent. In some embodiments, modulating, e.g., increasing, expression of a target gene comprises modulation of transcription of a target gene as compared with a reference value, e.g., transcription of a target gene the in absence of the modulating agent. In some embodiments, the method of modulating, e.g., increasing, expression of a target gene is used ex vivo, e.g., on a cell from a subject, e.g., a mammalian subject, e.g., a human subject. In some embodiments, the method of modulating, e.g., increasing, expression of a target gene are used in vivo, e.g., on a mammalian subject, e.g., a human subject. In some embodiments, the method of modulating, e.g., increasing, expression of a target gene are used in vitro, e.g., on a cell or cell line described herein.

The present disclosure is further directed, in part to a method of treating a condition associated with under-expression of a target gene (e.g., FXN) and/or pathologically low levels of a target gene (e.g., FXN) product (e.g., mRNA or protein) in a subject, comprising administering to the subject a modulating agent described herein (or a nucleic acid encoding the same, or pharmaceutical composition comprising said modulating agent or nucleic acid). Conditions associated with under-expression of particular genes and/or pathologically low levels of a gene product are known to those of skill in the art. Such conditions include, but are not limited to, FRDA (associated with under expression of FXN and/or pathologically low levels of FXN gene product), metabolic disorders, neuromuscular disorders, cancer (e.g., solid tumors), fibrosis, diabetes, urea disorders, immune disorders, inflammation, and arthritis.

The present disclosure is further directed, in part to a method of treating a condition associated with mis-regulation of the expression of a target gene in a subject, comprising administering to the subject an modulating agent described herein (or a nucleic acid encoding the same, or pharmaceutical composition comprising said modulating agent or nucleic acid). Without wishing to be bound by theory, it is thought that a modulating agent may be used to target (e.g., increase expression of) a gene which modulates the expression of a target gene, thus altering expression of the target gene by altering expression of the modulating gene. Conditions associated with mis-regulation of the expression of particular genes are known to those of skill in the art. Such conditions include, but are not limited to metabolic disorders, neuromuscular disorders, cancer (e.g., solid tumors), fibrosis, diabetes, urea disorders, immune disorders, inflammation, and arthritis.

Methods and compositions as provided herein may treat a condition by stably or transiently altering (e.g., increasing) transcription of a target gene (e.g., FXN). In some embodiments, such a modulation persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, the condition treated is FRDA. FRDA is a genetic, progressive, neurodegenerative movement disorder. In some embodiments, a subject, e.g., patient, treated with a method described herein, is a subject, e.g., patient, with FRDA. In some embodiments, FRDA has a typical age of onset between 10 and 15 years. In some embodiments, a subject, e.g., patient, is diagnosed with FRDA at an age under 25 years, e.g., between 10-15 years. In some embodiments, a subject, e.g., patient, is diagnosed with FRDA at an age of 26 to 39 years, e.g., a subject, e.g., a patient, is diagnosed with Late-onset FRDA (LOFA). In some embodiments, a subject, e.g., patient, is diagnosed with FRDA at an age of 40 years or greater, e.g., a subject, e.g., a patient, is diagnosed with Very late-onset FRDA (VLOFA). Initial symptoms may include unsteady posture, frequent falling, and progressive difficulty in walking due to impaired ability to coordinate voluntary movements (ataxia). Affected individuals may develop slurred speech (dysarthria), characteristic foot deformities, and/or an irregular curvature of the spine (scoliosis). FRDA may be associated with cardiomyopathy, a disease of cardiac muscle that may lead to heart failure or irregularities in heart rhythm (cardiac arrhythmias). About a third of patients with FRDA develop diabetes mellitus. In some embodiments, a method or composition provided herein ameliorates (e.g., makes less severe) or eliminates one or more symptom associated with FRDA chosen from, but not limited to, unsteady posture, frequent falling, difficulty walking, ataxia, dysarthria, foot deformity, scoliosis, cardiomyopathy, cardiac arrhythmia, or diabetes mellitus.

In some embodiments, a method provided herein may modulate, e.g., increase, expression of a target gene (e.g., FXN) by disrupting a genomic complex, e.g., an anchor sequence-mediated conjunction, associated with said target gene. A gene that is associated with an anchor sequence-mediated conjunction may be at least partially within a conjunction (that is, situated sequence-wise between a first and second anchor sequences), or it may be external to a conjunction in that it is not situated sequence-wise between a first and second anchor sequences, but is located on the same chromosome and in sufficient proximity to at least a first or a second anchor sequence such that its expression can be modulated by controlling the topology of the anchor sequence-mediated conjunction. Those of ordinary skill in the art will understand that distance in three-dimensional space between two elements (e.g., between the gene and the anchor sequence-mediated conjunction) may, in some embodiments, be more relevant than distance in terms of basepairs. In some embodiments, an external but associated gene is located within 2 Mb, within 1.9 Mb, within 1.8 Mb, within 1.7 Mb, within 1.6 Mb, within 1.5 Mb, within 1.4 Mb, with 1.3 Mb, within 1.3 Mb, within 1.2 Mb, within 1.1 Mb, within 1 Mb, within 900 kb, within 800 kb, within 700 kb, within 500 kb, within 400 kb, within 300 kb, within 200 kb, within 100 kb, within 50 kb, within 20 kb, within 10 kb, or within 5 kb of the first or second anchor sequence.

In some embodiments, modulating expression of a gene comprises altering accessibility of an expression control sequence to a gene. An expression control sequence, whether internal or external to an anchor sequence-mediated conjunction, can be an enhancing sequence or a silencing (or repressive) sequence.

Epigenetic Modification

The present disclosure is further directed, in part, to a method of epigenetically modifying a target gene (e.g., FXN), an expression control element operably linked to a target gene, or an anchor sequence (e.g., an anchor sequence proximal to a target gene or associated with an anchor sequence-mediated conjunction comprising or associated with a target gene or expression control sequence operably linked to said target gene), the method comprising providing a modulating agent or nucleic acid encoding the same or pharmaceutical composition comprising said modulating agent or nucleic acid; and contacting the target gene (e.g., FXN), an expression control sequence operably linked to the target gene, or a cell with the modulating agent, nucleic acid, or pharmaceutical composition, thereby epigenetically modifying the target gene or an expression control sequence operably linked to the target gene.

In some embodiments, a method of epigenetically modifying a target gene (e.g., FXN) or an expression control sequence operably linked to a target gene comprises increasing or decreasing DNA methylation of the target gene or an expression control sequence operably linked to a target gene. In some embodiments, a method of epigenetically modifying a target gene or a transcription control element operably linked to a target gene comprises increasing or decreasing histone methylation of a histone associated with the target gene or an expression control sequence operably linked to a target gene. In some embodiments, a method of epigenetically modifying a target gene or an expression control sequence operably linked to a target gene comprises increasing or decreasing histone acetylation of a histone associated with the target gene or an expression control sequence operably linked to a target gene. In some embodiments, a method of epigenetically modifying a target gene or an expression control sequence operably linked to a target gene comprises increasing or decreasing histone sumoylation of a histone associated with the target gene or an expression control sequence operably linked to a target gene. In some embodiments, a method of epigenetically modifying a target gene or an expression control sequence operably linked to a target gene comprises increasing or decreasing histone phosphorylation of a histone associated with the target gene or an expression control sequence operably linked to a target gene.

In some embodiments, a method of epigenetically modifying a target gene (e.g., FXN) or an expression control sequence operably linked to a target gene may decrease the level of the epigenetic modification by at least 10, 20, 30, 40, 50, 60, 70, 80, 90, or 100% (and optionally up to 100%) relative to the level of the epigenetic modification at that site in a cell not contacted by the composition or treated with the method. In some embodiments, a method of epigenetically modifying a target gene or an expression control sequence operably linked to a target gene may increase the level of the epigenetic modification by at least 10, 20, 30, 40, 50, 60, 70, 80, 90, 100, 120, 140, 160, 180, 200, 300, 400, 500, 600, 700, 800, 900, or 1000% (and optionally up to 200, 300, 400, 500, 600, 700, 800, 900, 1000, or 2000%) relative to the level of the epigenetic modification at that site in a cell not contacted by the composition or treated with the method. In some embodiments epigenetic modification of a target gene (e.g., FXN) or an expression control sequence operably linked to a target gene may modify the level of expression of the target gene, e.g., as described herein.

In some embodiments, an epigenetic modification produced by a method described herein persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, a modulating agent for use in a method of epigenetically modifying a target gene (e.g., FXN) or an expression control sequence operably linked to a target gene comprises an effector moiety that is or comprises an epigenetic modifying moiety.

For example, an effector moiety may be or comprise an epigenetic modifying moiety with histone acetyltransferase activity, and histone(s) associated with a target gene (e.g., FXN) or an expression control sequence operably linked to the target gene may be altered to increase their acetylation (e.g., increasing interaction of a transcription factor with a portion of target gene or expression control sequence, e.g., and thereby increasing transcription of the target gene).

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### EXAMPLES

### Example 1. Increasing FXN Expression Using a dCas9-p300 Modulating Agent

This example demonstrates the use of a modulating agent, e.g., fusion molecule, comprising a targeting moiety comprising a dCas9 molecule and an effector moiety comprising p300 to increase FXN expression and aconitase activity in FRDA patient-derived fibroblasts.

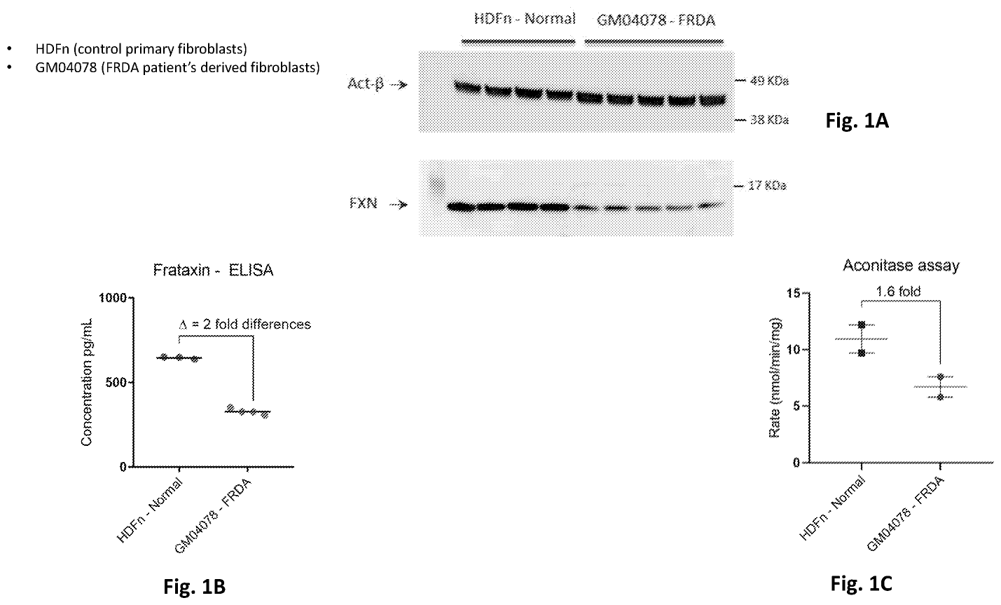

FXN protein levels are decreased in FRDA patient-derived fibroblasts (GM04078 cells, Coriell Institute) relative to control primary fibroblasts (HDFn) as seen by Western blot (FIG. 1A) and ELISA (FIG. 1B). Aconitase activity, associated with mitochondrial health, is decreased in cells derived from FRDA patients due to lower FXN levels, and this was confirmed by performing an aconitase activity assay (Abcam Kit ab109712) (FIG. 1C).

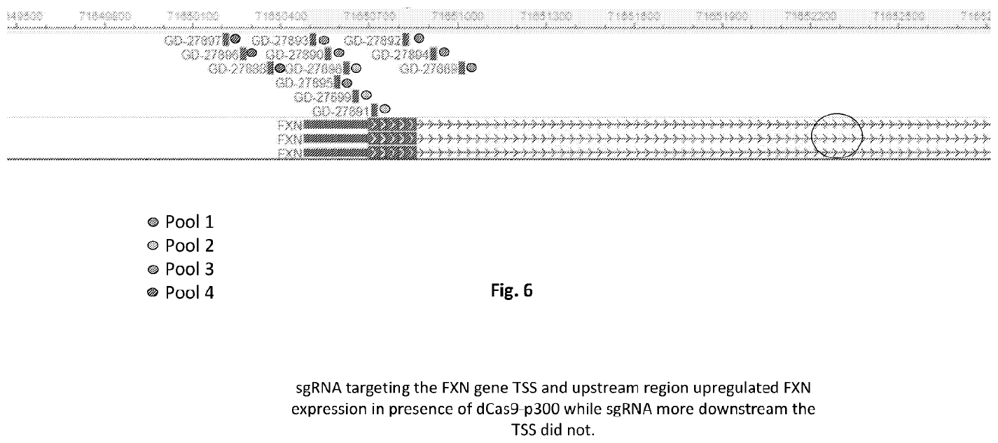

A modulating agent comprising a fusion molecule of a targeting moiety comprising a dCas9 molecule and an effector moiety comprising p300 was created, along with sgRNA(s), and FRDA fibroblasts were tested to see if the modulating agent could increase the FXN expression or aconitase activity of the cells. dCas9-p300 modulating agent was delivered to FRDA patient-derived fibroblasts in the form of mRNA encoding the modulating agent together with pools of sgRNA guides targeting a sequence starting around the FXN TSS (FIG. 6). The region targeted by the modulating agents included approximately 300 bp sequence upstream and 600 bp downstream of the TSS for FXN gene. sgRNA were used in pools of three different sgRNAs and co-delivered together with dCas9-p300 modulating agent encoding mRNA using a lipid nanoparticle (LNP) formulation (see Table 4). In addition, a non-targeting sgRNA and untreated cells were used as control in these experiments.

TABLE 4

| SEQ ID | | Upstream | Downstream | |
|---|---|---|---|---|
| NO: | sgRNA ID | End | End | Pool |
| 21 | GD-27899 | 71650645 | 71650667 | 2 |
| 22 | GD-27898 | 71650613 | 71650635 | 2 |
| 7 | GD-27897 | 71650204 | 71650226 | 4 |
| 8 | GD-27896 | 71650262 | 71650284 | 4 |
| 6 | GD-27895 | 71650582 | 71650604 | 1 |
| 23 | GD-27894 | 71650909 | 71650931 | 3 |
| 4 | GD-27893 | 71650500 | 71650522 | 1 |
| 24 | GD-27892 | 71650815 | 71650837 | 3 |
| 25 | GD-27891 | 71650708 | 71650730 | 2 |
| 6 | GD-27890 | 71650550 | 71650572 | 1 |
| 26 | GD-27889 | 71651006 | 71651028 | 3 |
| 9 | GD-27888 | 71650355 | 71650377 | 4 |

FRDA patient-derived fibroblasts (GM04078 cells) were seeded either in 12- or 6-well plates in alpha-MEM 15% FBS medium. The next day, 2-6 μg of LNPs in a 20-60 μl formulation were added to the cells (either mixed on the plate or in a tube prior to addition to addition to the cells). After 24 hours the medium containing LNPs was replaced with fresh medium, and samples collected at 48 and 72 hours after the initial treatment with LNPs.

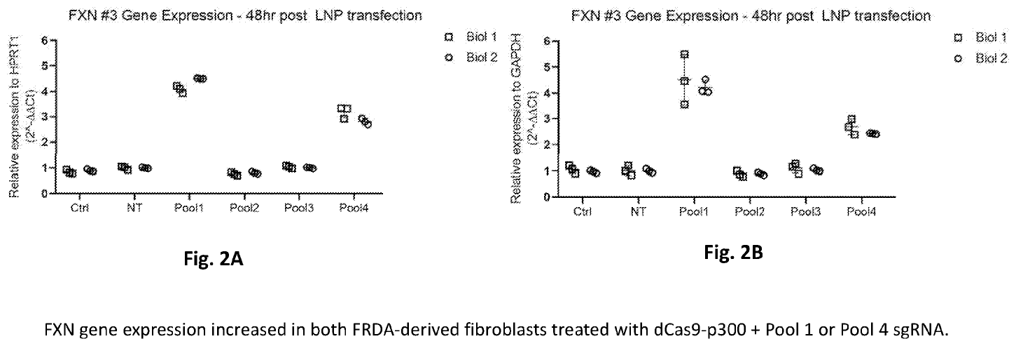

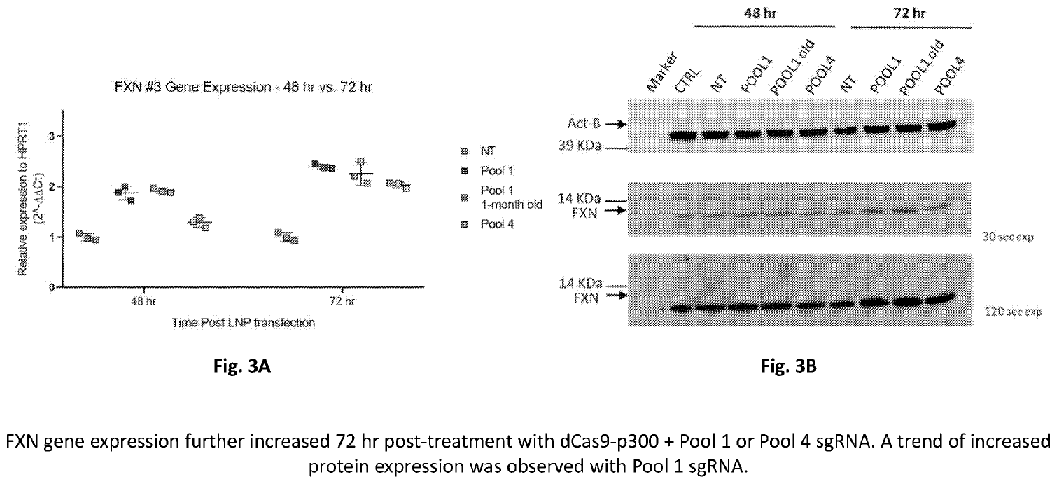

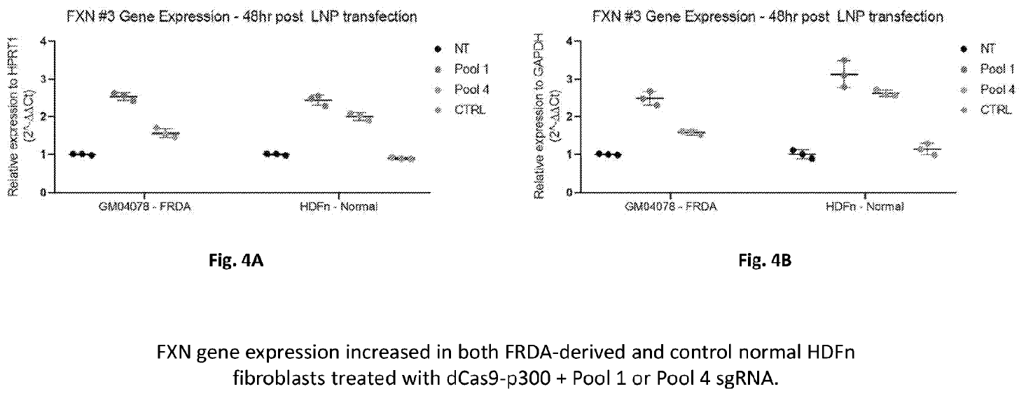

RNA was isolated from four independent experiments using the RNAeasy MiniKit (Qiagen) following the Manufacturer's protocol. RNA samples were retrotranscribed to cDNA using LunaScript RT SuperMix Kit (NEB) and analyzed by quantitative PCR (qPCR) using an FXN specific Taqman primer/probe set assay with the Taqman Fast Advanced Master Mix (Thermo Scientific). FXN expression was quantified relative to the expression of either HPRT1 or GAPDH reference genes using the AACt method, and the non-targeting sgRNA sample was used as calibrator. Data showed that delivery of two different sgRNA pools together with dCas9-p300 modulating agent increase FXN gene expression up to approximately 4.5-fold relative to the non-targeting sgRNA control (FIGS. 2A and 2B). The fold increase depended on the sgRNA and the concentration of LNPs used. The increase of FXN expression was observed at 48 hours as well as 72 hours (FIG. 3A) after initial LNP treatment with one sgRNA pool. The sgRNA pools that lead to the induction of FXN gene expression using dCas9-p300 modulating agent were located up to 300 bp upstream and 150 bp downstream of the TSS of FXN gene. In addition, these modulating agents also increase the expression of FXN in normal unaffected fibroblasts (HDFn, ATCC) (FIGS. 4A and 4B).

Protein lysates were obtained using RIPA buffer supplemented with a protease inhibitor Protein was quantified via BCA assay (BioVision). Samples were run on a Western Blot 4-12% Bis-Tris Gel (Thermo Fisher) and blotted using anti-Frataxin and anti-beta-Actin antibodies. Results showed an increased FXN protein expression for GM04078 fibroblasts treated with dCas9-p300 modulating agent plus sgRNA against the region 150 bp downstream the TSS of FXN gene, 72 hours post LNPs delivery (FIG. 3B).

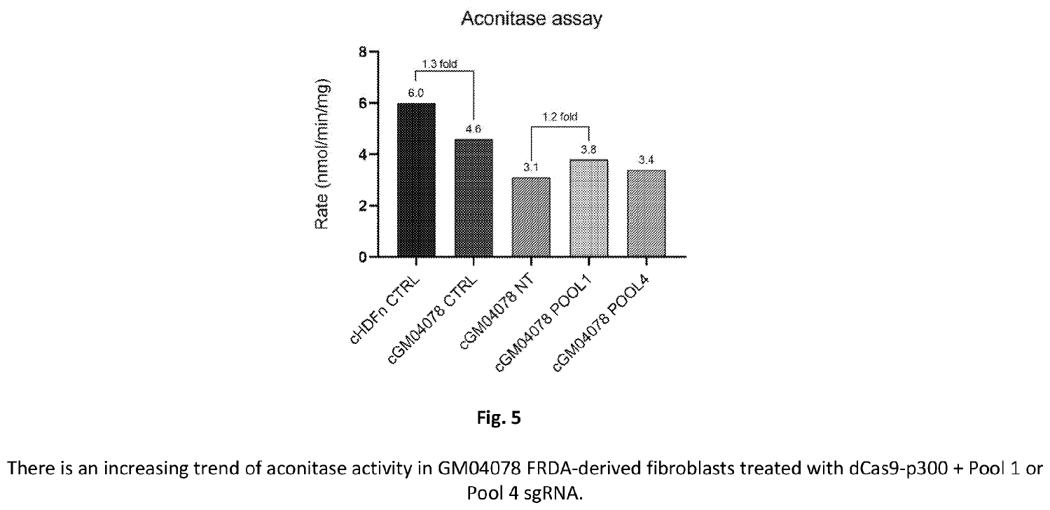

Aconitase activity, associated with mitochondrial health, is decreased in cells derived from FRDA patients due to lower FXN levels. It was hypothesized that increased FXN expression should increase aconitase enzyme activity. Aconitase enzyme activity was measured using Abcam Kit ab109712 with samples collected 72 hr post LNPs delivery of dCas9-p300 modulating agent targeted around the TSS of FXN gene. An increase in aconitase activity was observed in cells treated with this modulating agent targeted in regions around the TSS of FXN gene (FIG. 5).

TABLE 5
Sequences For Use with
dCas9-p300 Modulating Agent

| Guide Sequence | Genomic Coordinates |
|---|---|
| GCAAAGCACGGAGTGCAACC | chr9: |
| (SEQ ID NO: 4) | 71650500-71650522 |
| ATGCACGAATAGTGCTAAGC | chr9: |
| (SEQ ID NO: 5) | 71650550-71650572 |
| GCAGCTAGAGGTTAGACCTC | chr9: |
| (SEQ ID NO: 6) | 71650582-71650604 |
| GTTCCTACTTCATAGGATTG | chr9: |
| (SEQ ID NO: 7) | 71650204-71650226 |
| AGGTTAATTAACTTGCCCTC | chr9: |
| (SEQ ID NO: 8) | 71650262-71650284 |
| CTGCTGTAAACCCATACCGG | chr9: |
| (SEQ ID NO: 9) | 71650355-71650377 |

### Example 2. FXN Expression Changes in WT iPSCs

This example demonstrates the use of a modulating agent, e.g., fusion molecule, comprising a targeting moiety comprising a dCas9 molecule and an effector moiety comprising VPR to increase FXN expression in induced pluripotent stem cell derived cardiomyocytes.

WT iPSC-derived Cardiomyocytes (iCardiomyocytes) and WT iPSC-derived Glutamatergic Cortical Neurons (iNeuron) were treated with mRNA encoding a modulating agent comprising a fusion molecule comprising dCas9-p300 or dCas9-VPR co-delivered with either a single sgRNA or a pool of 3 sgRNAs (pool 1 from Example 1). The region targeted by the sgRNAs is about 100 bp upstream of the FXN gene TSS. RNAs were delivered using Lipofectamine MessengerMAX (ThermoFisher) following the manufacturer's protocol. In addition, a dCas9 without effector, a safe harbor (SH) sgRNA, and untreated cells were used as controls in these experiments.

iCardiomyocytes (Fujifilm Cellular Dynamics International [FCDI] cat #R1017) were seeded in 0.1% gelatin (Stemcell Technologies)-coated 24-well plates following the manufacturer's protocol and maintained in the recommended medium. 7 days post-plating, 0.5 ug of mRNA in LNPs formulation were added to the cells. After 24 hours, the medium was changed and samples collected at different timepoints (24-120 hours post-treatment with LNPs).

iNeurons (FCDI cat #R1034) were seeded in 24-well PDL Biocoat plates (Corning) coated with natural mouse laminin (ThermoFisher). Cells were maintained in BrainPhys Medium (Stemcell Technologies) supplemented with N2-A (Stemcell Technologies), iCell Neural Supplement B, and iCell Nervous System Supplement (FCDI). 7 days post-plating, 0.5 ug of mRNA in LNPs formulation were added to the cells. After 24 hours, the medium was changed and samples collected at different timepoints (24-96 hours post-treatment with LNPs).

RNA was purified from samples using the RNEasy MiniKit (Qiagen) following the manufacturer's protocol. RNA samples were retrotranscribed to cDNA using LunaScript RT SuperMix Kit (NEB) and analyzed by quantitative PCR (qPCR) using a FXN-specific Taqman assay with the Taqman Fast Advanced Master Mix (Thermo Scientific). Expression data was analyzed using the standard curve method. FXN expression was normalized to the expression of HPRT1, and fold-change calculated relative to the SH sgRNA-treated control for each timepoint. Fold-change was calculated by dividing normalized FXN mRNA quantity of each sample by the mean of the SH control group.

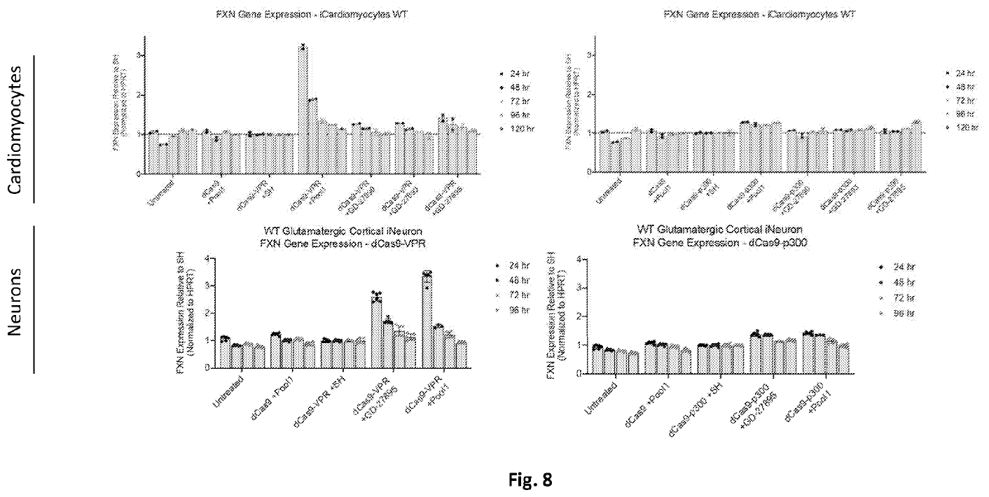

Results in FIG. 8 show that delivery of sgRNA pool1 together with dCas9-VPR modulating agent-encoding RNA in iCardiomyocytes upregulates FXN gene expression approximately 3-fold compared to dCas9-VPR plus SH control at 24 hr. The increase of FXN was observed at 48 hr post-LNP delivery as well. The single sgRNA GD-27895 together with dCas9-VPR modulating agent-encoding mRNA increased FXN expression only marginally in iCardiomyocytes. Modulating agent comprising a fusion molecule comprising dCas9-p300 also slightly upregulated FXN in WT iCardiomyocytes about 1.3-fold using pool1.

In Glutamatergic iNeurons, sgRNA pool1 with dCas9-VPR modulating agent-encoding mRNA upregulated FXN gene expression approximately 3-fold compared to the SH control at 24 hr. The increase of FXN was observed at 48 hr post-LNP delivery, as well. The single sgRNA GD-27895 together with dCas9-VPR modulating agent-encoding mRNA increased FXN expression approximately 2.5-fold compared to the SH control at 24 hr post-LNP delivery. Modulating agent comprising a fusion molecule comprising dCas9-p300 also slightly upregulated FXN in WT Glutamatergic iNeurons about 1.4-fold using pool1 sgRNAs and the single sgRNA GD-27895 compared to the SH control.

These results demonstrate that modulating agents described herein, in particular a modulating agent comprising dCas9-VPR targeted around 100 bp upstream the TSS, can upregulate FXN gene expression in the cell types (cardiomyocytes and neurons) that are most affected in FRDA.

### Example 3. TAL Effector Molecules Used in Modulating Agents to Increase FXN Expression

This example demonstrates the use of a modulating agent, e.g., fusion molecule, comprising a targeting moiety comprising a TAL effector molecule and an effector moiety comprising VPR to increase FXN expression in FRDA patient-derived fibroblasts.

A modulating agent comprising a fusion molecule comprising a TAL effector molecule fused to a VPR effector moiety was delivered in the form of mRNA to FRDA patient-derived fibroblasts (GM03816). The modulating agent comprising the TAL effector molecule is designed to target about 100 bp upstream of the FXN gene TSS. Modulating agents were formulated in LNPs and then delivered to the cells. In addition, cells treated with modulating agents comprising dCas9-VPR plus pool1 sgRNAs or dCas9-VPR plus safe harbor (SH) sgRNA, as well as untreated cells were used as controls in these experiments.

GM03816 cells were seeded in 96 and 24-well plates in alpha-MEM medium supplemented with 15% FBS. Next day, 112.5 ng and 22.5 ng of LNP (MC3) formulations were added per well to the cells in the 24- and 96-well plates respectively. The medium was changed at 24 hours and samples collected at 24 and 72 hours post-treatment with LNPs.

RNA from samples collected at 24 hr was purified using the RNEasy MiniKit (Qiagen) following the Manufacturer's protocol. RNA samples were retrotranscribed to cDNA using LunaScript RT SuperMix Kit (NEB) and analyzed by quantitative PCR (qPCR) using a FXN-specific Taqman assay with the Taqman Fast Advanced Master Mix (Thermo Scientific). Expression data was analyzed using the standard curve method. FXN expression was normalized to the expression of HPRT1, and fold-change calculated relative to the SH sgRNA-treated control for each timepoint. Fold-change was calculated by dividing normalized FXN mRNA quantity of each sample by the mean of the SH control group.

Protein lysates of samples collected at 72 hr were obtained using RIPA buffer supplemented with protease inhibitors (Roche) and protein concentration quantified using the BCA Rapid Gold Kit (ThermoFisher). Samples were analyzed using the FXN ELISA kit (Abcam ab176112) following the manufacturer's protocol.

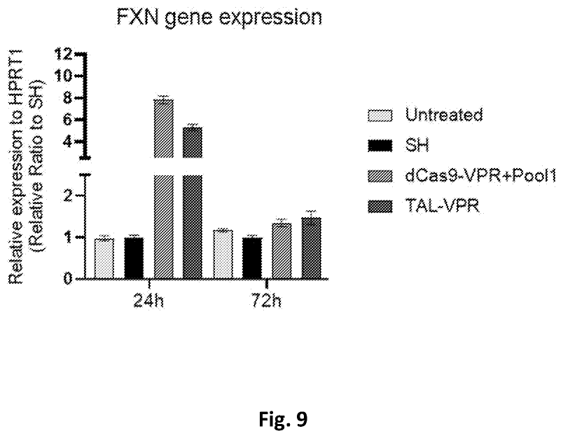

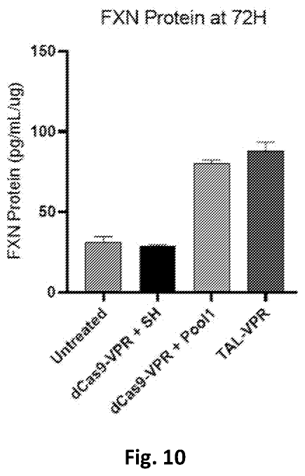

FIGS. 9 and 10 show that modulating agent comprising a fusion molecule comprising TAL-VPR increased FXN gene expression about 6-fold at 24 hr post-LNP delivery and FXN protein about 1.5-fold at 72 hr.

These results showed that modulating agent comprising a fusion molecule comprising TAL-based targeting moieties can deliver effector moieties to region upstream of the TSS and that they can upregulate FXN gene expression.

### Example 4. FXN Expression Changes in Mice Injected with dCas9-VPR

This example demonstrates the use of a modulating agent, e.g., fusion molecule, comprising a targeting moiety comprising dCas9 and an effector moiety comprising VPR to increase FXN expression when injected into mouse model organisms.

3 mg/kg MC3 formulations containing mRNA encoding exemplary modulating agent comprising a fusion molecule comprising dCas9-VPR with GD-28633 sgRNA (targeted ˜80 bp upstream of the Fxn murine TSS) or SH sgRNA were injected intravenously (i.v.) in wild type C57BL/6J mice (N=8 mice/group per timepoint). Additionally, a control group of mice (N=5) injected i.v. with PBS was included for comparison. Liver tissues were collected at day 3, 4, and 5 post-injections and stored in RNA-later for RNA analysis or snap-frozen for protein analysis.

RNA was purified using the RNEasy MiniKit (Qiagen) following the manufacturer's protocol. RNA samples were retrotranscribed to cDNA using LunaScript RT SuperMix Kit (NEB) and analyzed by quantitative PCR (qPCR) using a Fxn specific Taqman assay with Taqman Fast Advanced Master Mix (Thermo Scientific). Expression data was analyzed using the standard curve method. FXN expression was normalized to the expression of HPRT1, and fold-change calculated relative to the SH sgRNA-treated control for each timepoint. Fold-change was calculated by dividing normalized FXN mRNA quantity of each sample by the mean of the SH control group.

Protein lysates were obtained using RIPA buffer supplemented with proteases inhibitors (Roche) and protein concentration quantified using the BCA Rapid Gold Kit (ThermoFisher). Samples were analyzed using the FXN ELISA kit (Abcam ab199078) following the manufacturer's protocol.

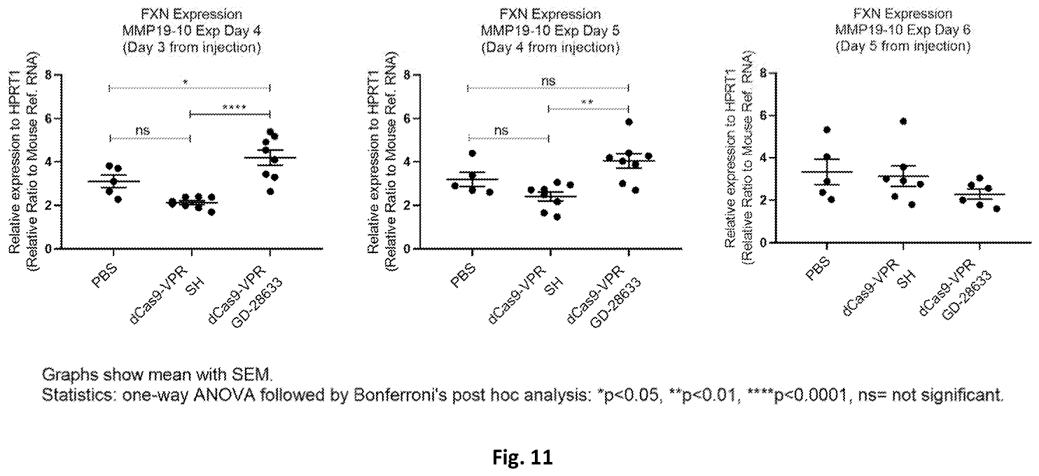

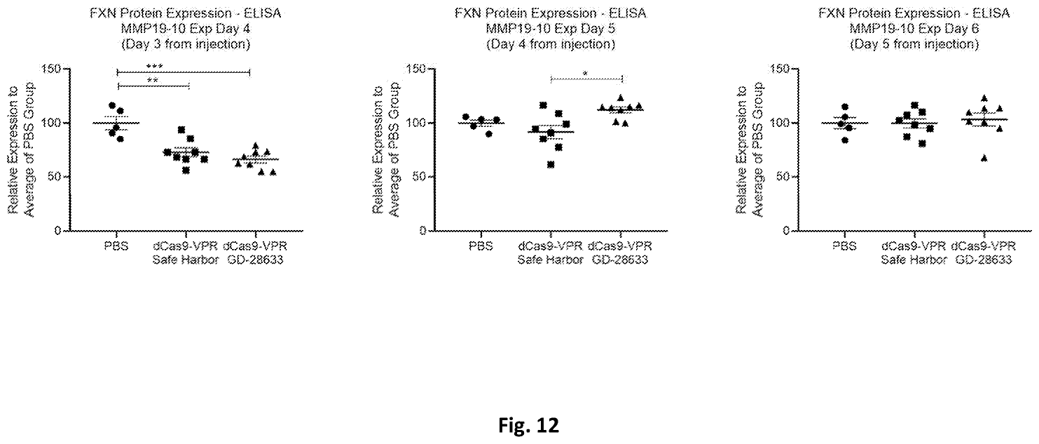

Results in FIG. 11 showed that intravenous injection of LNPs containing dCas9-VPR modulating agent plus GD-28633 sgRNA in mice increased Fxn gene expression approximately 2-fold in liver compared to the dCas9-VPR modulating agent plus SH control at day 3 and 4 after injection. Results in FIG. 12 showed that 4 days post-LNP injection FXN protein levels increased about 20% in the group treated with dCas9-VPR plus GD-28633 as compared to SH control group.

These results demonstrate that a LNP formulation containing a modulating agent comprising a fusion molecule comprising dCas9-VPR plus a sgRNA targeted ˜80 bp upstream of the Fxn murine TSS can increase Fxn gene and protein levels in a surrogate tissue in vivo.

### Example 5. Fxn Expression Changes in Ipscs Derived from Frda Patients

This example demonstrates the use of a modulating agent, e.g., fusion molecule, comprising a targeting moiety comprising dCas9 and an effector moiety comprising either p300 or VPR to increase FXN expression in iPSCs derived from FRDA patients.

Patient (GM04078, GM03816) and wild-type (GM01717, GM03234) iPSC-derived Cardiomyocytes (iCardiomyocytes) were treated with mRNA encoding modulating agent comprising a fusion molecule comprising dCas9-p300 or dCas9-VPR co-delivered with a pool of 3 sgRNAs (pool1). The region targeted by the sgRNAs is about 100 bp upstream of the FXN gene TSS. RNAs were delivered using Lipofectamine MessengerMAX (ThermoFisher) following the manufacturer's protocol. In addition, a safe harbor (SH) sgRNA and untreated cells were used as controls.

Patient (GM04078, GM03816) and wild-type (GM01717, GM03234) iPSC lines were derived from primary fibroblasts using the Stemcell Technologies ReproRNA OKSGM kit following the manufacturer's protocol. iPSC identity was verified with immunocytochemical staining for OCT4, TRA-1-60, SOX2, SSEA4, and NANOG and the parent line was verified using STR.

Patient iPSCs were differentiated into iCardiomyocytes using the STEMdiff Cardiomyocyte Differentiation and Maintenance Kit (Stemcell Technologies). At differentiation day 15, all cells were dissociated and cryopreserved using the STEMdiff Cardiomyocyte Dissociation Kit and STEMdiff Cardiomyocyte Freezing Medium (Stemcell Technologies). Patient/WT iPSC-derived cardiomyocytes were thawed and purified using the StemCell Technologies EasySep Human PSC-Derived Cardiomyocyte Enrichment Kit and plated in Corning Matrigel-coated 24-well plates. Media was changed every other day to fresh Cardiomyocyte Maintenance Media (Stemcell Technologies). 6 days post-plating, when the cells had recovered and started beating, they were treated with 0.5 ug of mRNA in LNP formulations. After 24 hours, the medium was changed and samples collected at 24, 48 and 72 hours post-treatment with LNPs.

RNA was purified from samples using the RNEasy MiniKit (Qiagen) following the manufacturer's protocol. RNA samples were retrotranscribed to cDNA using LunaScript RT SuperMix Kit (NEB) and analyzed by quantitative PCR (qPCR) using a FXN-specific Taqman assay with the Taqman Fast Advanced Master Mix (Thermo Scientific). Expression data was analyzed using the standard curve method. FXN expression was normalized to the expression of HPRT1 or GAPDH, and fold-change calculated relative to the SH sgRNA-treated control for each timepoint. Fold-change was calculated by dividing the normalized FXN mRNA quantity of each sample by the mean of the SH control group.

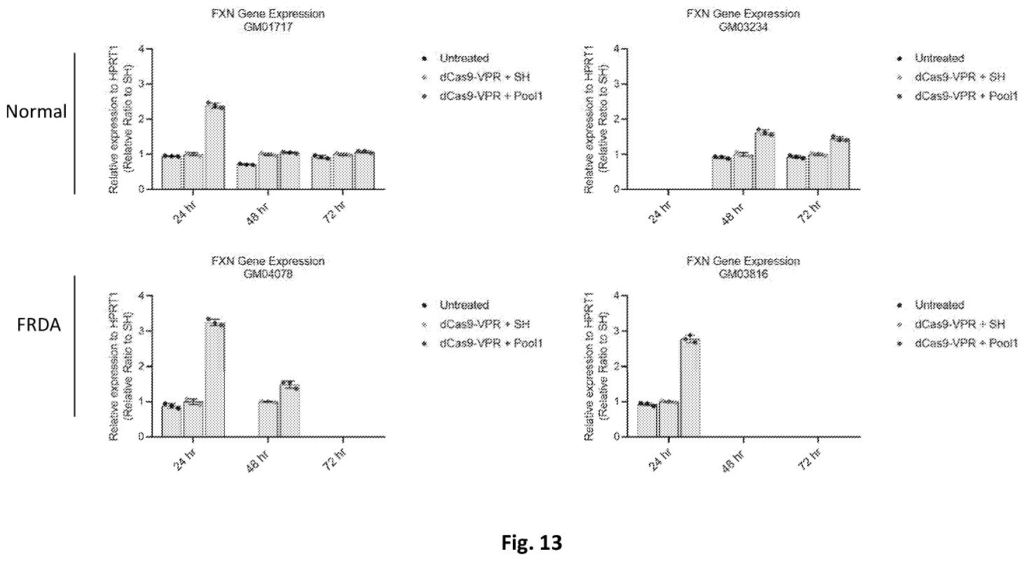

Results in FIG. 13 show a 2.5 to 3.5-fold upregulation of FXN gene expression at 24 hours-post treatment with dCas9-VPR modulating agent-encoding mRNA and Pool1 sgRNA in all cell lines that could be assayed. In control cell line GM01717 the expression returns to baseline by 48 hours. However, approximately 1.5-fold upregulation of FXN is sustained at 48 hours post-treatment in patient cell line GM04078 and at 48 and 72 hours in the wild-type control line GM03234. These data suggest the modulating agent comprising a fusion molecule comprising dCas9-VPR and pool1 sgRNA induced upregulation of FXN that may be more durable in some lines.

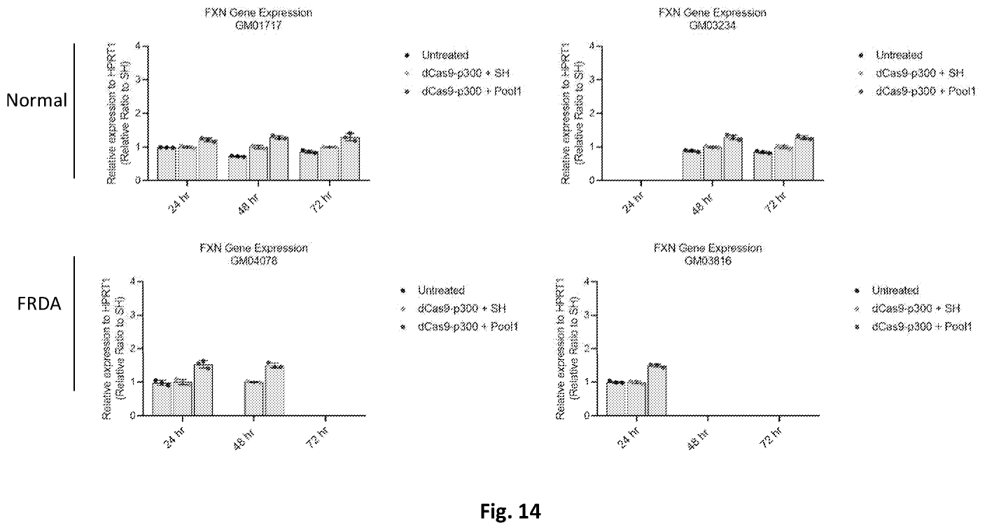

Results in FIG. 14 show an approximately 1.5-fold upregulation of FXN gene expression after treatment with dCas9-p300 modulating agent-encoding mRNA and pool1 sgRNA in both patient cell lines, GM04078 and GM03816. FXN expression was upregulated by about 1.3-fold in the two wild-type lines, GM01717 and GM03234. Based on the available data, the modulating agent comprising a fusion molecule comprising p300 mediated upregulation of FXN that appears to remain stable at 72 hours, suggesting it may have a more durable effect on FXN gene expression in cardiomyocytes.

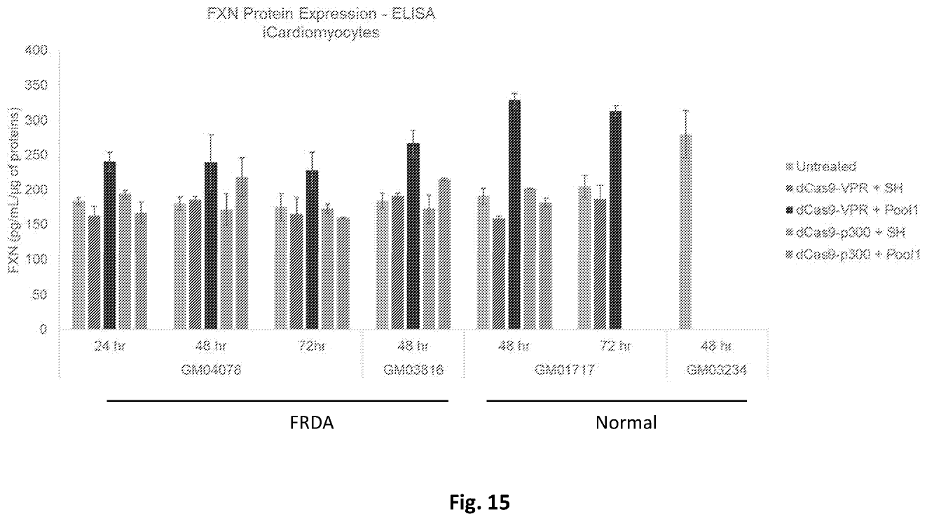

Results in FIG. 15 show that delivery of sgRNA pool1 together with dCas9-VPR modulating agent-encoding mRNA increases FXN protein levels in patient and wild-type iCardiomyocytes up to 72 hours. sgRNA pool1 together with dCas9-p300 modulating agent-encoding mRNA also slightly upregulates FXN protein in the patient cell lines.

These results demonstrate that modulating agents described herein (e.g., comprising a fusion molecule), in particular comprising dCas9-VPR and targeted approximately 100 bp upstream of the TSS, can upregulate FXN gene and protein expression in one of the cell types (cardiomyocytes) that are most affected in FRDA.

We claim:

1. A modulating agent comprising:
a targeting moiety that binds to an expression control element of the frataxin (FXN) gene, and
an effector moiety comprising an epigenetic modifying moiety capable of increasing expression of FXN.

2. A modulating agent comprising:
a targeting moiety that binds to an expression control element of the frataxin (FXN) gene,
a first effector moiety capable of increasing expression of FXN, and
a second effector moiety capable of increasing expression of FXN,
wherein the first and second effector moieties are different moieties.

3. A modulating agent comprising:
a targeting moiety that binds to an expression control element of the frataxin (FXN) gene, wherein the targeting moiety comprises a Zn Finger molecule, and
an effector moiety capable of increasing expression of FXN.

4. A nucleic acid encoding a modulating agent of any of claims 1-3.

5. A recombinant RNA encoding a modulating agent, wherein the modulating agent comprises:
a targeting moiety that binds to an expression control element of the frataxin (FXN) gene, and
an effector moiety capable of increasing expression of FXN.

6. A nanoparticle (e.g., a lipid nanoparticle (LNP)) comprising a nucleic acid, e.g., a recombinant RNA, encoding a modulating agent, the modulating agent comprising:
a targeting moiety that binds to an expression control element of the frataxin (FXN) gene, and
an effector moiety capable of increasing expression of FXN.

7. A method of increasing frataxin (FXN) expression in a cell, comprising:
contacting a cell with a modulating agent, the modulating agent comprising:
a targeting moiety that binds to an expression control element of the frataxin (FXN) gene, and
an effector moiety capable of increasing expression of FXN,
thereby increasing FXN expression in the cell,
wherein FXN expression increases for at least 1, 2, 3, 4, 5, 6, 7, 8, 9, or 10 weeks (and optionally, permanently), or
wherein the cell comprises a FXN allele comprising a GAA expansion of at least 44 copies, wherein after treatment with the modulating agent the FXN allele is expressed at a level of at least 1.5× (i.e., 1.5 times) the expression level of a similar cell not contacted with the modulating agent.

8. A method of increasing frataxin (FXN) expression in a cell, comprising:
contacting a cell with the modulating agent, nucleic acid, recombinant RNA, nanoparticle, or viral vector of any of claims 1-7,
thereby increasing FXN expression in the cell.

9. The modulating agent, nucleic acid, recombinant RNA, nanoparticle, viral vector, or method of any of claims 1-8, wherein the effector moiety comprises an epigenetic modifying moiety.

10. The modulating agent, nucleic acid, recombinant RNA, nanoparticle, viral vector, or method of any of claims 1-9, wherein the epigenetic modifying moiety comprises a DNA methyltransferase, a histone methyltransferase, a DNA demethylase, a histone acetyltransferase, or a functional fragment or variant of any thereof.

11. The modulating agent, nucleic acid, recombinant RNA, nanoparticle, viral vector, or method of any of claims 1-10, wherein the effector moiety comprises a DNA demethylase or functional fragment or variant thereof, e.g., a protein chosen from TET1, TET2, TET3, or TDG, or a functional variant or fragment of any thereof.

12. The modulating agent, nucleic acid, recombinant RNA, nanoparticle, viral vector, or method of any of claims 1-11, wherein the effector moiety comprises a histone methyltransferase or functional fragment or variant thereof, e.g., a protein chosen from DOT1L, PRDM9, PRMT1, PRMT2, PRMT3, PRMT4, PRMT5, NSD1, NSD2, NSD3, or a functional variant or fragment of any thereof.

13. The modulating agent, nucleic acid, recombinant RNA, nanoparticle, viral vector, or method of any of claims 1-12, wherein the effector moiety comprises a histone acetyltransferase or functional fragment or variant thereof, e.g., a protein chosen from p300, CREB-binding protein (CBP), or functional fragment or variant thereof.

14. The modulating agent, nucleic acid, recombinant RNA, nanoparticle, viral vector, or method of any of claims 1-13, wherein the effector moiety comprises a transcriptional activator or functional fragment or variant thereof, e.g., a protein chosen from VP16, VP64, VP160, or VPR.

15. The modulating agent, nucleic acid, recombinant RNA, nanoparticle, viral vector, or method of any of claims 1-14, wherein the targeting moiety comprises a Cas9 molecule.

16. The modulating agent, nucleic acid, recombinant RNA, nanoparticle, viral vector, or method of claim 15, wherein the Cas9 molecule comprises a Cas9 protein from Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

17. The modulating agent, nucleic acid, recombinant RNA, nanoparticle, viral vector, or method of either claim 15 or 16, wherein the Cas9 molecule comprises a Cas9 protein substantially lacking nuclease activity, e.g., dCas9, e.g., comprising inactive RuvC and/or HNH domains.

18. The modulating agent, nucleic acid, recombinant RNA, nanoparticle, viral vector, or method of any of claims 15-17 wherein the Cas9 molecule comprises (e.g., is noncovalently bound to) a gRNA, e.g., an sgRNA, wherein the gRNA binds to the expression control element.

19. The modulating agent, nucleic acid, recombinant RNA, nanoparticle, viral vector, or method of claim 18, wherein the gRNA comprises a nucleic acid sequence selected from any of SEQ ID NOs: 4-26, or a sequence with at least 80, 85, 90, 95, or 99% identity to any of SEQ ID NOs: 4-26.

20. The modulating agent, nucleic acid, recombinant RNA, nanoparticle, viral vector, or method of any of claims 1-14, wherein the targeting moiety comprises a TAL effector molecule.

21. The modulating agent, nucleic acid, recombinant RNA, nanoparticle, viral vector, or method of any of claims 1-14, wherein the targeting moiety comprises a Zn Finger molecule.

22. The modulating agent, nucleic acid, recombinant RNA, nanoparticle, viral vector, or method of any of claims 1-14, wherein the targeting moiety comprises a Zn Finger molecule that comprises 2, 3, 4, 5, or 6 Zn finger proteins.

23. The modulating agent, nucleic acid, recombinant RNA, nanoparticle, viral vector, or method of any of claims 1-22, wherein the expression control element comprises an enhancer or promoter or portion thereof operably linked to the FXN gene.

24. The modulating agent, nucleic acid, recombinant RNA, nanoparticle, viral vector, or method of any of claims 1-23, wherein the targeting moiety binds to a nucleic acid sequence that is no more than 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10, 9, 8, 7, 6, 5, 4, 3, 2, or 1 nucleotides upstream or downstream from the transcription start site of the FXN gene (and optionally at least 1, 2, 3, 4, 5, 6, 7, 8, 9, 10, 20, 30, 40, 50, 60, 70, 80, or 90 nucleotides upstream or downstream).

25. The modulating agent, nucleic acid, recombinant RNA, nanoparticle, viral vector, or method of any of claims 1-24, wherein the targeting moiety binds to a nucleic acid sequence selected from a sequence denoted by genomic coordinates of Table 3.

26. The modulating agent, nucleic acid, recombinant RNA, nanoparticle, viral vector, or method of any of claims 1-25,
wherein the targeting moiety comprises a Cas9 molecule, e.g., a dCas9 molecule, and the effector moiety comprises p300 or a functional fragment or variant thereof.

27. The modulating agent, nucleic acid, recombinant RNA, nanoparticle, viral vector, or method of any of claims 1-25,
wherein the targeting moiety comprises a Cas9 molecule, e.g., a dCas9 molecule, and the effector moiety comprises VP64 or a functional fragment or variant thereof.

28. The modulating agent, nucleic acid, recombinant RNA, nanoparticle, viral vector, or method of any of claims 1-25,
wherein the targeting moiety comprises an enzymatically inactive Cas nuclease, e.g., a dCas9 molecule, and the effector moiety comprises VP64 or a functional fragment or variant thereof, p65 or a functional fragment or variant thereof, and RTA or a functional fragment or variant thereof.

29. The modulating agent, nucleic acid, recombinant RNA, nanoparticle, viral vector, or method of any of claims 1-25,
wherein the targeting moiety comprises a TAL effector molecule (e.g., wherein the TAL effector molecule binds upstream of the FXN gene TSS, e.g., about 50-150 nucleotides upstream, e.g., about 100 nucleotides upstream), and the effector moiety comprises VPR or a functional fragment or variant thereof.

30. The modulating agent, nucleic acid, recombinant RNA, nanoparticle, viral vector, or method of any of claims 1-25,
wherein the targeting moiety comprises a TAL effector molecule molecule (e.g., wherein the TAL effector molecule binds upstream of the FXN gene TSS, e.g., about 50-150 nucleotides upstream, e.g., about 100 nucleotides upstream), and the effector moiety comprises VP64 or a functional fragment or variant thereof, p65 or a functional fragment or variant thereof, and RTA or a functional fragment or variant thereof.

31. The modulating agent, nucleic acid, recombinant RNA, nanoparticle, viral vector, or method of any of claims 1-25,
wherein the targeting moiety comprises a Zn finger molecule (e.g., wherein the Zn finger molecule binds upstream of the FXN gene TSS, e.g., about 50-150 nucleotides upstream, e.g., about 100 nucleotides upstream), and the effector moiety comprises VPR or a functional fragment or variant thereof.

32. The modulating agent, nucleic acid, recombinant RNA, nanoparticle, viral vector, or method of any of claims 1-25,
wherein the targeting moiety comprises a Zn finger molecule molecule (e.g., wherein the Zn finger molecule binds upstream of the FXN gene TSS, e.g., about 50-150 nucleotides upstream, e.g., about 100 nucleotides upstream), and the effector moiety comprises VP64 or a functional fragment or variant thereof, p65 or a functional fragment or variant thereof, and RTA or a functional fragment or variant thereof.

33. The modulating agent, nucleic acid, recombinant RNA, nanoparticle, viral vector, or method of any of claims 1-32, wherein the modulating agent comprises or is a fusion molecule.

34. The modulating agent, nucleic acid, recombinant RNA, nanoparticle, viral vector, or method of any of claim 1-25 or 33, wherein the modulating agent comprises an amino acid sequence selected from any of SEQ ID NOs: 304-309, or an amino acid sequence with at least 80, 85, 90, 95, 96, 97, 98, or 99% identity thereto.

35. A complex comprising a modulating agent of any of claim 1-3 or 9-34 and a nucleic acid sequence comprising the expression control sequence of the FXN gene.

36. A cell comprising the modulating agent, nucleic acid, recombinant RNA, nanoparticle, or viral vector of any of claim 1-6 or 9-34.

37. A cell comprising a nucleic acid encoding the modulating agent of any of claim 1-3 or 9-34.

38. A method of delivering a modulating agent, nucleic acid, recombinant RNA, nanoparticle, or viral vector of any of claim 1-6 or 9-34 to a cell, comprising:
contacting the cell with the modulating agent, nucleic acid, recombinant RNA, nanoparticle, or viral vector,
thereby delivering the modulating agent, nucleic acid, recombinant RNA, nanoparticle, or viral vector to the cell.

39. A method of modulating, e.g., increasing, transcription of the frataxin (FXN) gene, comprising:
contacting a cell with the modulating agent, nucleic acid, recombinant RNA, nanoparticle, or viral vector of any of claim 1-6 or 9-34,
thereby modulating, e.g., increasing, expression of the FXN gene.

40. A method of treating a patient having Friedrich's Ataxia (FRDA), comprising:
administering a modulating agent, nucleic acid, recombinant RNA, nanoparticle, or viral vector of any of claim 1-6 or 9-34 to the patient,
thereby treating the patient.

41. The method of any of claims 38-40, wherein the method increases FXN levels in blood (e.g., whole blood) by at least 10, 20, 30, 40, 50, 60, 70, 80, 90, 100, 120, 140, 160, 180, 200, 300, or 400% relative to FXN levels in blood (e.g., whole blood) in the absence of the modulating agent, nucleic acid, recombinant RNA, nanoparticle, or viral vector.

42. The method of any of claims 38-41, wherein the method lessens or eliminates at least one symptom of FDRA, e.g., a symptom selected from ataxia, dysarthria, muscle weakness, spasticity (e.g., lower limb spasticity), scoliosis, bladder dysfunction, reflex dysfunction, loss of position and/or vibration sense, cardiomyopathy, or diabetes mellitus.

43. The method of either of claim 41 or 42, wherein the level of FXN in blood (e.g., whole blood) is increased for at least 1, 2, 3, 4, 5, 6, 7, 8, 9, or 10 weeks, or at least 1, 2, 3, 4, 5, 6, 7, 8, 9, 10, 11, or 12 months, or at least 1, 2, 3, 4, or 5 years.

44. The method of any of claims 38-43, wherein the method increases FXN levels in blood (e.g., whole blood) for at least 12, 18, 24, 30, 36, 42, 48, 54, 60, 66, 72, 78, 84, 90, or 96 hours.
